(19) United States
(12) Patent Application Publication
(10) Pub. No.: US 2025/0304570 A1
(43) Pub. Date: Oct. 2, 2025

(54) BENZO-FUSED N-HETEROCYCLES AND USES THEREOF

(71) Applicant: Kumquat Biosciences Inc., San Diego, CA (US)

(72) Inventors: Pingda Ren, San Diego, CA (US); Liansheng Li, San Diego, CA (US)

(21) Appl. No.: 19/075,486

(22) Filed: Mar. 10, 2025

Related U.S. Application Data
(63) Continuation of application No. PCT/US2023/032491, filed on Sep. 12, 2023.
(60) Provisional application No. 63/406,216, filed on Sep. 13, 2022.

(51) Int. Cl. C07D 417/04; A61K 31/498; A61K 31/502; A61K 31/517; A61K 31/5377; A61P 35/00; C07D 417/14

(52) U.S. Cl. CPC C07D 417/04; A61K 31/498; A61K 31/502; A61K 31/517; A61K 31/5377; A61P 35/00; C07D 417/14

(57) ABSTRACT

The present disclosure provides compounds and pharmaceutical compositions comprising the same. The compounds, pharmaceutical compositions thereof, and methods of using the same have a range of utilities as therapeutics, diagnostics, and research tools. The subject compositions and methods are particularly useful for potentiating immune response and/or for treating cancer and other diseases.

### CROSS-REFERENCE

This application is a continuation of International Application No. PCT/US2023/032491, filed Sep. 12, 2023, which claims the benefit of U.S. Provisional Application No. 63/406,216, filed Sep. 13, 2022, each of which is incorporated herein by reference in its entirety.

### BACKGROUND

PTPN2 encodes a protein tyrosine phosphatase that has been implicated in a number of intracellular signaling pathways of immune cells. PTPN2 can negatively regulate αβ TCR T cell receptor (TCR) signaling by dephosphorylating and inactivating, e.g., the Src family kinase including LCK. In addition, PTPN2 can antagonize growth factor or cytokine-mediated signaling required for T cell function, homeostasis, and/or differentiation by dephosphorylating and inactivating JAK family kinases, e.g., JAK-1 and JAK-3, and/or target substrates of the JAK family kinases, e.g., STAT-1, STAT-3, and STAT-5.

Based on genome-wide association studies, PTPN2 single nucleotide polymorphisms (SNPs) have been linked with the development of several human autoimmune diseases including, but not limited to, type 1 diabetes, rheumatoid arthritis, Crohn's disease, and celiac disease. For example, a PTPN2 variant, rs1893217 (C), has been associated with about a 40% decrease in PTPN2 mRNA expression in CD4+ T cells, as well as the development of type 1 diabetes. In addition, PTPN2 mRNA expression levels in lung cancer tissues have been shown to be higher than those in normal lung tissues or adjacent normal tissues, such overexpression of PTPN2 promoting proliferation of lung cancer cells. Furthermore, two PTPN2 SNPs, rs2847297 and rs2847282, have been associated with a decrease in both PTPN2 mRNA expression and lung cancer risk, especially squamous cell lung carcinoma risk.

Cancer is the second leading cause of human death. There were close to 10 million deaths from cancer worldwide in 2018 and 17 million new cases were diagnosed. In the United States alone, cancer causes the death of over a half-million people annually, with some 1.7 million new cases diagnosed per year (excluding basal cell and squamous cell skin cancers). Lung, liver, stomach, and bowel cancers account for more than four in ten of all cancer deaths worldwide.

Adoptive transfer of gene modified lymphoid cells, particularly T cells (i.e., ACT), is an emerging treatment for cancer. While efficacy has been demonstrated in a range of hematological cancers including ALL, CLL, DLBCL, FL, and multiple myeloma, its efficacy in treating solid tumors is still yet to be established. Current immune cell therapy (e.g., CAR-T therapy) suffers from a number of profound deficiencies. T cell manufacturing and clonal expansion are highly inefficient and costly. When introduced in to a patient, T cell's anti-tumor activity and numbers can be reduced in the immunosuppressive microenvironment often found in a tumor. In addition, CAR-T therapy has been limited by life threatening toxicities in over 30% of patients. Toxicities primarily manifest as cytokine release syndrome (CRS) characterized by an early phase with fever, hypotension and elevations of various cytokines, and a later phase associated with life-ending neurologic events.

### SUMMARY

In view of the foregoing, there exists a considerable need for alternative compositions and methods to treat cancer, and/or carry out immunotherapy. The compositions and methods of the present disclosure address this need and provide additional advantages as well. The ability of PTPN2 to act as a negative regulator of immunoreceptor-related pathways (e.g., TCR signaling) and promote cancer cell proliferation can be exploited for cancer and tumor treatment. The various aspects of the disclosure provide compositions and methods for inducing activity of lymphoid cells.

In certain aspects, the present disclosure provides a compound of Formula (I):

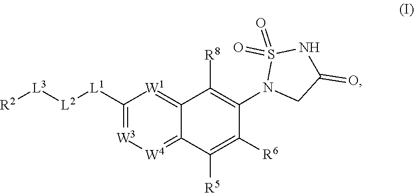

or a pharmaceutically acceptable salt or solvate thereof, wherein:
W¹ is N, W³ is N, and W⁴ is C(R⁴); W¹ is N, W³ is C(R³), and W⁴ is N; or W¹ is C(R¹), W³ is N, and W⁴ is N;
R¹, R³, R⁴, R⁵, R⁶, and R⁸ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
L¹ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—;
L² is selected from C₁₋₆ alkylene, C₂₋₆ alkenylene, C₂₋₆ alkynylene, —C₀₋₃ alkylene-C₃₋₈ carbocycle-, and —C₀₋₃ alkylene-(3- to 8-membered heterocycle)-, each of which is optionally substituted with one, two, or three R²⁰;
L³ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—;
R² is selected from hydrogen, halogen, —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R₁₃ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, —C₀₋₃ alkyl-C₃₋₁₀ carbocycle, —C₀₋₃ alkyl-(3- to 10-membered heterocycle), —OR²², —SR²², —N(R²²)(R²³), —NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², —C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, C₃₋₁₀ carbocycle, and 3- to 10-membered heterocycle, or two R²¹ are taken together with the carbon atom to which they are attached to form C₃₋₈ carbocycle or 3- to 8-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and

R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

For a compound of Formula (I), (₁a), (I-1), (I-1a), (I-2), (I-2a), R¹, R³, and R⁴ may independently be selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6-membered heterocycle are optionally substituted with one, two, or three R²⁰. In some embodiments, R¹, R³, and R⁴ are independently selected from hydrogen, halogen, C₁₋₃ alkyl, C₁₋₃ haloalkyl, C₃₋₆ carbocycle, —OH, —OCH₃, —NH2, and —NHCH3. In some embodiments, R¹ is selected from hydrogen, chlorine, and fluorine, such as R¹ is hydrogen. In some embodiments, R³ is selected from hydrogen, —OH, and —NH2, such as R³ is hydrogen. In some embodiments, R⁴ is hydrogen.

The compound of Formula (I) may be a compound of Formula (I-A):

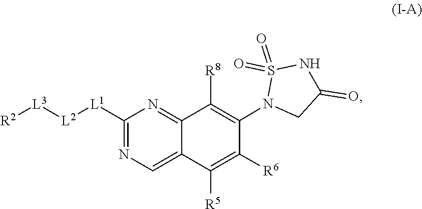

or a pharmaceutically acceptable salt or solvate thereof.

The compound of Formula (I) may be a compound of Formula (I-B):

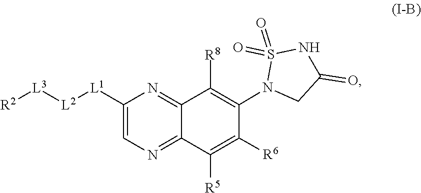

or a pharmaceutically acceptable salt or solvate thereof.

The compound of Formula (I) may be a compound of Formula (I-C):

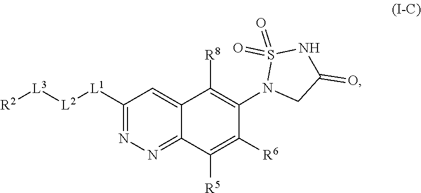

or a pharmaceutically acceptable salt or solvate thereof.

For a compound of Formula (I), (I-1), (I-2), (I-A), (I-A1), (I-A2), (I-B), (I-B1), (I-B2), (I-C), (I-C1), or (I-C2), R⁵ may be selected from hydrogen, halogen, C₁₋₆ alkyl, C₃₋₆ carbocycle, 3- to 6-membered heterocycle, —OR¹², and —N(R¹²)(R¹³), wherein C₁₋₆ alkyl, C₃₋₆ carbocycle, and 3- to 6-membered heterocycle are optionally substituted with one, two, or three R²⁰. In some embodiments, R⁵ is selected from hydrogen, halogen, and —OH, such as R⁵ is hydrogen. In some embodiments, R⁶ is selected from halogen, —OR¹², and C₁₋₆ alkyl, wherein C₁₋₆ alkyl is optionally substituted with one, two, or three R²⁰; such as R⁶ is —OH. In some embodiments, R⁸ is selected from halogen, —OR¹², and C₁₋₆ alkyl, wherein C₁₋₆ alkyl is optionally substituted with one, two, or three R²⁰. In some embodiments, R⁸ is halogen, such as R⁸ is fluorine. In some embodiments, R⁵ is hydrogen, R⁶ is —OH, and R⁸ is fluorine. In some embodiments, R⁵ is —OH, R⁶ is hydrogen, and R⁸ is fluorine.

For a compound of Formula (I), (Ia), (I-A), (I-Aa), (I-B), (I-Ba), (I-C), or (I-Ca), L¹ may be selected from absent, —O—, —S—, —N(R¹²)—, —C(NR¹²)—, —N(R¹²)S(O)₂—, —S(O)—, —S(O)₂—, and —S(O)₂N(R¹²)—. In some embodiments, L¹ is selected from absent, —O—, and —N(R¹²)—. In some embodiments, L¹ is selected from —O— and —N(R¹²)—, such as L¹ is absent. In some embodiments, L¹ is —O—. In some embodiments, L¹ is —N(R¹²)—.

For a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L² may be selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰. In some embodiments, the C₃₋₈ carbocycle of L² is selected from C₃₋₈ monocyclic cycloalkyl, C₅₋₈ monocyclic cycloalkenyl, and C₆ monocyclic aryl, each of which is optionally substituted with one, two, or three R²⁰; and the 3- to 8-membered heterocycle of L² is selected from 3- to 8-membered monocyclic heterocycloalkyl, 5- to 8-membered monocyclic heterocycloalkenyl, and 5- to 6-membered monocyclic heteroaryl, each of which is optionally substituted with one, two, or three R²⁰. In some embodiments, L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵.

For a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L³ may be selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—. In some embodiments, L³ is selected from absent, —N(R¹²)—, —C(O)O—, —OC(O)—, and —S(O)₂—, such as L³ is absent.

In some embodiments, for a compound of Formula (I), (Ia), (I-A), (I-Aa), (I-B), (I-Ba), (I-C), or (I-Ca), L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L¹ is selected from —O—, and —N(R¹²)—; L² is selected from C₁₋₆ alkylene, —C₀₋₃ alkylene-C₃-& carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L¹ is absent; L² is 3- to 8-membered heterocycle, optionally substituted with one, two, or three R²⁰; and L³ is selected from absent and —S(O)₂—.

For a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), R² may be selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, R² is selected from hydrogen, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₆ alkyl, —C₀₋₃ alkyl-C₃₋₆ carbocycle, —C₀₋₃ alkyl-(3- to 6-membered heterocycle), —OR²², —N(R²²)(R²³), ═NR²², ═C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—, wherein C₁₋₆ alkyl, —C₀₋₃ alkyl-C₃₋₆ carbocycle, and —C₀₋₃ alkyl-(3- to 6-membered heterocycle) are optionally substituted with one, two, or three substituents independently selected from halogen, oxo, —CN, C₁₋₆ alkyl, —OR²², —N(R²²)(R²³), ═NR²², —C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—. In some embodiments, R² is selected from hydrogen, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH2, such as R² is hydrogen.

In some embodiments, for a compound of Formula (I), (Ia), (I-A), (I-Aa), (I-B), (I-Ba), (I-C), or (I-Ca), —L¹-L²-L³-R² is selected from

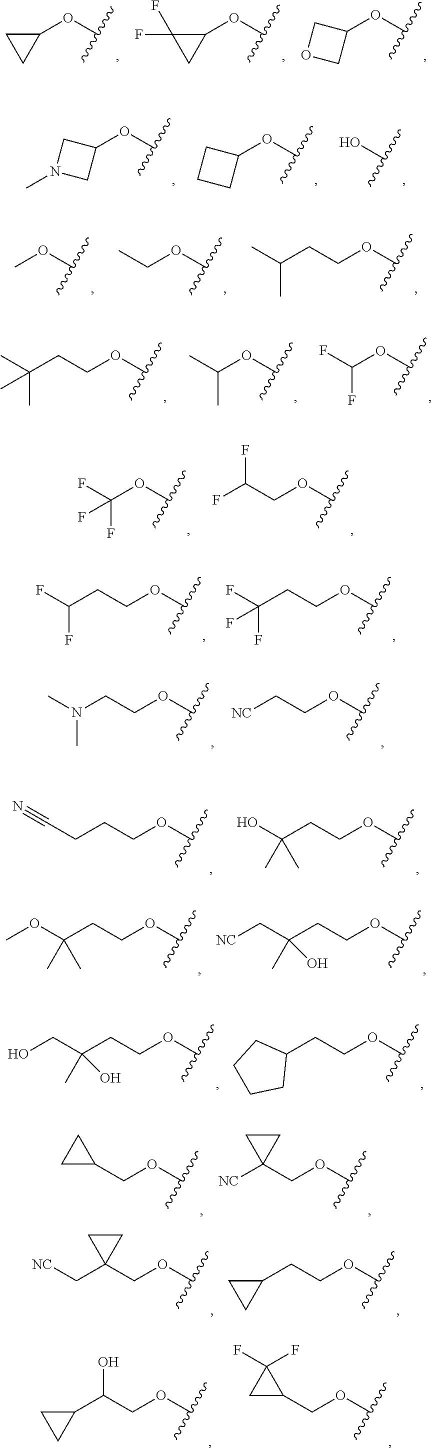

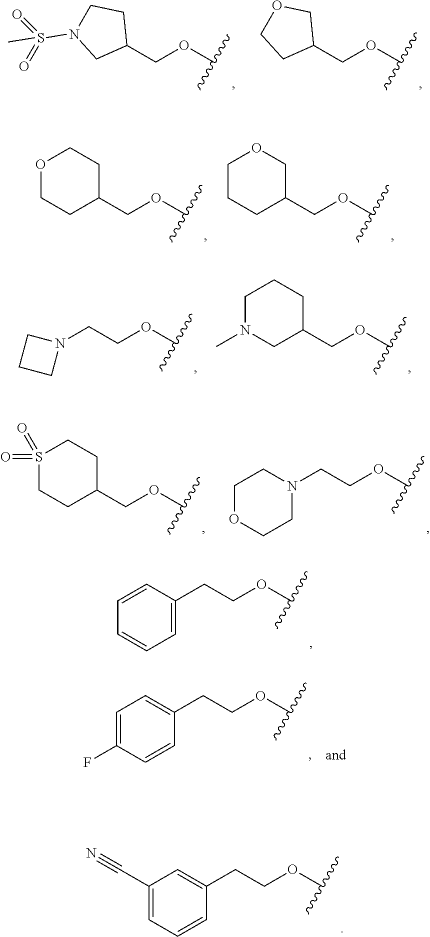

In some embodiments, for a compound of Formula (I), (Ia), (I-A), (I-Aa), (I-B), (I-Ba), (I-C), or (I-Ca), —L¹-L²-L³-R² is selected from

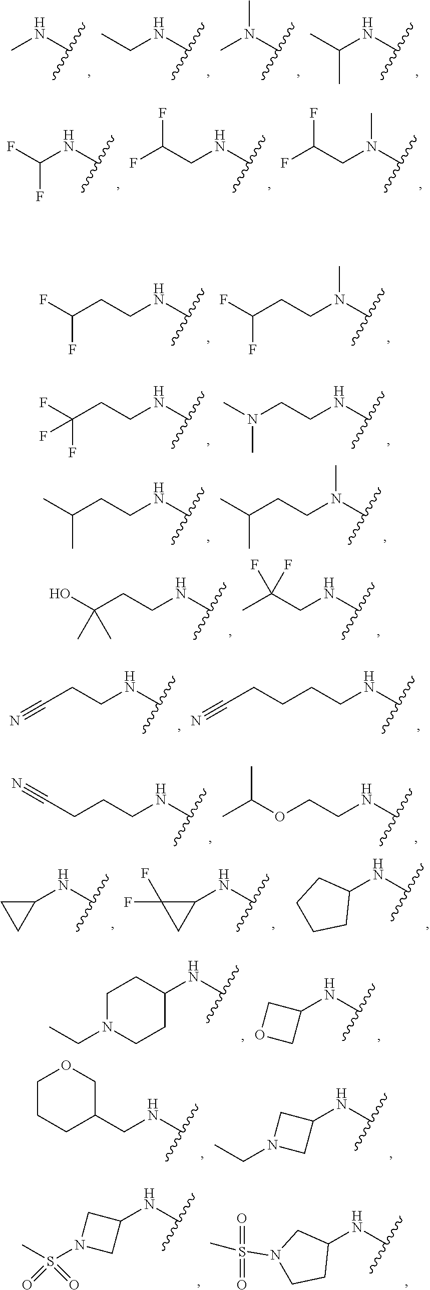

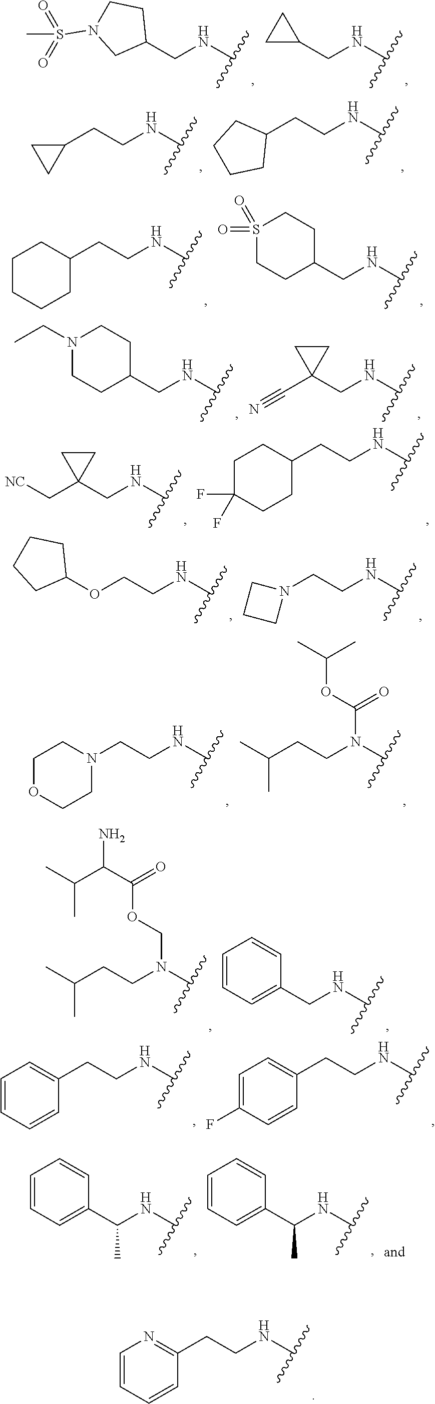

In some embodiments, for a compound of Formula (I), (Ia), (I-A), (I-Aa), (I-B), (I-Ba), (I-C), or (I-Ca), —L¹-L²-L³-R² is selected from

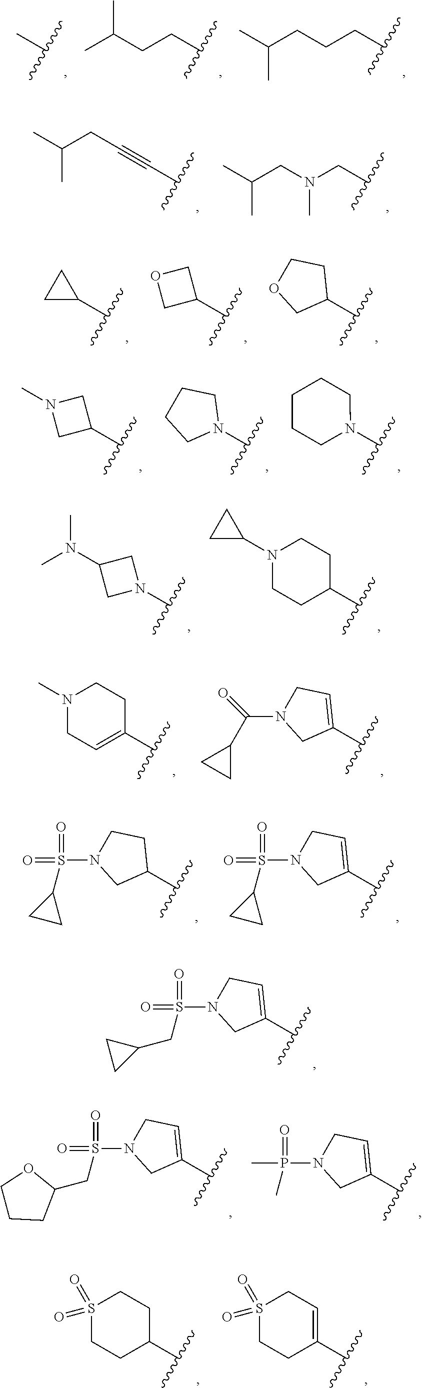

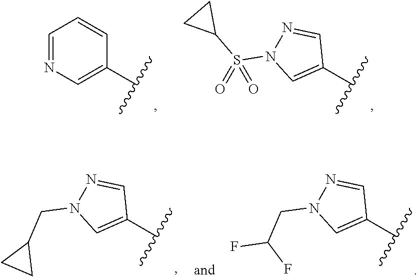 [SMILES: *c1cccnc1 ; *c1cnn(S(=O)(=O)C2CC2)c1 ; *c1cnn(CC2CC2)c1 ; *c1cnn(CC(F)F)c1]

In some embodiments, for a compound of Formula (I), (Ia), (I-A), (I-Aa), (I-B), (I-Ba), (I-C), or (I-Ca), —L¹-L²-L³-R² is selected from

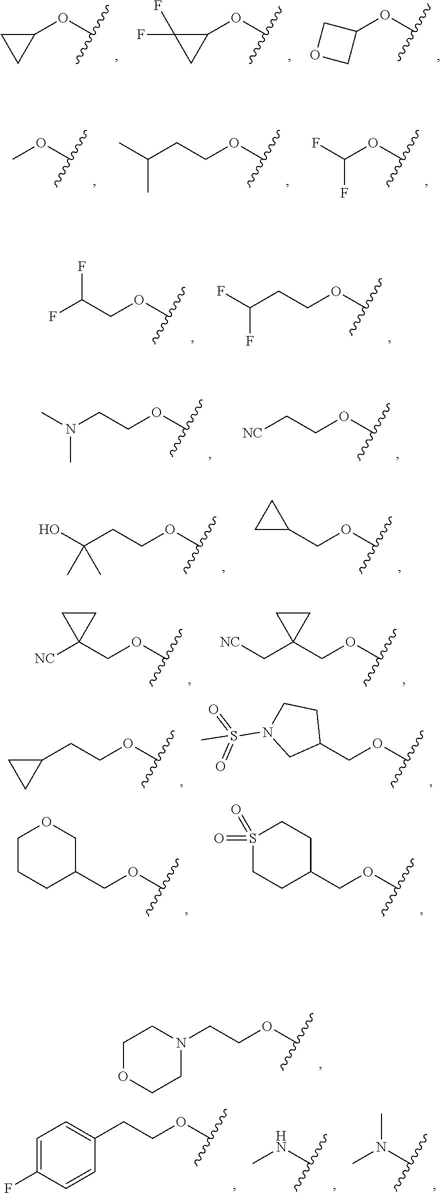

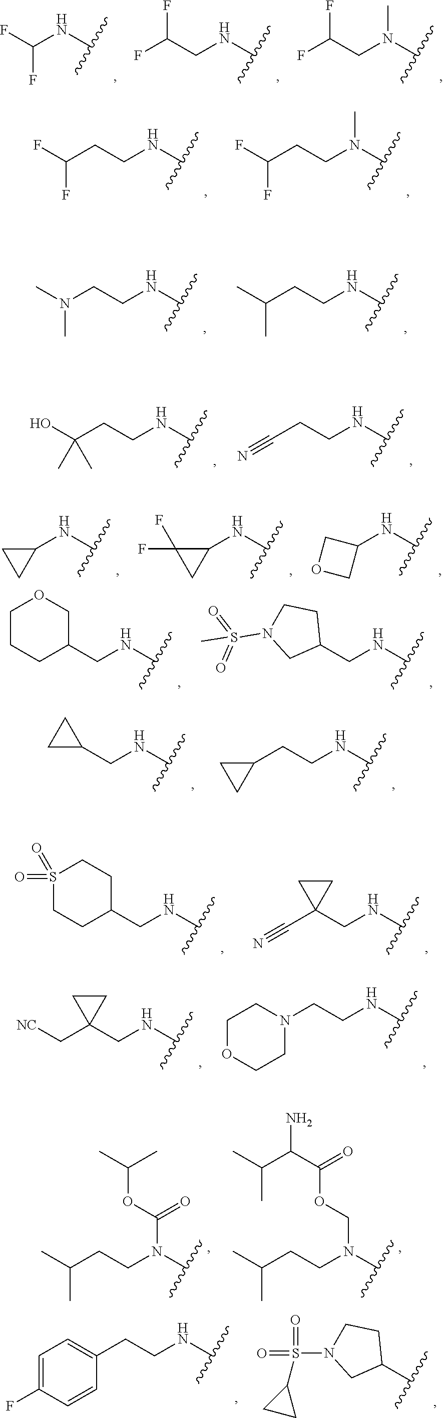

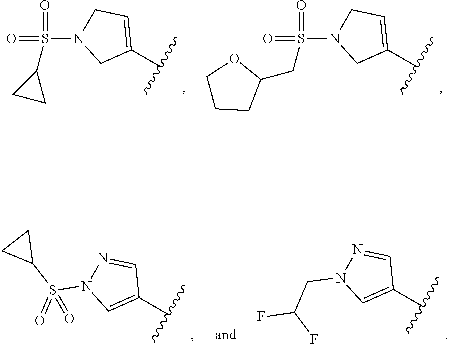 [SMILES: *C1=CCN(S(=O)(=O)C2CC2)C1 ; *C1=CCN(S(=O)(=O)CC2CCCO2)C1 ; *c1cnn(S(=O)(=O)C2CC2)c1 ; *c1cnn(CC(F)F)c1]

In certain aspects, the present disclosure provides a compound described herein, or a pharmaceutically acceptable salt or solvate thereof. In certain aspects, the present disclosure provides a pharmaceutical composition comprising a compound described herein, or a pharmaceutically acceptable salt or solvate thereof, and a pharmaceutically acceptable excipient.

In certain aspects, the present disclosure provides a method of treating cancer in a subject in need thereof, comprising administering to the subject a therapeutically effective amount of a compound described herein, or a pharmaceutically acceptable salt or solvate thereof. In certain aspects, the present disclosure provides a method of potentiating immunity of a cell, comprising (a) contacting the cell with a compound described herein, thereby potentiating immunity of the cell, wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In certain aspects, the present disclosure provides a method of potentiating immunity of a cell, comprising: (a) contacting the cell with a compound described herein; and (b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell. In some embodiments, (a) is performed prior to, concurrent with, or subsequent to (b). In some embodiments, the cell retains expression or activity of PTPN2 prior to (a). In some embodiments, the cell is a lymphoid cell. In some embodiments, the method further comprises administering the cell to a subject in need thereof. In some embodiments, the method further comprises administering a compound described herein to the subject prior to, concurrent with, or subsequent to the administering the cell. In some embodiments, prior to the administering the compound described herein, a cell of the subject exhibits expression or activity of PTPN2.

In certain aspects, the present disclosure provides a method of treating cancer in a subject in need thereof, comprising: (a) administering systemically a compound described herein; and (b) administering a second agent or a second therapy concurrently, before, or after step (a), wherein the second agent or the second therapy comprises a lymphoid cell that (1) retains expression or activity of PTPN2 prior to being exposed to the compound, and (2) expresses (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to a tumor antigen. In some embodiments, the compound is administered systemically and transiently to the subject in need thereof, and wherein the second agent or the second therapy comprises a lymphoid cell that (1) retains expression or activity of PTPN2 prior to being exposed to the compound, and (2) a chimeric antigen receptor (CAR) sequence encoding a CAR that exhibits specific binding to a tumor antigen. In some embodiments, prior to being exposed to the compound, the lymphoid cell retains at least about 90% of the expression or activity of PTPN2 as compared to a control. In some embodiments, the second agent or the second therapy comprises a sub-therapeutic amount of the lymphoid cells. In some embodiments, the compound (i) does not regulate site-specific recombination of a gene encoding PTPN2, and (ii) does not affect editing of the gene encoding PTPN2. In some embodiments, the lymphoid cell is an immune effector cell. In some embodiments, the lymphoid cell is selected from the group consisting of: T cell, B cell, NK cell, KHYG cell, T helper cell, regulatory T cell, memory T cell, tumor infiltration T cell (TIL), antigen presenting cell, and dendritic cell. In some embodiments, the lymphoid cell is selected from the group consisting of a CD4+ T cell, a CD8+ T cell, and a CD4+ and CD8+ T cell. In some embodiments, the subject suffers from a cancer selected from cancer of bladder, bone, brain, breast, cervix, colon, lung, esophagus, head and neck, ovary, prostate, uterus, stomach, skin, and renal tissue. In some embodiments, the compound exhibits an ₁C50 of less than or equal to 500 nM for PTPN2 as ascertained in a phosphatase assay utilizing DiFMUP as a substrate. In some embodiments, the compound exhibits (i) an ₁C50 less than 5 nM as ascertained in a phosphatase assay utilizing DiFMUP as the substrate, and (ii) an EC₅₀ less than 10 μM in a pSTAT1 assay. In some embodiments, the compound exhibits (i) an IC₅₀ less than 5 nM as ascertained in a phosphatase assay utilizing DiFMUP as the substrate, (ii) an EC₅₀ less than 5 μM in a pSTAT1 assay, and (iii) an EC₅₀ less than 1 μM when tested in a CD25 assay. In some embodiments, expression or activity of PTPN2 is transiently downregulated by intermittent administration of the compound to the lymphoid cell. In some embodiments, a method of the present disclosure further comprises monitoring, concurrent with or subsequent to the administration of the compound and/or the lymphoid cell, one or more inflammatory biomarkers present in the subject selected from the group consisting of: antibodies, cytokines, radicals, and coagulation factors. In some embodiments, the cytokines comprise IL-1, IL-6, TNF-α, IL-10, or IL-1RR. A method of the present disclosure may further comprise administering to the subject another agent selected from the group consisting of a chemotherapeutic agent, a radioactive agent, and a checkpoint inhibitor. The additional therapeutic agent may be administered in conjunction with a compound described herein.

In certain aspects, the present disclosure provides a modified lymphoid cell comprising (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen, wherein the lymphoid cell comprises a compound described herein. In some embodiments, the compound exhibits (i) an IC₅₀ less than 5 nM as ascertained in a phosphatase assay utilizing DiFMUP as the substrate, (ii) an EC₅₀ less than 10 μM in a pSTAT1 assay, and/or (iii) an EC₅₀ less than 1 μM when tested in a CD25 assay.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise. Furthermore, use of the term “including” as well as other forms, such as “include”, “includes”, and “included”, is not limiting. The section headings used herein are for organizational purposes only and are not to be construed as limiting the subject matter described.

“About” as used herein when referring to a measurable number or value, such as an amount, duration, and the like, is meant to encompass variations of +10% of the stated number or value.

The term “Cx-y” or “Cx-Cy” when used in conjunction with a chemical moiety, such as alkyl, alkenyl, or alkynyl, is meant to include groups that contain from x to y carbons in the chain. For example, the term “Czy alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C₂₋₁₂ alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₁₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include C₃₋₁₀ monocyclic rings, C₆₋₁₂ bicyclic rings, C₇₋₁₈ polycyclic rings, C₅₋₁₂ spirocyclic rings, and C₆₋₁₂ bridged rings. Each ring of a bicyclic or polycyclic carbocycle may be selected from saturated, unsaturated, and aromatic rings. In some embodiments, the carbocycle is a C₆₋₁₂ aryl group, such as C₆₋₁₀ aryl. In some embodiments, the carbocycle is a C₃₋₁₂ cycloalkyl group. In some embodiments, the carbocycle is a C₅₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocycle. A carbocycle may comprise a fused ring, a bridged ring, a spirocyclic ring, a saturated ring, an unsaturated ring, an aromatic ring, or any combination thereof. Exemplary carbocycles include cyclopentyl, cyclohexyl, cyclohexenyl, adamantly, phenyl, indanyl, and napthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2 or 3 heteroatoms selected from O, S and N. Heterocycle may include 3- to 10-membered monocyclic rings, 6- to 12-membered bicyclic rings, 7- to 18-membered polycyclic rings, 5- to 12-membered spirocyclic rings, and 6- to 12-membered bridged rings. Each ring of a bicyclic or polycyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. A heterocycle may comprise a fused ring, a bridged ring, a spirocyclic ring, a saturated ring, an unsaturated ring, an aromatic ring, or any combination thereof. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to a 5- to 12-membered aromatic ring that comprises at least one heteroatom, such as 1, 2 or 3 heteroatoms, selected from O, S and N. As used herein, the heteroaryl ring may be selected from monocyclic or bicyclic-including fused, spirocyclic and bridged ring systems—wherein at least one of the rings in the ring system is aromatic. The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line “ [symbol] ” drawn across a bond or a dashed bond “ [symbol] ” are used interchangeably herein to denote where a bond disconnection or attachment occurs. For example, in the structure

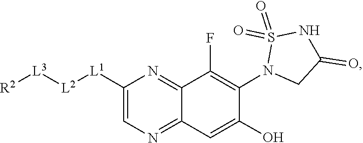

if-L¹-L²-L³-R² is cyclopropylamino as in

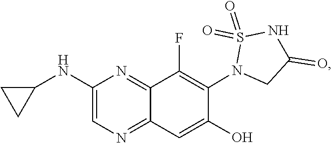 [SMILES: O=C1CN(c2c(O)cc3ncc(NC4CC4)nc3c2F)S(=O)(=O)N1]

then-L¹-L²-L³-R² may be depicted as

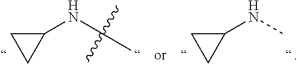

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I), is optionally substituted by one or more, such as 1, 2 or 3 substituents selected from:
halogen, oxo, —CN, C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, —CH₂-(3- to 10-membered heterocycle), —OR²², —SR²², —N(R²²)(R²³), ═NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³)—, —S(═O)(═NR²²)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, C₃₋₁₀ carbocycle, and 3- to 10-membered heterocycle, or two R²¹ are taken together with the carbon atom to which they are attached to form C₃₋₈ carbocycle or 3- to 8-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from:
halogen, oxo, —CN, C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, —CH₂-(3- to 10-membered heterocycle), —OR²², —SR²², —N(R²²)(R²³), —NR²², —C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, and —S(O)₂N(R²²)(R²³)—, wherein C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —NR²², and ═C(R²¹)_{2;}
R²¹ is independently selected at each occurrence from hydrogen, halogen, C₁₋₆ alkyl, and C₁₋₆ haloalkyl;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from halogen, oxo, —NH, —CN, —NO2, C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, —CH₂-(3- to 10-membered heterocycle), —OH, —OCH₃, —OCH₂CH₃, —NH2, —NHCH3, and —NHCH2CH₃, wherein C₁₋₆ alkyl, C₂₋₆ alkenyl, C₂₋₆ alkynyl, C₃₋₁₀ carbocycle, —CH₂-(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH (CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ^{180, 170, 35}S, ³⁶Cl, and ¹⁸F. Of particular interest are compounds of Formula (I) enriched in tritium or carbon-¹⁴, which can be used, for example, in tissue distribution studies; compounds of the disclosure enriched in deuterium-especially at a site of metabolism-resulting, for example, in compounds having greater metabolic stability; and compounds of Formula (I) enriched in a positron emitting isotope, such as ¹¹C, ¹⁸F, ¹⁵⁰ and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)- or(S)-. In some embodiments, in order to optimize the therapeutic activity of the compounds of the disclosure, e.g., to treat cancer, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis -trans or E/Z isomers (geometric isomers), tautomeric forms and topoisomeric forms of the compounds described herein are included with the scope of the disclosure unless otherwise specified.

The term “tautomer”, as used herein, refers to each of two or more isomers of a compound that exist in equilibrium and which readily interconvert. For example, one skilled in the art would understand that 1,2,3-triazole exists in two tautomeric forms:

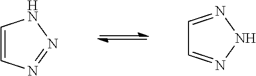 [SMILES: c1c[nH]nn1 ; c1cn[nH]n1]

Unless otherwise specified, chemical entities described herein are intended to encompass all possible tautomers, even when a structure depicts only one of them. For example, even though a single tautomer of the compound below may be depicted herein for clarity, the disclosure is intended to encompass all possible tautomers, including:

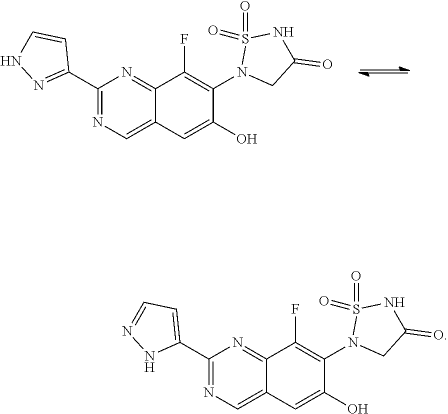 [SMILES: O=C1CN(c2c(O)cc3cnc(-c4cc[nH]n4)nc3c2F)S(=O)(=O)N1 ; O=C1CN(c2c(O)cc3cnc(-c4ccn[nH]4)nc3c2F)S(=O)(=O)N1]

The term “pharmaceutically acceptable” refers to a material that is not biologically or otherwise unacceptable when used in the subject compositions and methods. For example, the term “pharmaceutically acceptable carrier” refers to a material—such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that may be converted under physiological conditions or by solvolysis to a biologically active compound described herein (e.g., a compound of Formula (I)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The terms “administer,” “administering,” “administration,” and derivatives thereof refer to methods that may be used to enable delivery of agents or compositions to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease.

The term “systemic administration” refers to administration of agents or compositions such that the agents or compositions become distributed in a subject's body. The distribution of the agents or compositions throughout the subject's body may be an even distribution. Alternatively, the distribution may be preferential, resulting in a higher localization of the agents or compositions in one or more desired sites. A desired site may be the blood or another site that is reachable by the vascular system. Non-limiting examples of systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood. By contrast, “non-systemic administration” refers to administration of agents or compositions such that the agents or compositions are administered locally to the target site of interest of a subject's body to affect primarily a local effect.

The terms “co-administration,” “administered in combination with,” and their grammatical equivalents, encompass administration of two or more agents to a subject so that both agents and/or their metabolites can assert their respective functions. Co-administration includes simultaneous administration in separate compositions, administration at different times in separate compositions, or administration in a composition in which both agents are present.

The term “effective amount” or “therapeutically effective amount” refers to the amount of an agent that is sufficient to affect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cancer” would include preventing cancer from occurring, ameliorating cancer, suppressing cancer, and alleviating the symptoms of cancer. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., PTPN2). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject” and “patient” refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target. A “nucleotide probe” or “probe” refers to a polynucleotide used for detecting or identifying its corresponding target polynucleotide in a hybridization reaction.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The level of expression (or alternatively, the “expression level”) of a PTPN2 gene can be determined, for example, by determining the level of PTPN2 polynucleotides, polypeptides or gene products.

“Aberrantly expressed” or “aberrant expression” as applied to a nucleotide sequence (e.g., a gene) or polypeptide sequence in a subject, refers to the aberrant production of the mRNA transcribed and/or translated from the nucleotide sequence or the protein product encoded by the nucleotide sequence. A differentially expressed sequence may be overexpressed (or aberrantly high expression) or underexpressed (or aberrantly low expression) as compared to the expression level of a reference sample (i.e., a reference level). As used herein, overexpression is an increase in expression—such as by at least 1.25 fold, or alternatively, at least 1 fold, at least 2 fold, at least 3 fold, at least 4 fold, or at least 10 fold-over that detected in a reference sample. As used herein, underexpression is a reduction in expression—such as by at least 1.25 fold, or alternatively, at least 1 fold, at least 2 fold, at least 3 fold, at least 4 fold, or at least 10 fold-under that detected in a reference sample. Underexpression also encompasses absence of expression of a particular sequence as evidenced by the absence of detectable expression in a test subject when compared to a reference sample.

The term “reference level” refers to a control level used to evaluate a test level. In some examples, a reference level may be a control. For example, a biomarker may be considered to be underexpressed when the expression level of that biomarker is lower than a reference level. The reference level can be determined by a plurality of methods, provided that the resulting reference level accurately provides a level of a biomarker above which exists a first group of subjects having a different probability of exhibiting a clinically beneficial response to treatment with a PTPN2 inhibitor than that of a second group of patients having levels of the biomarker below the reference level. The reference level may be determined, for example, by measuring the level of expression of a biomarker in tumorous or non-tumorous cancer cells from the same tissue as the tissue of the cancer cells to be tested. In some examples, the reference level may be a level of a biomarker determined in vitro. A reference level may be determined by comparison of the level of a biomarker in populations of subjects having the same cancer. Two or more separate groups of subjects may be determined by identification of subsets of populations of the cohort that have the same or similar levels of a biomarker. Determination of a reference level can then be made based on a level that distinguishes these separate groups. A reference level may be a single number, equally applicable to every subject, or a reference level can vary according to specific subpopulations of subjects. For example, older men may have a different reference level than younger men for the same cancer, and women may have a different reference level than men for the same cancer. Furthermore, the reference level may be some level determined for each subject individually. For example, the reference level may be a ratio of a biomarker level in a cancer cell of a subject relative to the biomarker level in a normal cell within the same subject. In some embodiments, a reference level is a numerical range of gene expression that is obtained from a statistical sampling from a population of individuals having cancer. The sensitivity of the individuals having cancer to treatment with a PTPN2 inhibitor may be known. In certain embodiments, the reference level is derived by comparing gene expression to a control gene that is expressed in the same cellular environment at relatively stable levels (e.g. a housekeeping gene such as an actin). Comparison to a reference level may be a qualitative assessment or a quantitative determination.

The terms “determining,” “measuring,” “evaluating,” “assessing,” “assaying,” “testing,” and “analyzing” are used interchangeably herein to refer to any form of measurement and include determining if an analyte is present or not (e.g., detection).

These terms can include both quantitative and/or qualitative determinations. Assessing may be relative or absolute. A relative amount could be, for example, high, medium, or low. An absolute amount could reflect the measured strength of a signal or the translation of this signal strength into another quantitative format, such as micrograms/mL. “Detecting the presence of” can include determining the amount of something present, as well as determining whether it is present or absent.

“Signal transduction” is a process during which stimulatory or inhibitory signals are transmitted into and within a cell to elicit an intracellular response. A molecule can mediate its signaling effect via direct or indirect interaction with downstream molecules of the same pathway or related pathway(s). For instance, PTPN2 signaling can involve a host of downstream molecules including but not limited PI3-kinase and AKT.

The term “downregulating PTPN2 activity”, as used herein, refers to slowing, reducing, altering, inhibiting, as well as completely eliminating and/or preventing PTPN2 activity.

The term “effector function” refers to a specialized function of a cell. Effector function of a T-cell, for example, may be cytolytic activity or helper activity including the secretion of cytokines. Thus, the term “intracellular signaling domain” refers to the portion of a protein which transduces the effector function signal and directs the cell to perform a specialized function.

The term “autologous” refers to any material derived from the same individual to whom it is later to be re-introduced into the individual. The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include but are not limited to an MHC class I molecule, BTLA and a Toll ligand receptor, as well as OX40, CD27, CD28, CDS, ICAM-1, LFA-1 (CD11a/CD18), ICOS(CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like. The intracellular signaling domain can comprise the entire intracellular portion, or the entire native intracellular signaling domain, of the molecule from which it is derived, or a functional fragment or derivative thereof.

The terms “immune effector cell” and “effector cell” are used interchangeably here. They refer to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

The terms “immunity” and “immune response” are used herein interchangeably. As applied to a subject, they refer to the ability of the subject to elicit an immune response via their immune cells against an antigen, including without limitation tumor antigen, viral antigen, bacterial antigen, or neoantigen. As applied to a cell, the terms refer to the ability of the cell to generate a cellular response directly or indirectly against an antigen, including without limitation tumor antigen, viral antigen, bacterial antigen, or neoantigen.

The term “lymphoid cell” or “lymphoid cells” refers to any of the cells responsible for the production of immunity (or immune response) mediated by cells or antibodies and including lymphocytes, lymphoblasts, and plasma cells. Lymphoid cells include granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells (including activated T cells). In some examples, T cells include both naive and memory cells (e.g. central memory or TCM, effector memory or TEM and effector memory RA or TEMRA), effector cells (e.g. cytotoxic T cells or CTLs or Te cells), helper cells (e.g. Thl, Th2, Th3, Th9, Th7, TFH), regulatory cells (e.g. Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage.

The terms “tumor marker, “tumor antigen”, and “tumor-associated antigen” are used herein interchangeably, each referring to a molecule or fragment thereof expressed on the surface or inside of a cancer cell, or secreted or otherwise a molecule or fragment thereof derived from a cancer cell (e.g., circulating tumor DNA or circulating tumor RNA), and which is useful for the detecting a cancer cell or preferential targeting an agent to the cancer cell. A tumor antigen can be a marker expressed by both normal cells and cancer cells, e.g., a lineage marker, e.g., CD19 on B cells. A tumor antigen can be a cell surface molecule that is overexpressed or underexpressed in a cancer cell in comparison to a normal cell. A tumor antigen can also be a cell surface molecule that is inappropriately synthesized in the cancer cell, for instance, a molecule that contains deletions, additions or mutations in comparison to the molecule expressed on a normal cell. A tumor antigen can be expressed exclusively on the cell surface of a cancer cell, entirely or as a fragment (e.g., MHC/peptide), and not synthesized or expressed on the surface of a normal cell. A tumor antigen includes neoantigens encoded by tumor-specific mutated genes.

The term “transiently downregulated” as used herein generally means that a downregulation of expression or activity of a target molecule (e.g., PTPN2) is not permanent. A transient downregulation may not be a permanent downregulation. In some cases, a transient downregulation may involve downregulating (e.g., reducing) expression or activity of a target molecule for a period of time, followed by regaining at least a portion of expression or activity level of the target molecule that was previously downregulated. A transient downregulation can involve an intermittent downregulation of a target molecule (e.g., PTPN2).

The term “intermittent” is used herein to describe a process that is not continuous. An intermittent process may be followed by a break or stop. A plurality of intermittent processes may involve alternatively starting and stopping a same process or different processes. In some embodiments, the term “intermittent dosing regimen” as used here refers to a dosing regimen that comprises administering a pharmaceutical composition, followed by a rest period.

The term “side effect” as used herein refers to any complication, unwanted, or pathological outcome of a therapy (e.g., a cell therapy, an immunotherapy, etc.) that occurs in addition to or in place of a desired treatment outcome of the therapy. Examples of a side effect may include, but are not limited to, (i) off-target cell toxicity, (ii) on-target off-tumor toxicity, and/or (iii) autoimmunity (e.g., chronic autoimmunity). In an example, a side effect of a cell therapy involving a T-cell receptor fusion protein (TFP) and/or a chimeric antigen receptor (CAR) may include a graft-versus-host disease. In another example, a side effect of a cell therapy involving a TFP and/or a CAR may include death of a cell configured to express the TFP and/or the CAR.

Other examples of a side effect of a cell therapy may include, but are not limited to, disorders mediated by phagocytic cells, which includes macrophages and neutrophil granulocytes (Polymorphonuclear leukocytes, PMNs) and/or T cells. Examples include inflammatory skin diseases including psoriasis; responses associated with inflammatory bowel disease (such as Crohn's disease and ulcerative colitis); adult respiratory distress syndrome; dermatitis; CNS inflammatory disorders such as multiple sclerosis; uveitic disorders; allergic conditions such as eczema and asthma and other conditions involving infiltration of T cells and chronic inflammatory responses; skin hypersensitivity reactions (including poison ivy and poison oak); autoimmune diseases such as rheumatoid arthritis, systemic lupus erythematosus (SLE), diabetes mellitus, multiple sclerosis, Raynaud's syndrome, autoimmune thyroiditis, Sjogren's syndrome, juvenile onset diabetes, and immune responses associated with delayed hypersensitivity mediated by cytokines and T-lymphocytes typically found in tuberculosis, sarcoidosis, polymyositis, granulomatosis and vasculitis; pernicious anemia; multiple organ injury syndrome secondary to septicaemia or trauma; autoimmune haemolytic anemia; myethemia gravis; antigen-antibody complex mediated diseases; and/or all types of transplantation rejection, including graft vs. host or host vs. graft disease.

The term “efficacy” of a treatment or method, as used herein, can be measured based on changes in the course of disease or condition in response to such treatment or method. For example, the efficacy of a treatment or method of the present disclosure may be measured by its impact on signs or symptoms of a disease or condition of a subject, e.g., a tumor or cancer of the subject. A response may be achieved when a subject having the disease or condition experiences partial or total alleviation of the disease or condition, or reduction of one or more symptoms of the disease or condition. In an example, a response is achieved when a subject suffering from a tumor exhibits a reduction in the tumor size after the treatment or method, as provided in the present disclosure. In some examples, the efficacy may be measured by assessing cancer cell death, reduction of tumor (e.g., as evidenced by tumor size reduction), and/or inhibition of tumor growth, progression, and dissemination.

An “antigen” is a moiety or molecule that contains an epitope, and, as such, also specifically binds to an antibody. An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or VHH), a heavy chain found in sharks (V-NAR domain), a single domain antibody (sdAb, e.g., “nanobody”) that comprises a single antigen-binding domain, Fv, Fd, Fab, Fab′, F(ab′)₂, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH₃)₂, (scFv-CH₃)₂, ((scFv)₂-CH₃+CH₃), ((scFv)₂-CH₃) or (scFv-CH₃-scFv)₂), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Compounds

Compounds of Formula (I) disclosed herein-including the compounds of Formula (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), and (I-C2a)-or pharmaceutically acceptable salts or solvates thereof, are PTPN2 inhibitors and have a wide range of applications in therapeutics, diagnostics, and other biomedical research.

In certain aspects, the present disclosure provides a compound of Formula (I):

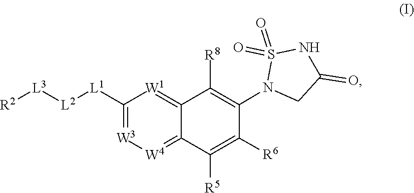

or a pharmaceutically acceptable salt or solvate thereof, wherein:
W¹ is N, W³ is N, and W⁴ is C(R⁴); W¹ is N, W³ is C(R³), and W⁴ is N; or W¹ is C(R¹), W³ is N, and W⁴ is N;
R¹, R³, R⁴, R⁵, R⁶, and R⁸ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
L¹ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—;
L² is selected from C₁₋₆ alkylene, C₂₋₆ alkenylene, C₂₋₆ alkynylene, —C₀₋₃ alkylene-C_{3.8} carbocycle-, and —C₀₋₃ alkylene-(3- to 8-membered heterocycle)-, each of which is optionally substituted with one, two, or three R²⁰;
L³ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—;
R² is selected from hydrogen, halogen, —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R₁₃ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, —C₀₋₃ alkyl-C₃₋₁₀ carbocycle, —C₀₋₃ alkyl-(3- to 10-membered heterocycle), —OR²², —SR²², —N(R²²)(R²³), —NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N)R²²)(R²³), —N(R²⁴)C(O)R²⁵, —S(O)₂R²⁵, —S(O)₂N(R²²)(R²³)—, —S(═O)(═NR²²)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², —C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, C₃₋₁₀ carbocycle, and 3- to 10-membered heterocycle, or two R²¹ are taken together with the carbon atom to which they are attached to form C₃₋₈ carbocycle or 3- to 8-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

In some embodiments, the compound of Formula (I) is a compound of Formula (I-1) or (I-2):

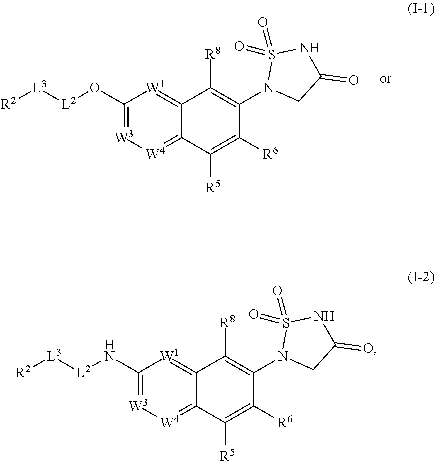

or a pharmaceutically acceptable salt or solvate thereof.

In some embodiments, the compound of Formula (I) is a compound of Formula (Ia), such as a compound of Formula (I-1a) or (I-2a):

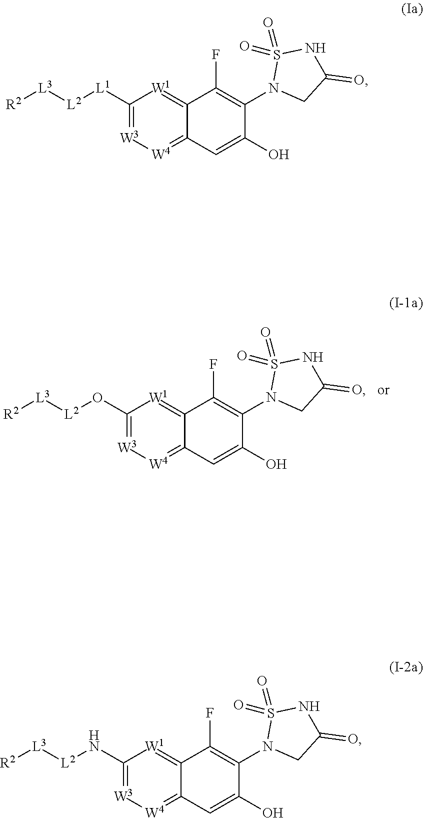

or a pharmaceutically acceptable salt or solvate thereof.

In some embodiments, for a compound of Formula (I), (I-1), (I-2), (Ia), (I-1a), or (I-2a), R¹, R³, R⁴, R⁵, R⁶, and R⁸ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R¹, R³, R⁴, R⁵, R⁶, and R⁸ are independently selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6-membered heterocycle are optionally substituted with one, two, or three R²⁰. In some embodiments, R¹, R³, R⁴, R⁵, R⁶, and R⁸ are independently selected from hydrogen, halogen, C₁₋₆ alkyl, C₃₋₆ carbocycle, 3- to 6-membered heterocycle, —OR¹², and —N(R¹²)(R¹³), wherein C₁₋₆ alkyl, C₃₋₆ carbocycle, and 3- to 6-membered heterocycle are optionally substituted with one, two, or three R²⁰. In some embodiments, R¹, R³, R⁴, R⁵, R⁶, and R⁸ are independently selected from hydrogen, halogen, —OR¹², and C₁₋₆ alkyl, wherein C₁₋₆ alkyl is optionally substituted with one, two, or three R²⁰. In some embodiments, R¹, R³, R⁴, R⁵, R⁶, and R⁸ are independently selected from hydrogen, halogen, C₁₋₃ alkyl, C₁₋₃ haloalkyl, C₃₋₆ carbocycle, —OH, —OCH₃, —NH₂, and —NHCH₃. In some embodiments, R¹, R³, R⁴, R⁵, R⁶, and R⁸ are independently selected from hydrogen, halogen, and —OH. In some embodiments, R¹, R³, R⁴, and R⁵ are each hydrogen; R⁶ is —OH, and R⁸ is halogen. In some embodiments, R¹, R³, R⁴, and R⁵ are each hydrogen; R⁶ is —OH, and R⁸ is fluorine.

In some embodiments, for a compound of Formula (I), (I-1), (I-2), (Ia), (I-1a), or (I-2a), R¹, R³, and R⁴ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R¹, R³, and R⁴ are independently selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6-membered heterocycle are optionally substituted with one, two, or three R²⁰. In some embodiments, R¹, R³, and R⁴ are independently selected from hydrogen, halogen, C₁₋₃ alkyl, C₁₋₃ haloalkyl, C₃₋₆ carbocycle, —OH, —OCH₃, —NH₂, and —NHCH₃. In some embodiments, R¹, R³, and R⁴ are each hydrogen.

In some embodiments, for a compound of Formula (I), (I-1), (I-2), (Ia), (I-1a), or (I-2a), R¹ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R¹ is selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6- membered heterocycle are optionally substituted with one, two, or three R²⁰. In some embodiments, R¹ is selected from hydrogen, halogen, C₁₋₃ alkyl, C₁₋₃ haloalkyl, C₃₋₆ carbocycle, —OH, —OCH₃, —NH₂, and —NHCH₃. In some embodiments, R¹ is selected from hydrogen, chlorine, and fluorine. In some embodiments, R¹ is hydrogen.

In some embodiments, for a compound of Formula (I), (I-1), (I-2), (Ia), (I-1a), or (I-2a), R³ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R³ is selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6- membered heterocycle are optionally substituted with one, two, or three R²⁰. In some embodiments, R³ is selected from hydrogen, halogen, C₁₋₃ alkyl, C₁₋₃ haloalkyl, C₃₋₆ carbocycle, —OH, —OCH₃, —NH₂, and —NHCH₃. In some embodiments, R³ is selected from hydrogen, —OH, and —NH₂. In some embodiments, R³ is hydrogen.

In some embodiments, for a compound of Formula (I), (I-1), (I-2), (Ia), (I-1a), or (I-2a), R⁴ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R⁴ is selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6- membered heterocycle are optionally substituted with one, two, or three R²⁰. In some embodiments, R⁴ is selected from hydrogen, halogen, C₁₋₃ alkyl, C₁₋₃ haloalkyl, C₃₋₆ carbocycle, —OH, —OCH₃, —NH₂, and —NHCH₃. In some embodiments, R⁴ is hydrogen.

In some embodiments, for a compound of Formula (I), (I-1), (I-2), (Ia), (I-1a), or (I-2a), W¹ is N, W³ is N, and W⁴ is C(R⁴). In some embodiments, W¹ is N, W³ is N, W⁴ is C(R⁴), R⁵ is hydrogen, R⁶ is —OH, and R⁸ is fluorine. In some embodiments, W¹ is N, W³ is N, and W⁴ is CH. In some embodiments, W¹ is N, W³ is N, W⁴ is CH, R⁵ is hydrogen, R⁶ is —OH, and R⁸ is fluorine. In some embodiments, W¹ is N, W³ is C(R³), and W⁴ is N. In some embodiments, W¹ is N, W³ is C(R³), W⁴ is N, R⁵ is hydrogen, R⁶ is —OH, and R⁸ is fluorine. In some embodiments, W¹ is N, W³ is CH, and W⁴ is N. In some embodiments, W¹ is N, W³ is CH, W⁴ is N, R⁵ is hydrogen, R⁶ is —OH, and R⁸ is fluorine. In some embodiments, W¹ is C(R¹), W³ is N, and W⁴ is N. In some embodiments, W¹ is C(R¹), W³ is N, W⁴ is N, R⁵ is hydrogen, R⁶ is —OH, and R⁸ is fluorine. In some embodiments, W¹ is CH, W³ is N, and W⁴ is N. In some embodiments, W¹ is CH, W³ is N, W⁴ is N, R⁵ is hydrogen, R⁶ is —OH, and R⁸ is fluorine.

In some embodiments, the compound of Formula (I) is a compound of Formula (I-A), such as a compound of Formula (I-A1) or (I-A2):

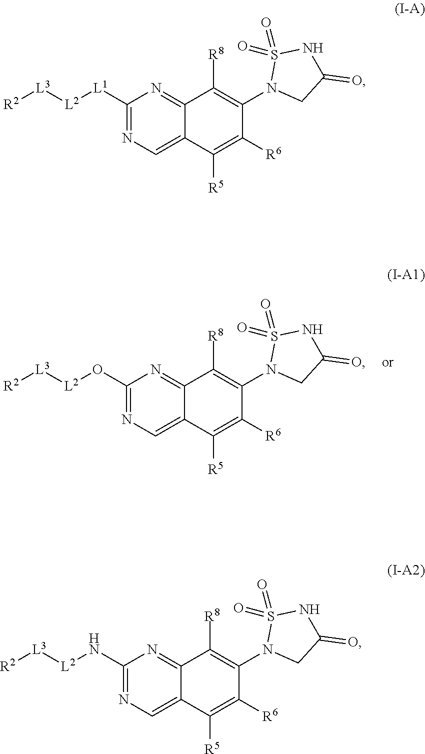

or a pharmaceutically acceptable salt or solvate thereof.

In some embodiments, the compound of Formula (I) is a compound of Formula (I-B), such as a compound of Formula (I-B1) or (I-B2):

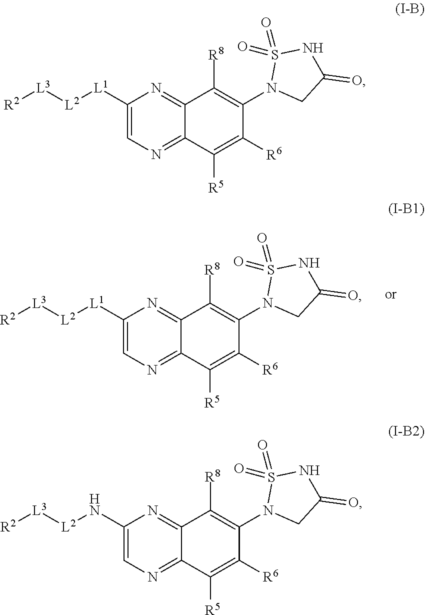

or a pharmaceutically acceptable salt or solvate thereof.

In some embodiments, the compound of Formula (I) is a compound of Formula (I-C), such as a compound of Formula (I-C1) or (I-C2):

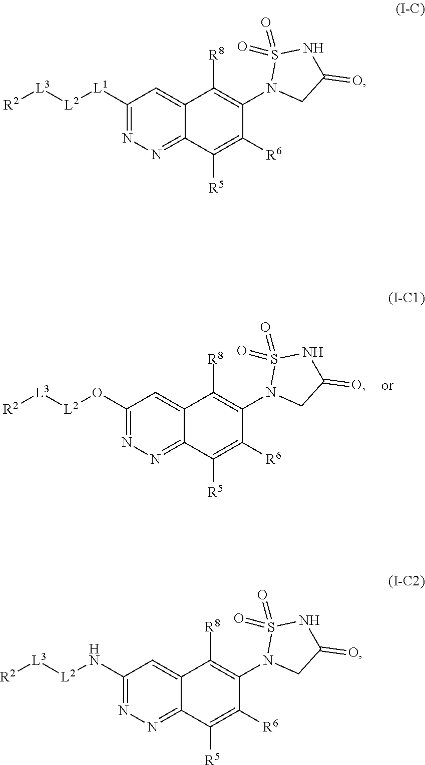

or a pharmaceutically acceptable salt or solvate thereof.

In some embodiments, for a compound of Formula (I), (I-1), (I-2), (I-A), (I-A1), (I-A2), (I-B), (I-B1), (I-B2), (I-C), (I-C1), or (I-C2), R⁵, R⁶, and R⁸ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R⁵, R⁶, and R⁸ are independently selected from hydrogen, halogen, C₁₋₆ alkyl, C₃₋₆ carbocycle, 3- to 6-membered heterocycle, —OR¹², and —N(R¹²)(R¹³), wherein C₁₋₆ alkyl, C₃₋₆ carbocycle, and 3- to 6-membered heterocycle are optionally substituted with one, two, or three R²⁰. In some embodiments, R⁵, R⁶, and R⁸ are independently selected from hydrogen, halogen, —OR¹², and C₁₋₆ alkyl, wherein C₁₋₆ alkyl is optionally substituted with one, two, or three R²⁰. In some embodiments, R⁵, R⁶, and R⁸ are independently selected from hydrogen, halogen, and —OH. In some embodiments, R⁵, R⁶, and R⁸ are independently selected from hydrogen, fluorine, and —OH. In some embodiments, R⁵ is hydrogen, R⁶ is selected from halogen, —OR¹², and C₁₋₆ alkyl, wherein C₁₋₆ alkyl is optionally substituted with one, two, or three R²⁰; and R⁸ is halogen. In some embodiments, R⁵ is hydrogen, R⁶ is —OH, and R⁸ is halogen. In some embodiments, R⁵ is hydrogen, R⁶ is —OH, and R⁸ is fluorine. In some embodiments, R⁵ is —OH, R⁶ is hydrogen, and R⁸ is fluorine.

In some embodiments, for a compound of Formula (I), (I-1), (I-2), (I-A), (I-A1), (I-A2), (I-B), (I-B1), (I-B2), (I-C), (I-C1), or (I-C2), R⁵ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R⁵ is selected from hydrogen, halogen, C₁₋₆ alkyl, C₃₋₆ carbocycle, 3- to 6-membered heterocycle, —OR¹², and —N(R¹²)(R¹³), wherein C₁₋₆ alkyl, C₃₋₆ carbocycle, and 3- to 6-membered heterocycle are optionally substituted with one, two, or three R²⁰. In some embodiments, R⁵ is selected from hydrogen, halogen, and —OH. In some embodiments, R⁵ is hydrogen.

In some embodiments, for a compound of Formula (I), (I-1), (I-2), (I-A), (I-A1), (I-A2), (I-B), (I-B1), (I-B2), (I-C), (I-C1), or (I-C2), R⁶ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R⁶ is selected from halogen, —OR¹², and C₁₋₆ alkyl, wherein C₁₋₆ alkyl is optionally substituted with one, two, or three R²⁰. In some embodiments, R⁶ is selected from hydrogen, halogen, and —OH. In some embodiments, R⁶ is —OH.

In some embodiments, for a compound of Formula (I), (I-1), (I-2), (I-A), (I-A1), (I-A2), (I-B), (I-B1), (I-B2), (I-C), (I-C1), or (I-C2), R⁸ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —N(R¹⁴)S(O)₂R¹⁵, —C(O)R¹², —S(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R⁸ is selected from halogen, —OR¹², and C₁₋₆ alkyl, wherein C₁₋₆ alkyl is optionally substituted with one, two, or three R²⁰. In some embodiments, R⁸ is selected from hydrogen, halogen, and —OH. In some embodiments, R⁸ is halogen, such as fluorine.

In some embodiments, the compound of Formula (I) is a compound of Formula (I-Aa), such as a compound of Formula (I-A1a) or (I-A2a):

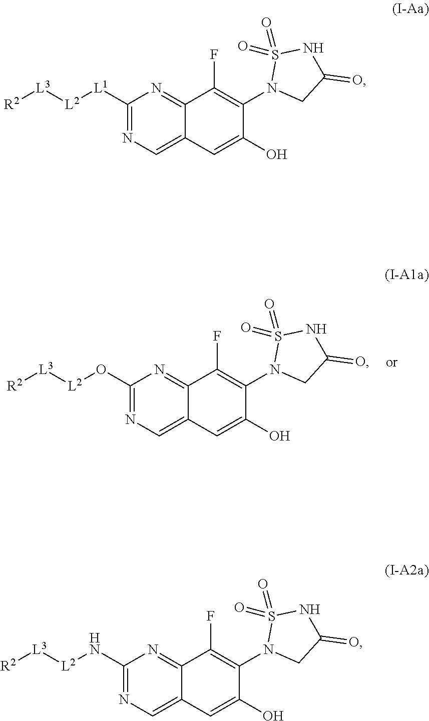

or a pharmaceutically acceptable salt or solvate thereof.

In some embodiments, the compound of Formula (I) is a compound of Formula (I-Ba), such as a compound of Formula (I-B1a) or (I-B2a):

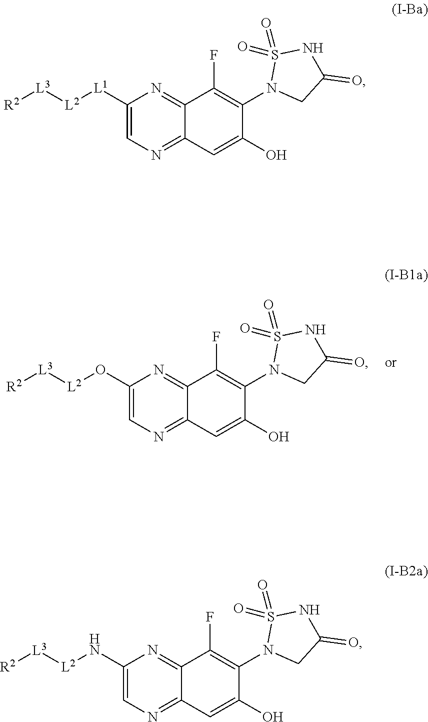

or a pharmaceutically acceptable salt or solvate thereof.

In some embodiments, the compound of Formula (I) is a compound of Formula (I-Ca), such as a compound of Formula (I-C1a) or (I-C2a):

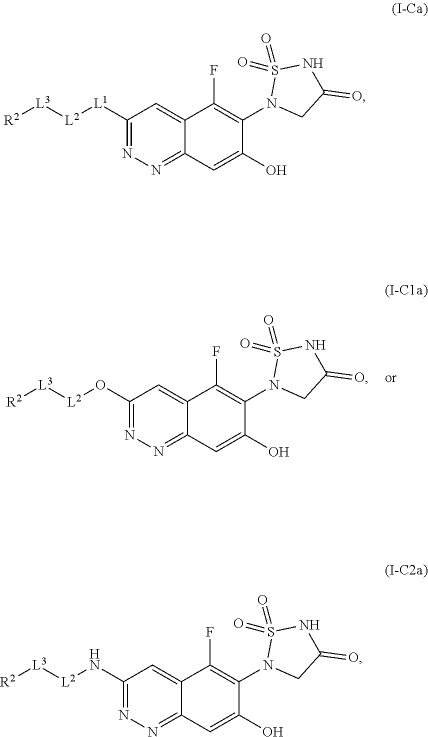

or a pharmaceutically acceptable salt or solvate thereof.

In some embodiments, for a compound of Formula (I), (Ia), (I-A), (I-Aa), (I-B), (I-Ba), (I-C), or (I-Ca), L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵; and L³ is selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—. In some embodiments, L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L¹ is selected from —O—, and —N(R¹²)—; L² is selected from C₁₋₆ alkylene, —C₀₋₃ alkylene-C_{3.8} carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L¹ is absent; L² is 3- to 8-membered heterocycle, optionally substituted with one, two, or three R²⁰; and L³ is selected from absent and —S(O)₂—.

In some embodiments, for a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—. In some embodiments, L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵; and L³ is selected from absent, —N(R¹²)—, —C(O)O—, —OC(O)—, and —S(O)₂—. In some embodiments, L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L² is selected from C₁₋₆ alkylene, —C₀₋₃ alkylene-C₃₋₈ carbocycle-, and —C₀₋₃ alkylene-(3to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, L² is 3- to 8-membered heterocycle, optionally substituted with one, two, or three R²⁰; and L³ is selected from absent and —S(O)₂—

In some embodiments, for a compound of Formula (I), (Ia), (I-A), (I-Aa), (I-B), (I-Ba), (I-C), or (I-Ca), L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵; L³ is selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C_{3.8} carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, L¹ is selected from —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, and C₁₋₆ haloalkyl. In some embodiments, L¹ is absent; L² is 3- to 8-membered heterocycle, optionally substituted with one, two, or three R²⁰; L³ is selected from absent and —S(O)₂—; and R² is selected from hydrogen, halogen, —CN, C₁₋₆ alkyl, and C₁₋₆ haloalkyl.

In some embodiments, for a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵; L³ is selected from absent, —N(R¹²)—, —C(O)O—, —OC(O)—, and —S(O)₂—; and R² is selected from hydrogen, halogen, —CN, C₁₋₆ alkyl, C_{3.8} carbocycle, and 3- to 8-membered heterocycle, wherein C₁₋₆ alkyl, C_{3.8} carbocycle, and 3- to 8-membered heterocycle are optionally substituted with one, two, or three R²⁰. In some embodiments, L² is selected from C₁₋₆ alkylene, —C₀₋₃ alkylene-C_{3.8} carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, and C₁₋₆ haloalkyl. In some embodiments, L² is 3- to 8-membered heterocycle, optionally substituted with one, two, or three R²⁰; L³ is selected from absent and —S(O)₂—; and R² is selected from hydrogen, halogen, —CN, C₁₋₆ alkyl, and C₁₋₆ haloalkyl.

In some embodiments, for a compound of Formula (I), (Ia), (I-A), (I-Aa), (I-B), (I-Ba), (I-C), or (I-Ca), L¹ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—. In some embodiments, L¹ is selected from absent, —O—, —S—, —N(R¹²)—, —C(O)O—, —N(R¹²)C(O)N(R¹²)—, —N(R¹²)S(O)₂—, —C(O)—, —OC(O)—, —C(O)N(R¹²)—, —N(R¹²)C(O)—, —S(O)₂—, —S(O)₂N(R¹²)—, —N(R¹²)S(O)₂N(R¹²)—, —P(O)(OR¹²)—, and —P(O)(R¹²)—. In some embodiments, L¹ is selected from absent, —O—, —S-, —N(R¹²)—, —C(NR¹²)—, —N(R¹²)S(O)₂—, —S(O)—, —S(O)₂—, and —S(O)₂N(R¹²)—. In some embodiments, L¹ is selected from absent, —O—, and —N(R¹²)—. In some embodiments, L¹ is selected from —O- and —N(R¹²)—. In some embodiments, L¹ is absent. In some embodiments, L¹ is —O—. In some embodiments, L¹ is —N(R¹²)—.

In some embodiments, for a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰. In some embodiments, L² is selected from C₁₋₆ alkylene, —C₀₋₃ alkylene-C₃₋₆ carbocycle-, and —C₀₋₃ alkylene-(3- to 6-membered heterocycle)-, each of which is optionally substituted with one, two, or three substituents independently selected from halogen, oxo, —CN, —OH, and —S(O)₂R²⁵. In some embodiments, L² is C₁-6 alkylene, such as C₁₋₃ alkylene, optionally substituted with one, two, or three R²⁰; such as optionally substituted with one, two, or three substituents independently selected from halogen, oxo, —CN, —OH, and —S(O)₂R²⁵. In some embodiments, L² is C₁-6 alkylene, such as C₁₋₃ alkylene. In some embodiments, L² is —C₀₋₃ alkylene-C₃₋₆ carbocycle-, optionally substituted with one, two, or three R²⁰; such as optionally substituted with one, two, or three substituents independently selected from halogen, oxo, —CN, —OH, and —S(O)₂R²⁵. In some embodiments, L² is —C₀₋₃ alkylene-C₃₋₆ carbocycle-. In some embodiments, L² is-C₀₋₃ alkylene-(3- to 6-membered heterocycle)-, optionally substituted with one, two, or three R²⁰; such as optionally substituted with one, two, or three substituents independently selected from halogen, oxo, —CN, —OH, and —S(O)₂R²⁵. In some embodiments, L² is-C₀₋₃ alkylene-(3- to 6-membered heterocycle)-. In some embodiments, the C₃₋₈ carbocycle of L² is selected from C₃₋₈ monocyclic cycloalkyl, C₅₋₈ monocyclic cycloalkenyl, and C₆ monocyclic aryl, each of which is optionally substituted with one, two, or three R²⁰; and the 3- to 8-membered heterocycle of L² is selected from 3- to 8-membered monocyclic heterocycloalkyl, 5- to 8-membered monocyclic heterocycloalkenyl, and 5- to 6-membered monocyclic heteroaryl, each of which is optionally substituted with one, two, or three R²⁰. In some embodiments, the C₃₋₆ carbocycle of L² is selected from C₃₋₆ monocyclic cycloalkyl, C₅₋₆ monocyclic cycloalkenyl, and C₆ monocyclic aryl, each of which is optionally substituted with one, two, or three R²⁰; and the 3- to 6-membered heterocycle of L² is selected from 3- to 6-membered monocyclic heterocycloalkyl, 5- to 6-membered monocyclic heterocycloalkenyl, and 5- to 6-membered monocyclic heteroaryl, each of which is optionally substituted with one, two, or three R²⁰. In some embodiments, carbocycle is selected from cycloalkyl, cycloalkenyl, and aryl, each of which is optionally substituted with one, two, or three R²⁰; and heterocycle is selected from heterocycloalkyl, heterocycloalkenyl, and heteroaryl, each of which is optionally substituted with one, two, or three R²⁰. In some embodiments, carbocycle is selected from cycloalkyl and aryl, each of which is optionally substituted with one, two, or three R²⁰; and heterocycle is selected from heterocycloalkyl, heterocycloalkenyl, and heteroaryl, each of which is optionally substituted with one, two, or three R²⁰. In some embodiments, the carbocycle is unsaturated, such as a carbocycle comprising one double bond. In some embodiments, the carbocycle is saturated. In some embodiments, the carbocycle is aromatic. In some embodiments, the heterocycle is unsaturated, such as a heterocycle comprising one double bond. In some embodiments, the heterocycle is saturated. In some embodiments, the heterocycle is aromatic.

In some embodiments, for a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), L³ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—. In some embodiments, L³ is selected from absent, —O—, —S—, —N(R¹²)—, —C(O)O—, —N(R¹²)C(O)N(R¹²)—, —N(R¹²)S(O)₂—, —C(O)—, —OC(O)—, —C(O)N(R¹²)—, —N(R¹²)C(O)—, —S(O)₂—, —S(O)₂N(R¹²)—, —N(R¹²)S(O)₂N(R¹²)—, —P(O)(OR¹²)—, and —P(O)(R¹²)—. In some embodiments, L³ is selected from absent, —O—, —S—, —N(R¹²)—, —C(O)O—, —N(R¹²)C(O)N(R¹²)—, —N(R¹²)S(O)₂—, —S(O)—, —OC(O)—, —C(O)N(R¹²)—, —N(R¹²)C(O)—, —S(O)₂—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, and —N(R¹²)S(O)N(R¹²)—. In some embodiments, L³ is selected from absent, —N(R¹²)—, —C(O)O—, —OC(O)—, and —S(O)₂—. In some embodiments, L³ is absent. In some embodiments, L³ is —N(R¹²)—. In some embodiments, L³ is selected from —C(O)O— and —OC(O)—. In some embodiments, L³ is-S(O)₂—.

In some embodiments, for a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), R² is selected from hydrogen, halogen, —CN, C₁₋₆ alkyl, C₂₋₆ alkenyl, C₂₋₆ alkynyl, C₃₋₈ carbocycle, and 3- to 8-membered heterocycle, wherein C₁₋₆ alkyl, C₂₋₆ alkenyl, C₂₋₆ alkynyl, C₃₋₈ carbocycle, and 3- to 8-membered heterocycle are optionally substituted with one, two, or three R²⁰. In some embodiments, R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₆ alkyl, —C₀₋₃ alkyl-C₃₋₆ carbocycle, —C₀₋₃ alkyl-(3- to 6-membered heterocycle), —OR²², —N(R²²)(R²³), ═NR²², ═C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—, wherein C₁₋₆ alkyl, —C₀₋₃ alkyl-C₃₋₆ carbocycle, and —C₀₋₃ alkyl-(3- to 6- membered heterocycle) are optionally substituted with one, two, or three substituents independently selected from halogen, oxo, —CN, C₁₋₆ alkyl, —OR²², —N(R²²)(R²³), ═NR²²,═C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—. In some embodiments, R² is selected from hydrogen, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₆ alkyl, —C₀₋₃ alkyl-C₃₋₆ carbocycle, —C₀₋₃ alkyl-(3- to 6-membered heterocycle), —OR²², —N(R²²)(R²³), ═NR²², —C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—, wherein C₁₋₆ alkyl, —C₀₋₃ alkyl-C₃₋₆ carbocycle, and —C₀₋₃ alkyl-(3- to 6-membered heterocycle) are optionally substituted with one, two, or three substituents independently selected from halogen, oxo, —CN, C₁₋₆ alkyl, —OR²², —N(R²²)(R²³), ═NR²², ═C(R²¹)₂, —C(O)OR²², —OC(O)N(R²²)(R²³), —N(R²⁴)C(O)OR²⁵, —N(R²⁴)S(O)₂R²⁵, —OC(O)R²⁵, —C(O)N(R²²)(R²³), —N(R²⁴)C(O)R²⁵, —S(O)₂R²⁵, and —S(O)₂N(R²²)(R²³)—. In some embodiments, R² is selected from hydrogen, halogen, —CN, C₁₋₆ alkyl, C₃₋₈ carbocycle, and 3- to 8--31-Attorney Docket No. 56690-760.301 membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, R² is selected from hydrogen, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, R² is hydrogen. In some embodiments, R² is halogen, such as fluorine. In some embodiments, R² is-CN. In some embodiments, R² is C₁-6 alkyl. In some embodiments, the C₃₋₈ carbocycle of R² is selected from C₃₋₈ monocyclic cycloalkyl, C₅₋₈ monocyclic cycloalkenyl, and C₆ monocyclic aryl, each of which is optionally substituted with one, two, or three R²⁰; and the 3- to 8-membered heterocycle of R² is selected from 3- to 8-membered monocyclic heterocycloalkyl, 5to 8-membered monocyclic heterocycloalkenyl, and 5- to 6-membered monocyclic heteroaryl, each of which is optionally substituted with one, two, or three R²⁰. In some embodiments, the C₃₋₆ carbocycle of R² is selected from C₃₋₆ monocyclic cycloalkyl, C₅₋₆ monocyclic cycloalkenyl, and C₆ monocyclic aryl, each of which is optionally substituted with one, two, or three R²⁰; and the 3- to 6-membered heterocycle of R² is selected from 3- to 6-membered monocyclic heterocycloalkyl, 5- to 6-membered monocyclic heterocycloalkenyl, and 5- to 6-membered monocyclic heteroaryl, each of which is optionally substituted with one, two, or three R²⁰. In some embodiments, R² is C₃₋₆ cycloalkyl, such as cyclopropyl. In some embodiments, carbocycle is selected from cycloalkyl, cycloalkenyl, and aryl, each of which is optionally substituted with one, two, or three R²⁰; and heterocycle is selected from heterocycloalkyl, heterocycloalkenyl, and heteroaryl, each of which is optionally substituted with one, two, or three R²⁰. In some embodiments, carbocycle is selected from cycloalkyl and aryl, each of which is optionally substituted with one, two, or three R²⁰; and heterocycle is selected from heterocycloalkyl, heterocycloalkenyl, and heteroaryl, each of which is optionally substituted with one, two, or three R²⁰. In some embodiments, the carbocycle is unsaturated, such as a carbocycle comprising one double bond. In some embodiments, the carbocycle is saturated. In some embodiments, the carbocycle is aromatic. In some embodiments, the heterocycle is unsaturated, such as a heterocycle comprising one double bond. In some embodiments, the heterocycle is saturated. In some embodiments, the heterocycle is aromatic.

In some embodiments, for a compound of Formula (I), (Ia), (I-A), (I-Aa), (I-B), (I-Ba), (I-C), or (I-Ca), —L¹-L²-L³-R² is selected from

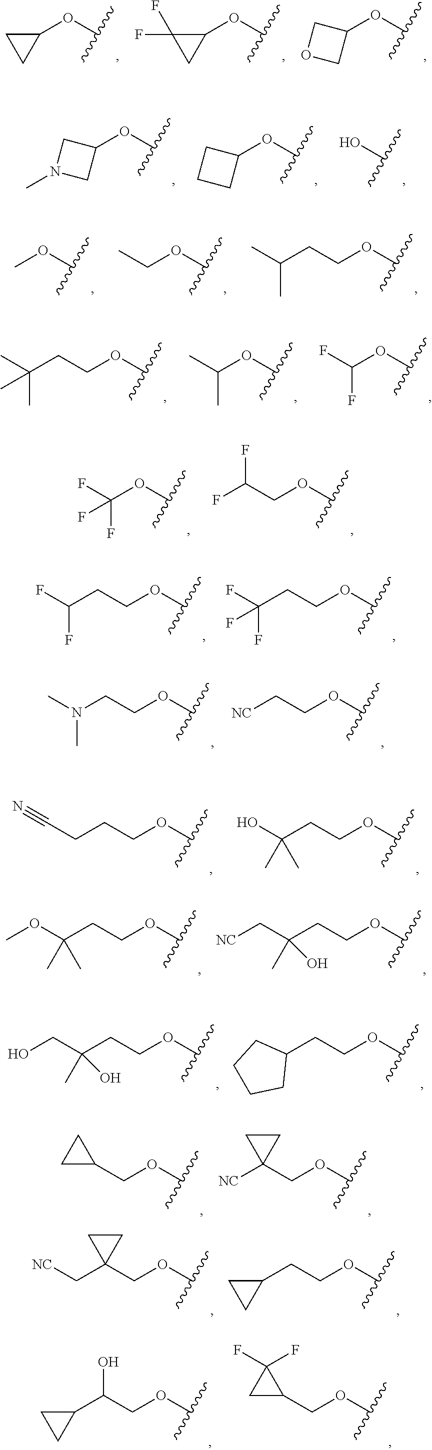

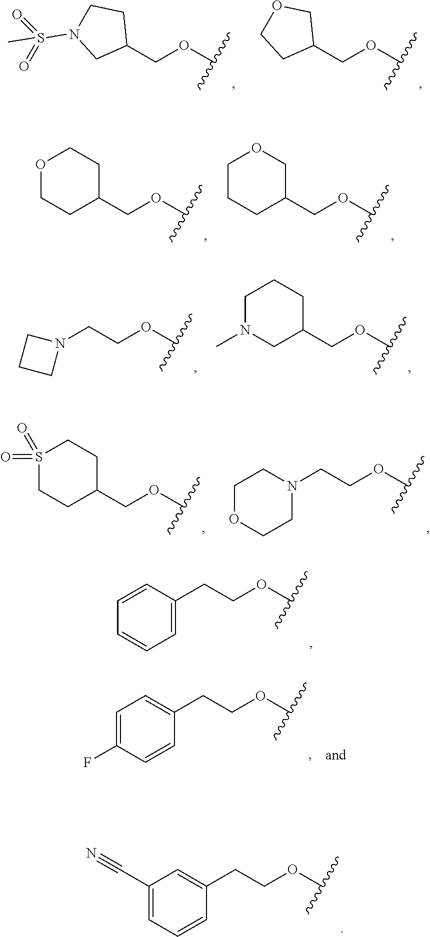

In some embodiments, for a compound of Formula (I), (Ia), (I-A), (I-Aa), (I-B), (I-Ba), (I-C), or (I-Ca), —L¹-L²-L³-R² is selected from

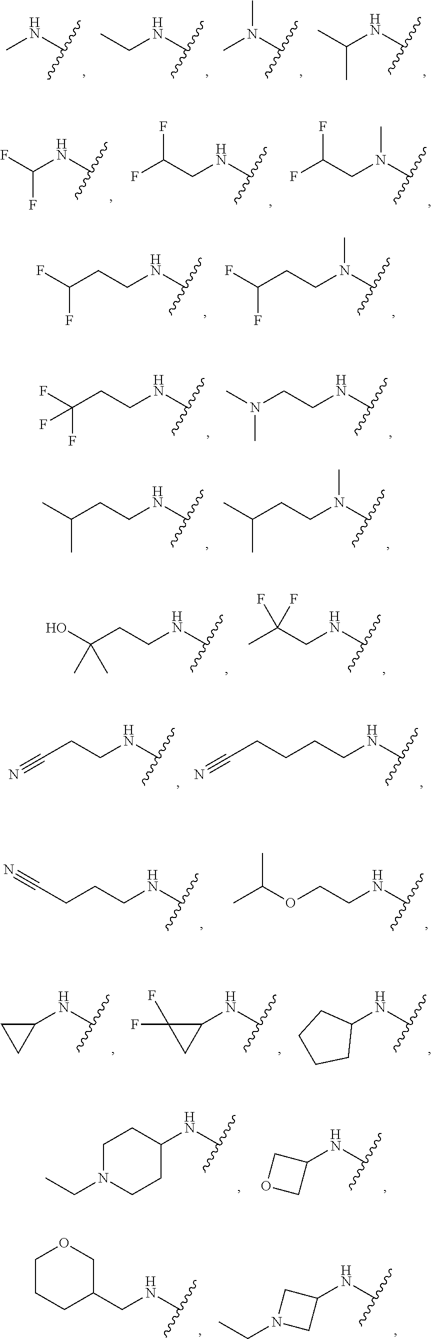

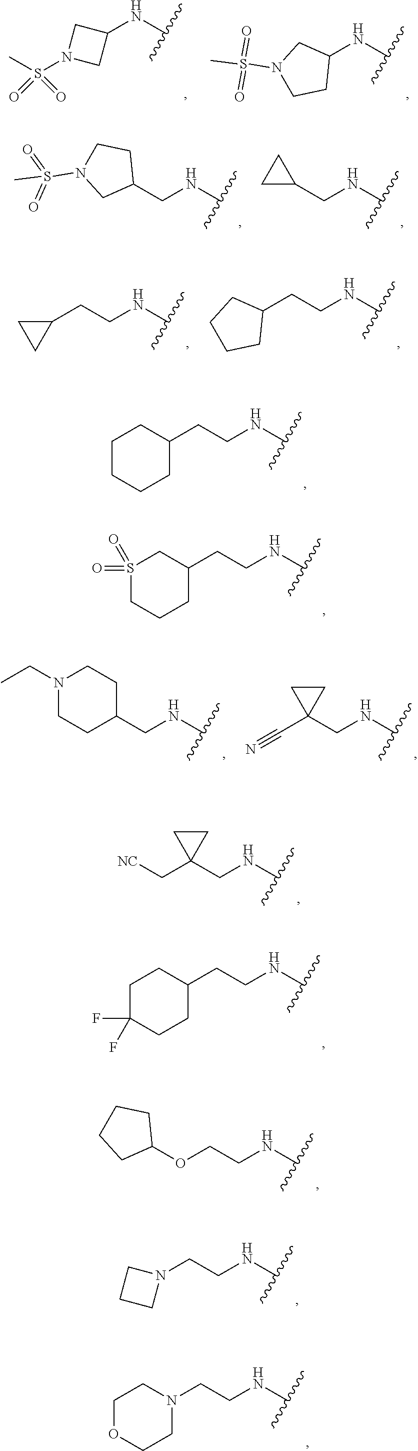

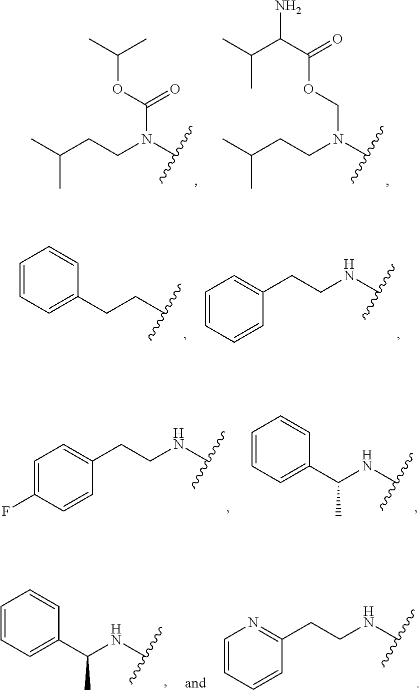

In some embodiments, for a compound of Formula (I), (Ia), (I-A), (I-Aa), (I-B), (I-Ba), (I-C), or (I-Ca), —L¹-L²-L³-R² is selected from

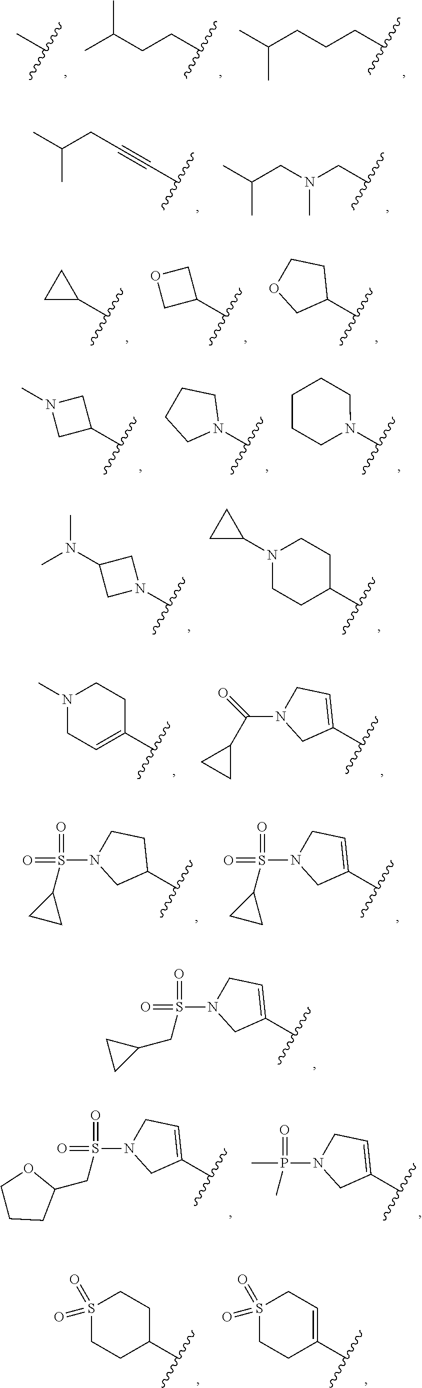

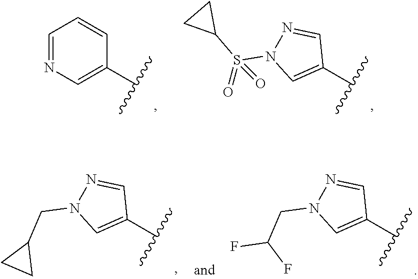 [SMILES: *c1cccnc1 ; *c1cnn(S(=O)(=O)C2CC2)c1 ; *c1cnn(CC2CC2)c1 ; *c1cnn(CC(F)F)c1]

In some embodiments, for a compound of Formula (I), (Ia), (I-A), (I-Aa), (I-B), (I-Ba), (I-C), or (I-Ca), —L¹-L²-L³-R² is selected from

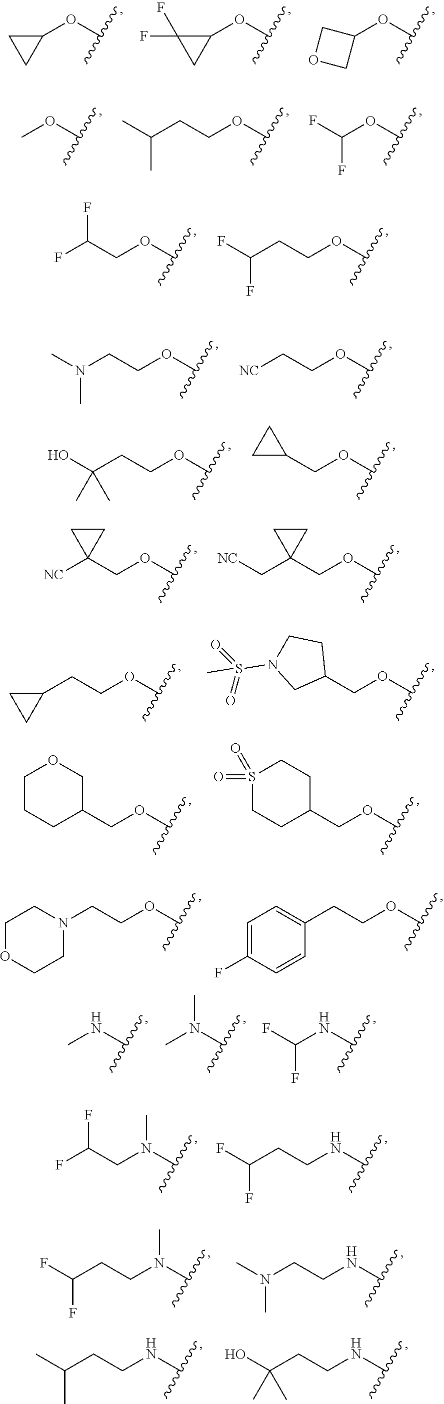

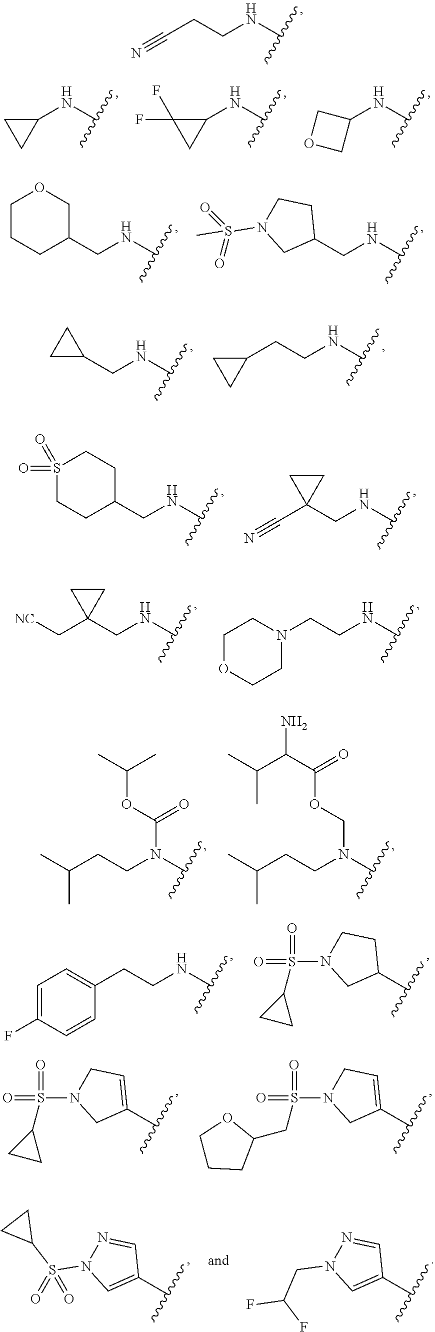

In some embodiments, for a compound of Formula (I), R¹, R³, R⁴, R⁵, R⁶, and R⁸ are independently selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6-membered heterocycle are optionally substituted with one, two, or three R²⁰; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, R¹, R³, R⁴, R⁵, R⁶, and R⁸ are independently selected from hydrogen, halogen, —OR¹², and C₁₋₆ alkyl, wherein C₁₋₆ alkyl is optionally substituted with one, two, or three R²⁰; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, R¹, R³, R⁴, R⁵, R⁶, and R⁸ are independently selected from hydrogen, halogen, and —OH; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, R¹, R³, R⁴, and R⁵ are each hydrogen; R⁶ is —OH; R⁸ is halogen; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, R¹, R³, R⁴, and R⁵ are each hydrogen; R⁶ is —OH; R⁸ is fluorine; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—.

In some embodiments, for a compound of Formula (I), R¹, R³, R⁴, R⁵, R⁶, and R⁸ are independently selected from hydrogen, halogen, C₁₋₆ alkyl, C₃₋₆ carbocycle, 3- to 6-membered heterocycle, —OR¹², —N(R¹²)(R¹³), —S(O)R¹⁵, —C(O)N(R¹²)(R¹³), —N(R¹⁴)C(O)R¹⁵, and —S(O)₂R¹⁵, wherein C₁₋₆ alkyl, C₃₋₆ carbocycle, and 3- to 6-membered heterocycle are optionally substituted with one, two, or three R²⁰; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, R¹, R³, R⁴, R⁵, R⁶, and R⁸ are independently selected from hydrogen, halogen, —OR¹², and C₁₋₆ alkyl, wherein C₁₋₆ alkyl is optionally substituted with one, two, or three R²⁰; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, R¹, R³, R⁴, R⁵, R⁶, and R⁸ are independently selected from hydrogen, halogen, and —OH; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, R¹, R³, R⁴, and R⁵ are each hydrogen; R⁶ is —OH; R⁸ is halogen; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂. In some embodiments, R¹, R³, R⁴, and R⁵ are each hydrogen; R⁶ is —OH; R⁸ is fluorine; L¹ is selected from absent, —O—, —S—, —N(R¹²)—, —C(NR¹²)—, —N(R¹²)S(O)₂—, —S(O)—, —S(O)₂—, and —S(O)₂N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle, —C₀₋₃ alkyl-(3- to 6-membered heterocycle), —OH, and —NH₂.

In some embodiments, for a compound of Formula (I), (I-A), (I-B), or (I-C), R⁵ is hydrogen; R⁶ is —OH; R⁸ is halogen; L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and L³ is selected from absent, —N(R¹²)—, —C(O)O—, —OC(O)—, and —S(O)₂—. In some embodiments, R⁵ is hydrogen; R⁶ is —OH; R⁸ is halogen; L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰. In some embodiments, R⁵ is hydrogen; R⁶ is —OH; R⁸ is halogen; L¹ is selected from absent, —O—, and —N(R¹²)—; L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; L³ is selected from absent, —N(R¹²)—, —C(O)O—, —OC(O)—, and —S(O)₂—; and R² is selected from hydrogen, halogen, —CN, C₁₋₆ alkyl, CMar. 8, carbocycle, and 3- to 8-membered heterocycle, wherein C₁₋₆ alkyl, C₃₋₈ carbocycle, and 3- to 8-membered heterocycle are optionally substituted with one, two, or three substituents independently selected from halogen, oxo, —CN, C₁₋₃ alkyl, —C₀₋₃ alkyl-C₃₋₆ carbocycle,-C₀₋₃ alkyl-(3- to 6-membered heterocycle), —OH, and —NH₂.

In certain aspects, the present disclosure provides a compound selected from:

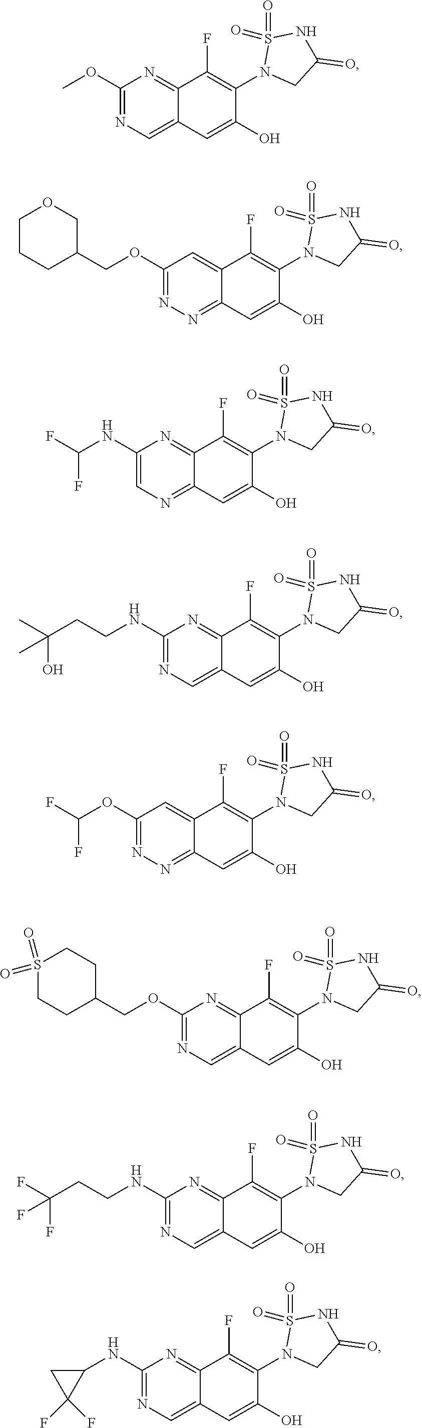

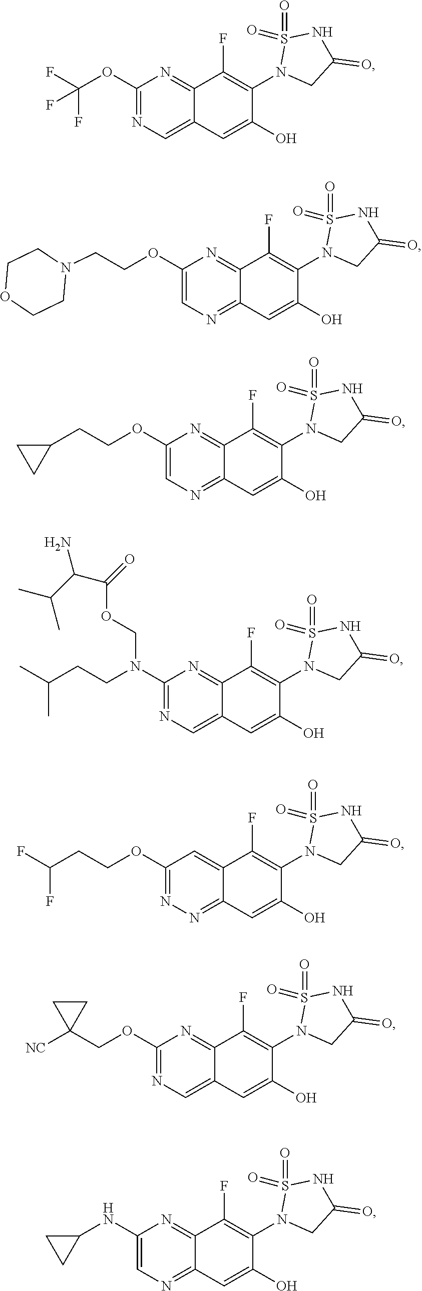

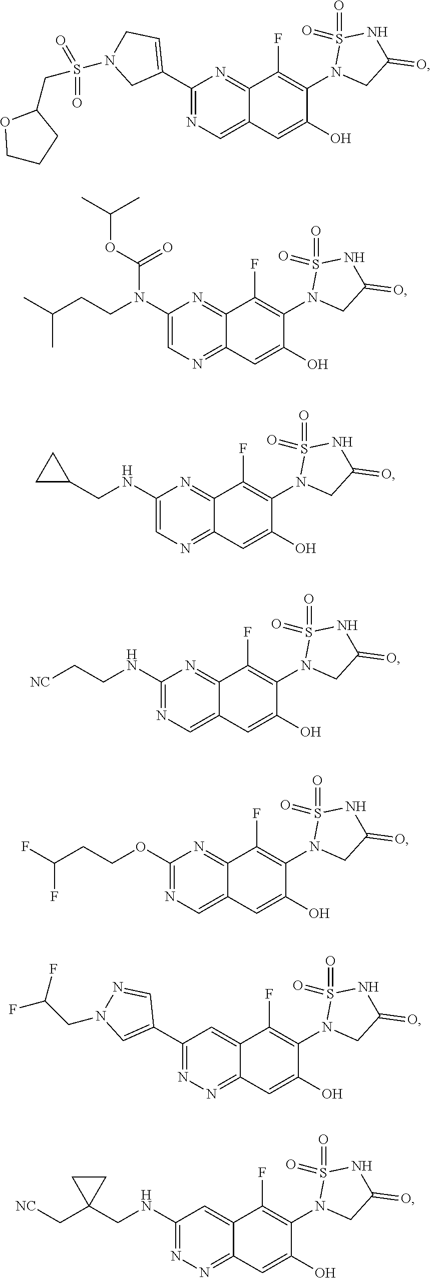

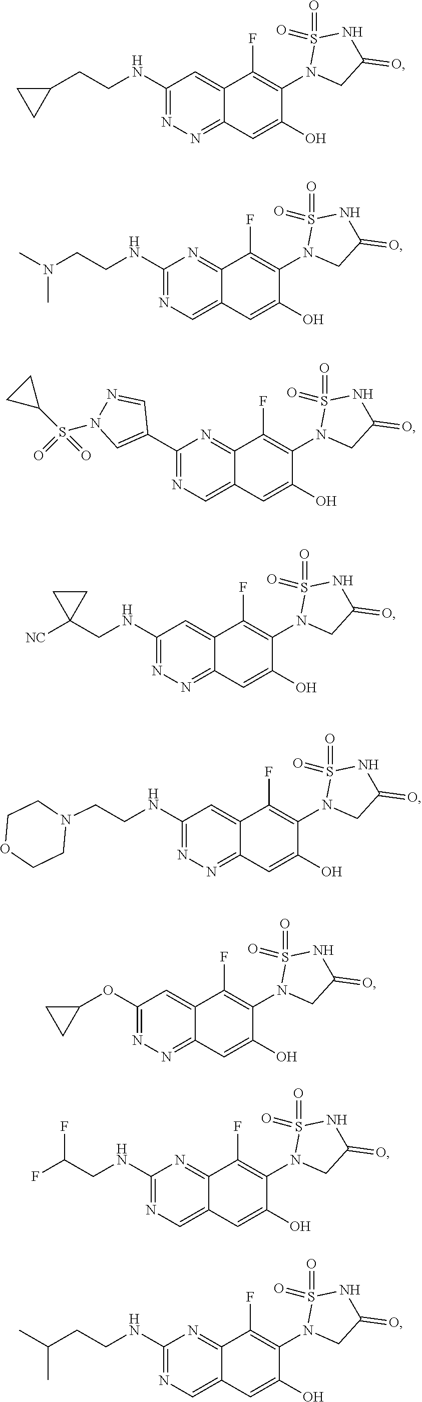

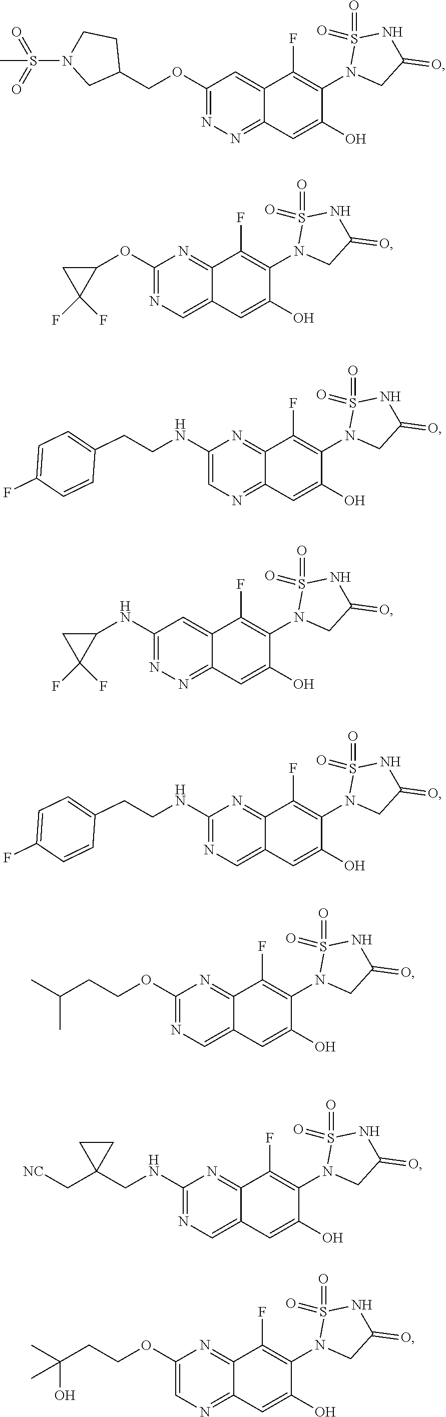

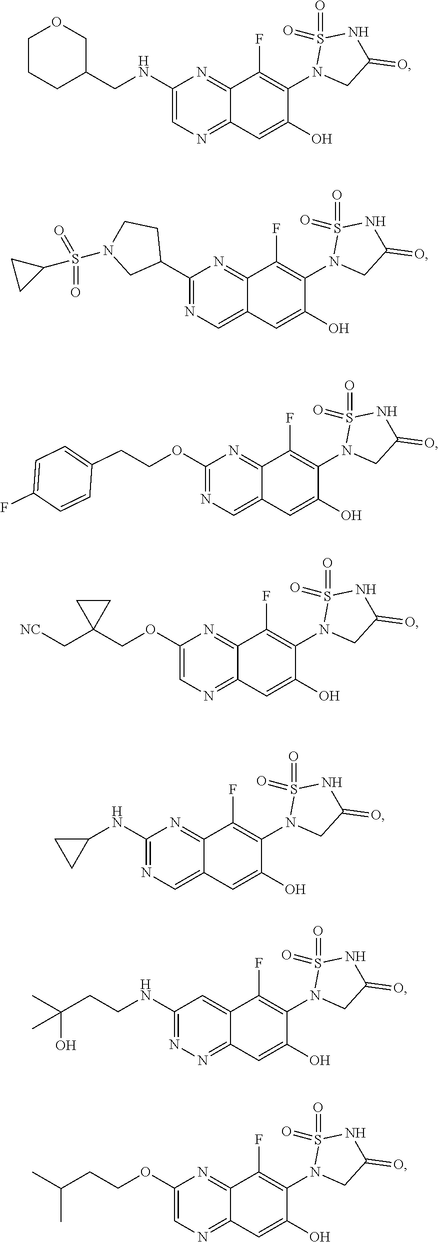

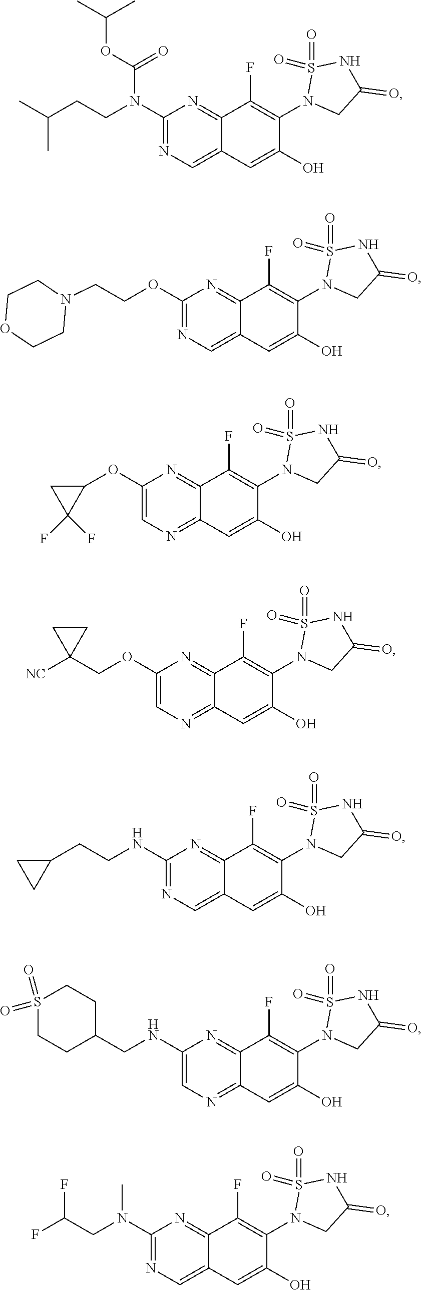

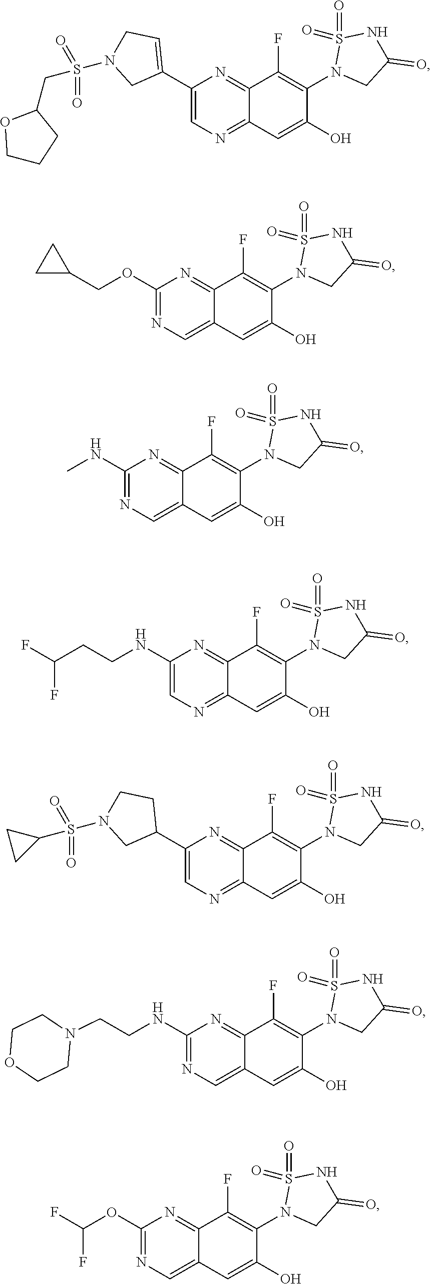

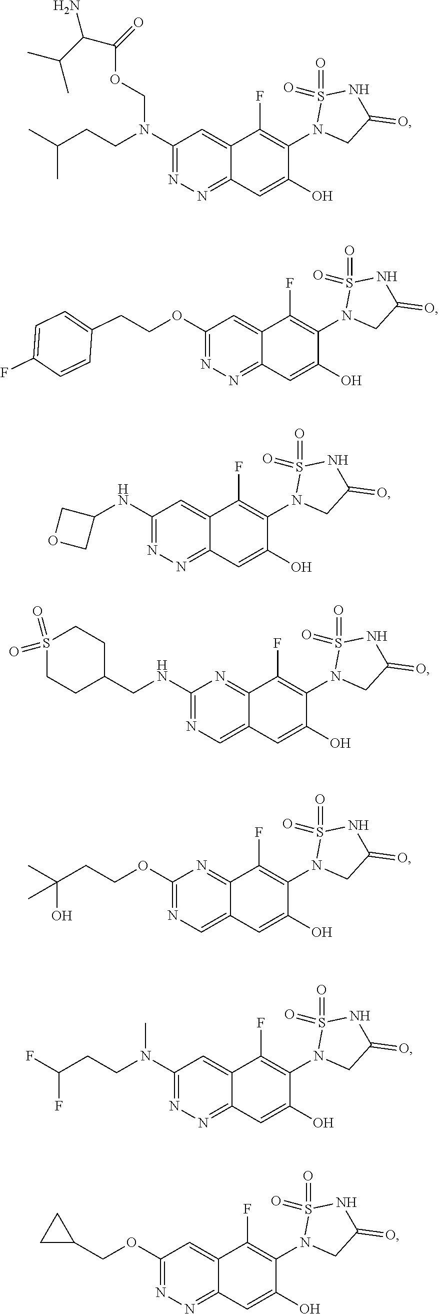

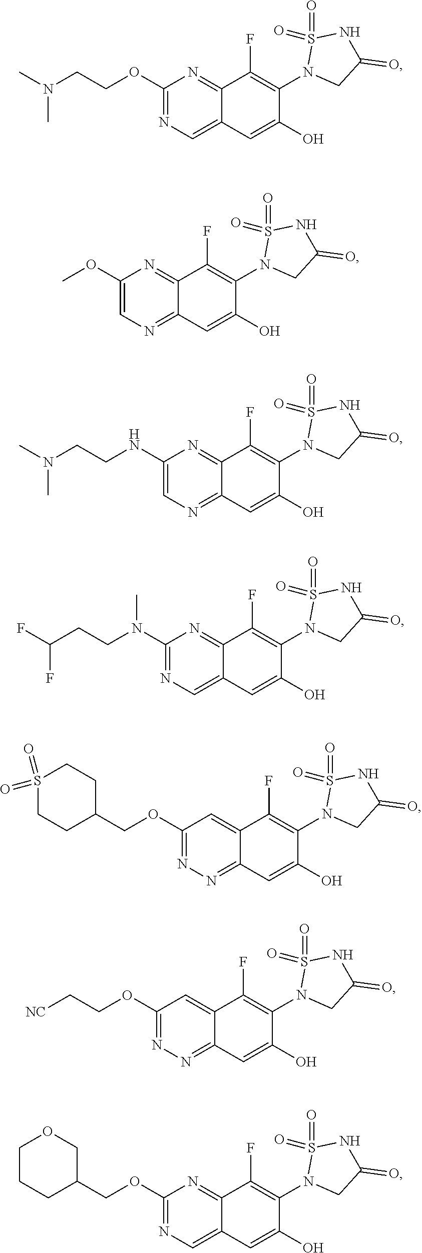

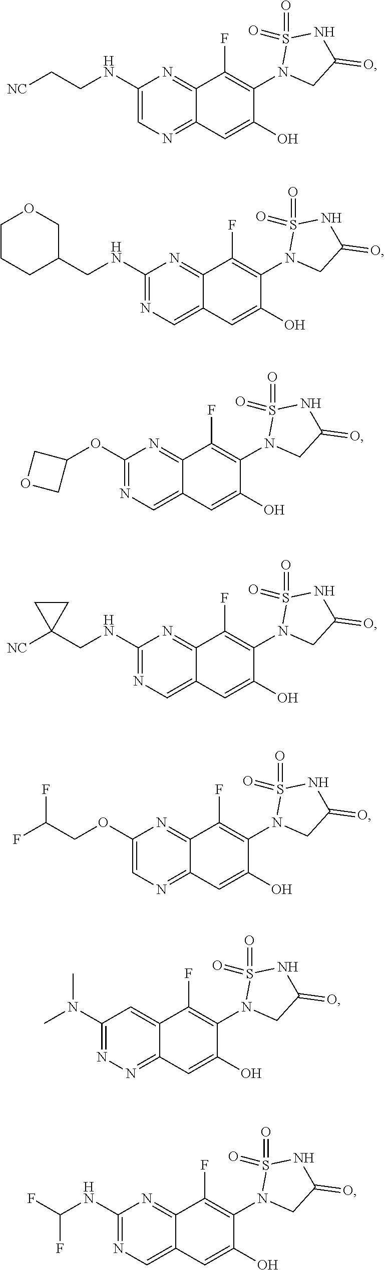

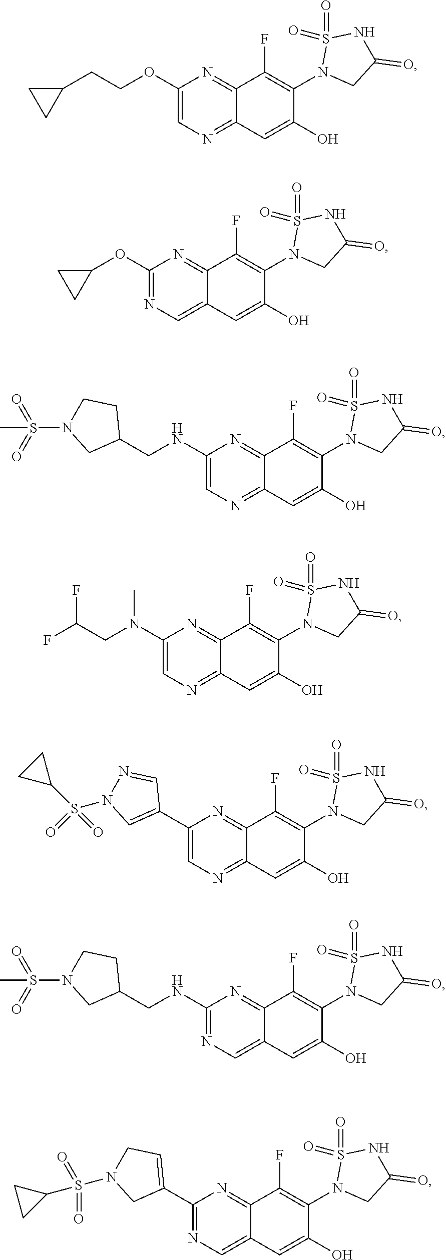

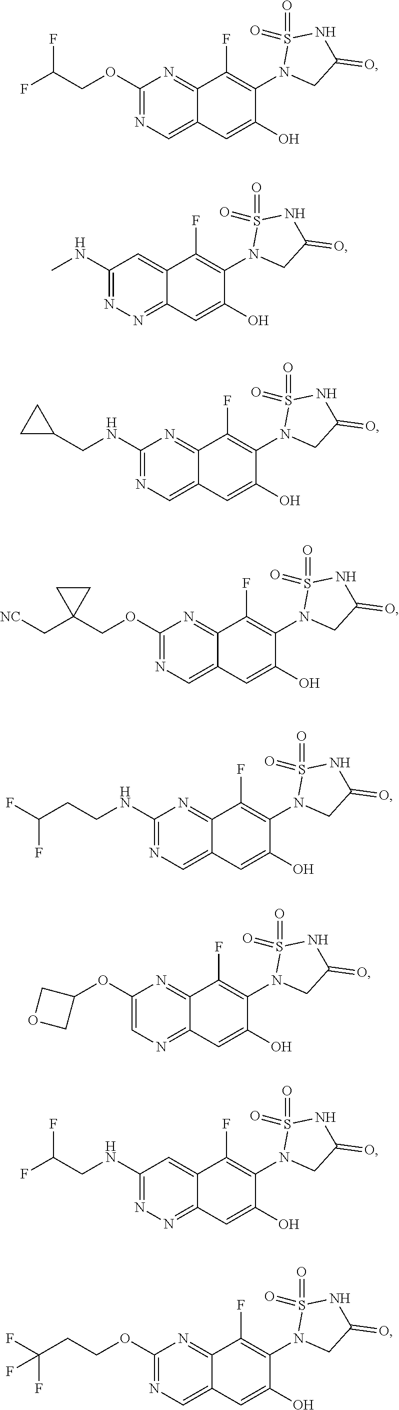

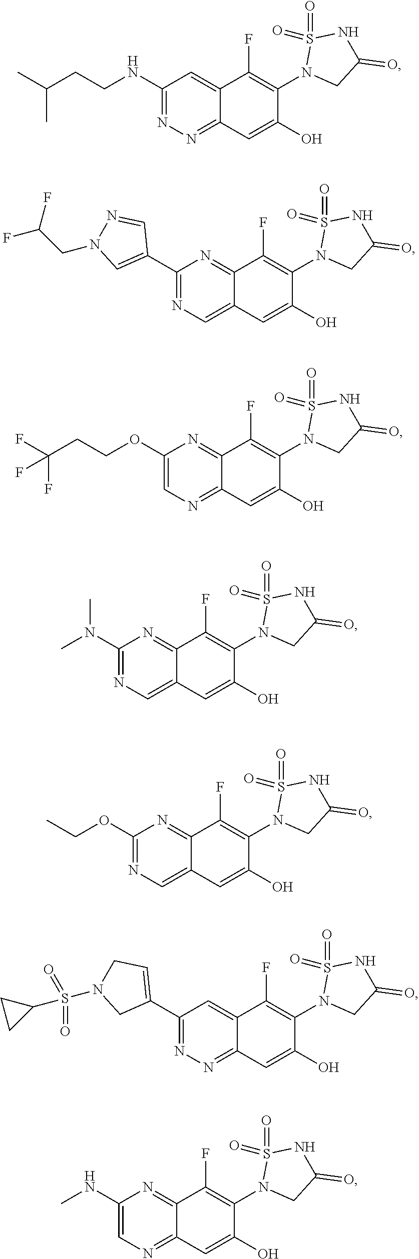

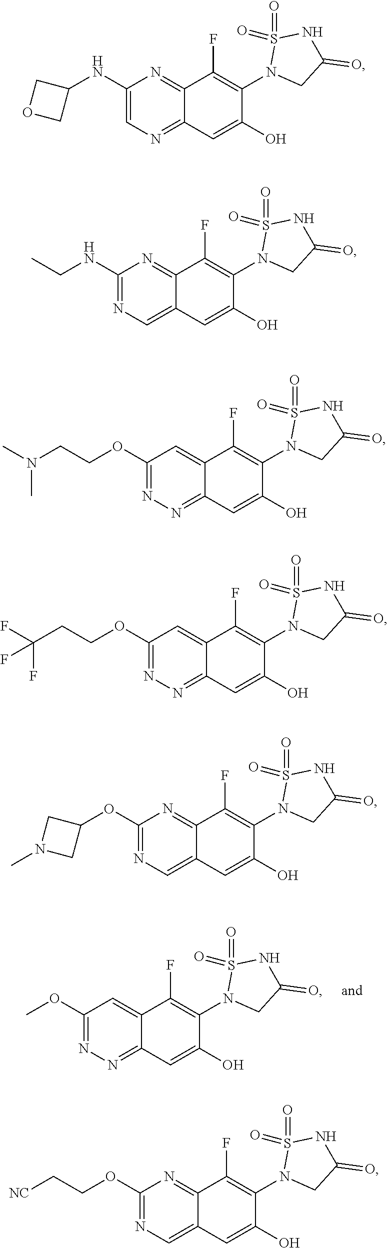

or a pharmaceutically acceptable salt or solvate thereof.

In some embodiments, a compound of Formula (I), such as a compound of Formula (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

In certain aspects, the present disclosure provides a compound of the formula B-L^{BE}-E wherein:
B is a monovalent form of a compound described herein;
L^{BE} is a covalent linker bonded to B and E; and
E is a monovalent form of a degradation enhancer.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In some embodiments, the degradation enhancer is capable of binding to an E3 ubiquitin ligase protein or a protein complex comprising an E3 ubiquitin ligase protein. In some embodiments, the degradation enhancer is capable of binding to an E2 ubiquitin-conjugating enzyme. In some embodiments, the degradation enhancer is capable of binding to a protein complex comprising an E2 ubiquitin-conjugating enzyme and an E3 ubiquitin ligase protein.

In some embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C(anaphase-promoting complex/cyclosome), CRBN(cereblon), CULA-RBX1-DDB1-CRBN(CRLACRBN) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885. In some embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R¹, UBE2R2, UBE2S, UBE2T, UBE2U, UBE2V1, UBE2V2, UBE2W, UBE2Z, ATG3, BIRC6, and UFC1. In some embodiments, the degradation enhancer is a compound described in Ishida and Ciulli, SLAS Discovery 2021, Vol. 25 (4) 484-502, which is incorporated by reference in its entirety for any purpose, for example VH032, VH101, VH298, thalidomide, bestatin, methyl bestatin, nutlin, idasanutlin, bardoxolone, bardoxolone methyl, indisulam (E7070), E7820, chloroquinoxaline sulfonamide (CQS), nimbolide, KB02, ASTX660, lenalidomide, or pomalidomide.

In some embodiments, the degradation enhancer is a compound described in US20180050021, WO2016146985, WO2018189554, WO2018119441, WO2018140809, WO2018119448, WO2018119357, WO2018118598, WO2018102067, WO201898280, WO201889736, WO201881530, WO201871606, WO201864589, WO201852949, WO2017223452, WO2017204445, WO2017197055, WO2017197046, WO2017180417, WO2017176958, WO201711371, WO2018226542, WO2018223909, WO2018189554, WO2016169989, WO2016146985, CN105085620B, CN106543185B, U.S. Pat. Nos. 10,040,804, 9,938,302, 10,144,745, 10,145,848, 9,938,264, 9,632,089, 9,821,068, 9,758,522, 9,500,653, 9,765,019, 8,507,488, 8,299,057, US20180298027, US20180215731, US20170065719, US20170037004, US20160272639, US20150291562, or US20140356322, each of which is incorporated by reference in its entirety for any purpose.

In some embodiments, L^{BE} is-L^{BE1}-L^{BE2}-L^{BE3}-L^{BE4}-L^{BE5}-;
L^{BE1}, L^{BE2}, L^{BE3}, L^{BE4}, and L^{BE5} are independently a bond, —O—, —N(R¹²)—, —C(O)—, —N(R¹²)C(O)—, —C(O)N(R¹²)—, —S—, —S(O)₂—, —S(O)—, —S(O)₂N(R¹²)—, —S(O)N(R¹²)—, —N(R¹²)S(O)—, —N(R¹²)S(O)₂—, C₁₋₆ alkylene, (—O—C₁₋₆ alkyl)_{z}-, (—C₁₋₆ alkyl-O)_{z}—, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁-11 heteroarylene, wherein C₁₋₆ alkylene, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁-11 heteroarylene are optionally substituted with one, two, or three R²⁰; and wherein each C₁₋₆ alkyl of (—O—C₁₋₆ alkyl)_{z}— and (—C₁₋₆ alkyl-O)_{z}— is optionally substituted with one, two, or three R²⁰; and
is independently an integer from 0 to 10.

In some embodiments, L^{BE} is —(O—C₂ alkyl)_{z}- and z is an integer from 1 to 10. In some embodiments, L^{BE} is —(C₂ alkyl-O—)_{z}— and z is an integer from 1 to 10. In some embodiments, L^{BE} is —(CH₂)_{zz1}L^{BE2}(CH₂O)_{zz2}—, wherein L^{BE2} is a bond, a 5- or 6-membered heterocyclene, phenylene, —C₂₋₄ alkynylene, —SO₂— or —NH—; and zz1 and zz2 are independently an integer from 0 to 10. In some embodiments, L^{BE} is —(CH₂)_{zz1} (CH₂₀)_{zz2}—, wherein zz1 and zz2 are each independently an integer from 0 to 10. In some embodiments, L^{BE} is a PEG linker (e.g., divalent linker of 1 to 10 ethylene glycol subunits). In some embodiments, E is a monovalent form of a compound selected from

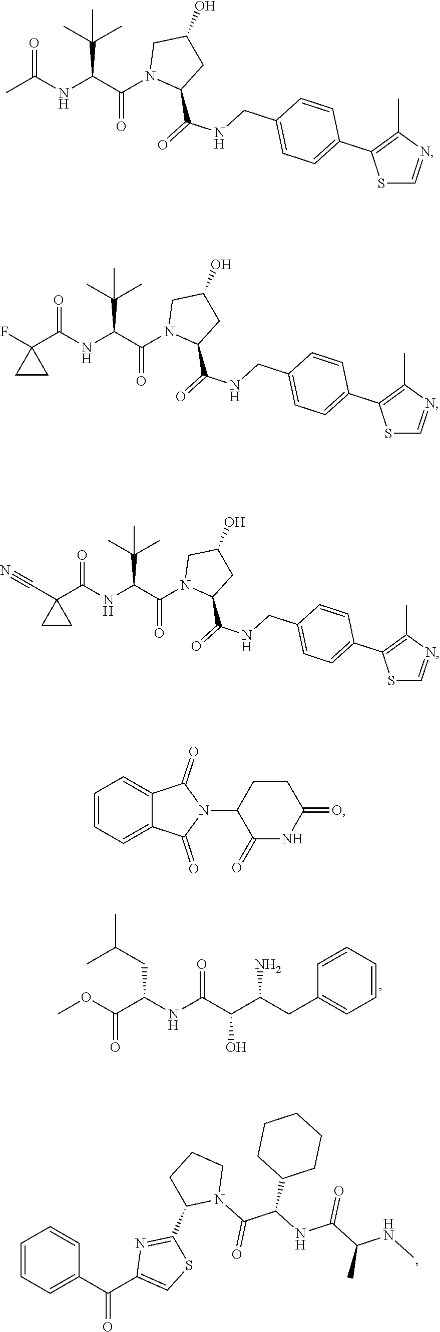 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1 ; O=C1CCC(N2C(=O)c3ccccc3C2=O)C(=O)N1 ; COC(=O)[C@H](CC(C)C)NC(=O)[C@@H](O)[C@H](N)Cc1ccccc1 ; CN[C@@H](C)C(=O)N[C@H](C(=O)N1CCC[C@H]1c1nc(C(=O)c2ccccc2)cs1)C1CCCCC1]

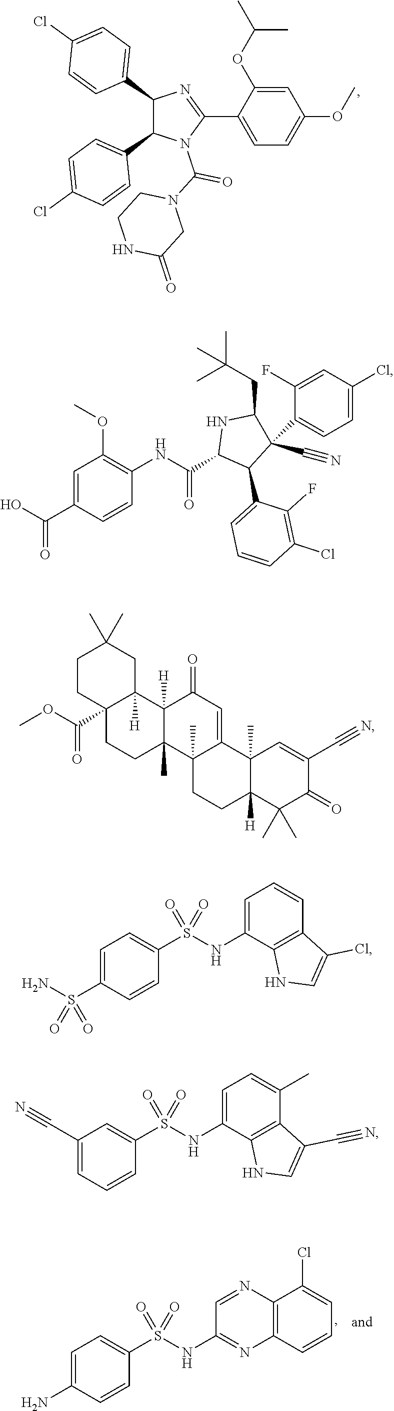

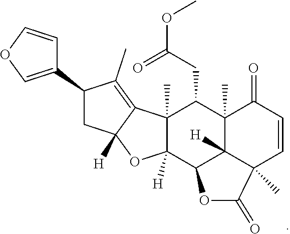

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1 and 2, the steps in some cases may be performed in a different order than the order shown in Schemes 1 and 2. Various modifications to these synthetic reaction schemes may be made and will be suggested to one skilled in the art having referred to the present disclosure. Numberings or R groups in each scheme typically have the same meanings as those defined elsewhere herein unless otherwise indicated.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about−10° C. to about 110° C. over a period of about 1 to about 24 hours; reactions left to run overnight average a period of about 16 hours.

In general, compounds of the disclosure may be prepared by the following reaction schemes:

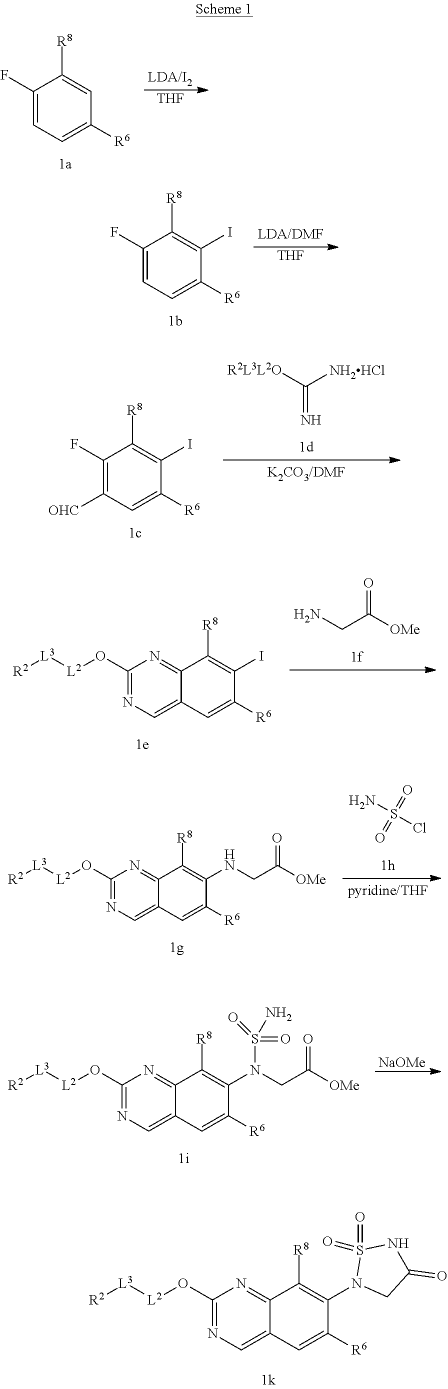

In some embodiments, a compound of Formula 1k may be prepared according to Scheme 1. For example, following iodination of 1a, the resulting aryl iodide derivative (1b) can be treated with LDA and DMF to afford benzaldehyde 1c. Cyclization can proceed upon addition of carbamimidate 1d and a suitable base to give quinazoline 1e. Heteroaryl amine 1g can be formed by coupling aryl iodide 1e with amine 1f. Addition of sulfamoyl chloride (1h) to provide 1i can be followed by cyclization to give a compound of Formula 1k.

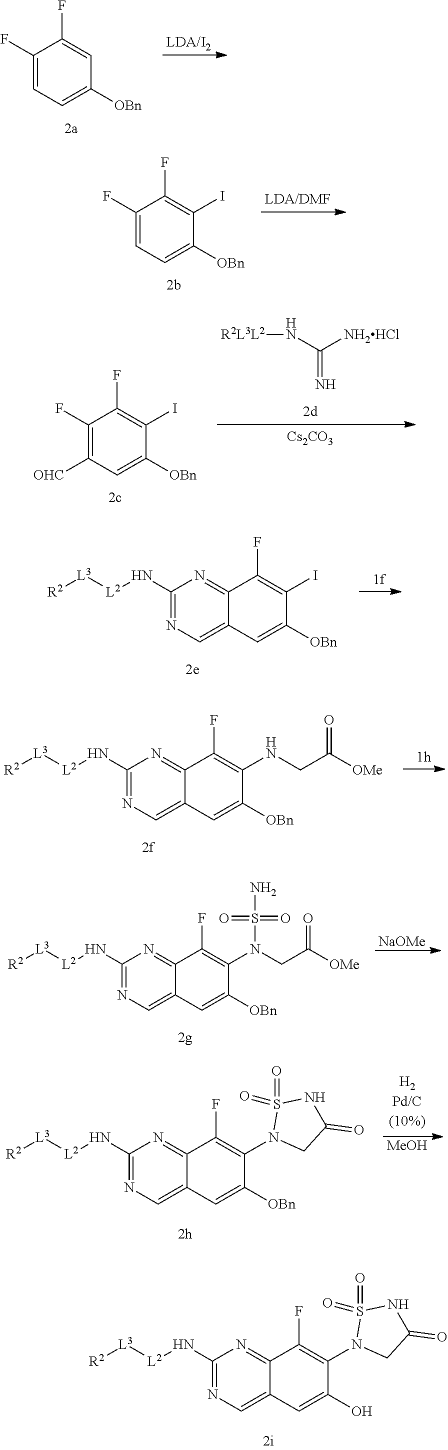

In some embodiments, a compound of Formula 2i may be prepared according to Scheme 2. For example, following iodination of 2a, the resulting aryl iodide derivative (2b) can be treated with LDA and DMF to afford benzaldehyde 2c. Cyclization can proceed upon addition of guanidine 2d and a suitable base to give quinazoline 2e. Heteroaryl amine 2f can be formed by coupling aryl iodide 2e with amine 1f. Addition of sulfamoyl chloride (1h) to provide 2g can be followed by cyclization to give 2h. Deprotection of the benzyl group can provide a compound of Formula 2i.

In some embodiments, a compound of the present disclosure is synthesized according to the general routes outlined in Schemes 1 and 2 or by methods generally known in the art.

It shall be understood that different aspects of the disclosure can be appreciated individually, collectively, or in combination with each other. Various aspects described herein may be applied to any of the particular applications disclosed herein. The compositions of matter, including compounds of any formulae disclosed in the compound section, of the present disclosure may be utilized in the method section, including methods of use and production disclosed herein, or vice versa.

### Methods

Compounds disclosed herein exhibiting anti-PTPN2 activity embody a variety of therapeutic utilities. In an aspect, a PTPN2 inhibitor, such as a compound of Formula (I), can be administered into a subject in need thereof to treat cancer. In some embodiments, a subject PTPN2 inhibitor is systemically and/or transiently (including intermittently) administered to the subject in need thereof to treat one or more types of cancer, including solid tumor and liquid tumor. In another aspect, a subject PTPN2 inhibitor is used to potentiate immunity comprising anti-tumor, anti-cancer activity, anti-viral infection activity, and/or anti-bacterial infection activity in a cell or a subject. In practicing any of the subject methods, a PTPN2 inhibitor disclosed herein can be administered as a single agent. In some embodiments, a PTPN2 inhibitor is administered in combination with another agent as a single or unit dose, or as a separate dose. In some embodiments, the other agent can be a cell, including but not limited to a lymphoid cell (e.g., expressing a CAR and/or TCR). In some embodiment, the other agent can be a second agent including without limitation, chemotherapeutic agent, a radioactive agent, a small molecule agent targeting a tumor marker, an antigen-binding agent specifically binding to a tumor marker, an immune modulator, or any other second agent disclosed herein.

The compounds described herein, or a pharmaceutically acceptable salt or solvate thereof, are PTPN2 inhibitors capable of inhibiting a PTPN2 protein. Compounds, including pharmaceutically acceptable salts or solvates thereof, disclosed herein have a wide range of applications in therapeutics, diagnostics, and other biomedical research. In certain aspects, the present disclosure provides a method of treating cancer in a subject in need thereof, comprising administering to the subject a therapeutically effective amount of a compound described herein, or a pharmaceutically acceptable salt or solvate thereof.

In certain aspects, the present disclosure provides a method of modulating activity of a PTPN2 protein, comprising contacting a PTPN2 protein with an effective amount of a compound described herein, or a pharmaceutically acceptable salt or solvate thereof, thereby modulating the activity of the PTPN2 protein.

In certain aspects, the present disclosure provides a method of inhibiting cell growth, comprising administering an effective amount of a compound described herein, or a pharmaceutically acceptable salt or solvate thereof, to a cell expressing a PTPN2 protein, thereby inhibiting growth of said cells. In some embodiments, the subject method comprises administering an additional agent to said cell.

In certain aspects, the present disclosure provides a method of treating a disease mediated at least in part by a PTPN2 protein in a subject in need thereof, comprising administering to the subject an effective amount of a compound disclosed herein, or a pharmaceutically acceptable salt or solvate thereof. In some embodiments, the disease is cancer, such as a solid tumor or a hematological cancer. In some embodiments, the method further comprises administering an additional agent to the subject, such as a RAS inhibitor, a SHP2 inhibitor, a SOS inhibitor, an EGFR inhibitor, a MEK inhibitor, an ERK inhibitor, a CDK4/6 inhibitor, a BRAF inhibitor, or a combination thereof. In certain aspects, the present disclosure provides a method of treating a PTPN2-mediated cancer in a subject in need thereof, comprising administering to the subject a RAS inhibitor, a SHP2 inhibitor, a SOS inhibitor, an EGFR inhibitor, a MEK inhibitor, an ERK inhibitor, a CDK4/6 inhibitor, or a BRAF inhibitor and an effective amount of a compound disclosed herein, such as a compound of Formula (I), or a pharmaceutically acceptable salt or solvate thereof.

In certain aspects, the present disclosure provides a method of inhibiting activity of a PTPN2 protein comprising contacting the PTPN2 protein with a compound disclosed herein, or a pharmaceutically acceptable salt or solvate thereof. In some embodiments, the compound exhibits an IC₅₀ against the PTPN2 protein of less than 10 μM, such as less than 5 μM, 1 μM, 500 nM, 100 nM, 50 nM, 10 nM, 5 nM, 1 nM, 500 pM, 50 pM, 10 pM or less.

Not wishing to be bound by any particular theory, a subject PTPN2 inhibitor (e.g., a compound described herein) may be effective in one or more of: stimulating and/or prolonging anti-tumor immunity (e.g., destabilizing Tregs, augmenting CD4+ and CD8+ T cell function, increasing the number of central memory T cells or half-life of such cells), inhibiting proliferation of cancer cells, inhibiting invasion or metastasis of cancer cells, killing cancer cells, increasing the sensitivity of cancer cells to treatment with a second antitumor agent, and reducing severity or incidence of symptoms associated with the presence of cancer cells. In some embodiments, said method comprises administering to the cancer cells a therapeutically effective amount of a PTPN2 inhibitor in vivo. In some embodiments, the administration first takes place ex vivo to a population of effector cells, followed by infusing the PTPN2 inhibitor-treated effector cells into the subject as further detailed below.

In some embodiments, the small molecule PTPN2 inhibitor may not affect editing of (i) a gene encoding PTPN2 or (ii) an additional gene operatively linked to PTPN2 (e.g., transcription factor, intron sequence, start codon, etc.). As such, the gene and/or the additional gene may remain the same upon treatment of a cell with a small molecule PTPN2 inhibitor, such as a compound of Formula (I). In some embodiments, the small molecule PTPN2 inhibitor may be configured to bind at least a portion of PTPN2. The small molecule may exhibit binding specificity to PTPN2 in comparison to one or more other protein tyrosine phosphatases selected from the group consisting of: PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV, PTPRZ, PTPN1, PTPN2, PTPN3, PTPN4, PTPN5, PTPN6, PTPN7, PTPN9, PTPN11, PTPN12, PTPN13, PTPN14, PTPN18, PTPN20, PTPN21, PTPN23, DUSP1, DUSP2, DUSP4, DUSP5, DUSP6, DUSP7, DUSP8, DUSP9, DUSP10, DUSP16, MK-STYX, DUSP3, DUSP11, DUSP12, DUSP13Aa, DUSP13Ba, DUSP14, DUSP15, DUSP18, DUSP19, DUSP21, DUSP22, DUSP23, DUSP24, DUSP25, DUSP26, DUSP27b, EPM2A, RNGTT, STYX, SSH1, SSH2, SSH3, PTP4A1, PTP4A2, PTP4A3, CDCl4A, CDCl4B, CDKN3, PTP9Q22, PTEN, TPIP, TPTE, TNS, TENC1, MTM1, MTMR1, MTMR2, MTMR3, MTMR4, MTMR5, MTMR6, MTMR7, MTMR8, MTMR9, MTMR10, MTMR11, MTMR¹², MTMR13, MTMR14, MTMR15, ACP1, CDC25A, CDC25B, CDC25C, EYA1, EYA1, EYA1, and EYA1. In some embodiments, a subject compound, such as a compound of Formula (I), specifically binds to PTPN2 relative to PTP1B. In some embodiments, a subject compound selectively inhibits PTPN2 relative to PTP1B. In some cases, a subject compound may exhibit a half maximal inhibitory concentration (i.e., IC₅₀) of less than or equal to about 10 micromolar (uM), 5 μM, 1 μM, 950 nanomolar (nM), 900 nM, 850 nM, 800 nM, 750 nM, 700 nM, 650 nM, 600 nM, 550 nM, 500 nM, 450 nM, 400 nM, 350 nM, 300 nM, 250 nM, 200 nM, 150 nM, 100 nM, 50 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, or less for PTPN2. The small molecule PTPN2 inhibitor may exhibit an IC₅₀ for PTPN2 that is at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 11-fold, 12-fold, 13-fold, 14-fold, 15-fold, 16-fold, 17-fold, 18-fold, 19-fold, 20-fold, 30-fold, 40-fold, 50-fold, 60-fold, 70-fold, 80-fold, 90-fold, 100-fold, or more potent than that of one or more other protein tyrosine phosphatases (e.g., IC₅₀ concentration is a lower number for PTPN2 than another PTP). In different embodiments, the small molecule PTPN2 inhibitor may be configured to bind at least a portion of one or more substrates of PTPN2 selected from the group consisting of: INSR, EGFR, CSF1R, PDGFR, JAK1, JAK2, JAK3, Src family kinases, STAT1, STAT3, STAT6, FYN, LCK, variations thereof, and combinations thereof.

In some embodiments, a compound of the present disclosure, such as a compound of Formula (I), may be conjugated to a degradation tag (i.e., degradation enhancer). A degradation tag may be configured to bind a degradation moiety having a capacity to degrade at least a portion of a target moiety that is bound by the degradation tag. For example, the target moiety is PTPN2 or the substrate of PTPN2. A degradation tag may be a biological or chemical compound, such as a simple or complex organic or inorganic molecule, peptide, peptido mimetic, protein (e.g., antibody), liposome, or a polynucleotide (e.g., small interfering RNA, short hairpin RNA, microRNA, antisense, aptamer, ribozyme, triple helix). Alternatively, a degradation tag may be synthetic. In some cases, any one of the methods described herein may utilize a small molecule degradation tag, and non-limiting examples of such small molecule degradation tag may include, but are not limited to, pomalidomide, thalidomide, lenalidomide, VHL-1, adamantane, 1-((4,4,5,5,5-pentafluoropentyl) sulfinyl) nonane, nutlin-3a, RG7112, RG7338, AMG 232, AA-115, bestatin, MV-1, LCL161, and/or analogs thereof. In some cases, the degradation tag can (i) bind to a degradation moiety such as a ubiquitin ligase (e.g., an E3 ligase such as a cereblon E3 ligase, a VHL E3 ligase, a MDM2 ligase, a TRIM21 ligase, a TRIM24 ligase, and/or a IAP ligase) and/or (ii) serve as a hydrophobic group that leads to protein misfolding of the target moiety, e.g., PTPN2. Misfolding of the target moiety may disrupt activity of the target moiety and/or increase the likelihood of degradation of the target moiety by, e.g., a degradation moiety. In some cases, a small molecule PTPN2 inhibitor, such as a compound of Formula (I), may be conjugated to the degradation tag via a linker. Examples of such linker may include, but are not limited to, acyclic or cyclic saturated or unsaturated carbon, ethylene glycol, amide, amino, ether, urea, carbamate, aromatic, heteroaromatic, heterocyclic, and/or carbonyl containing groups with different lengths. Exemplary molecules comprising such degradation tag and method of use thereof are provided in U.S. Patent Publication No. 2019/0336503, which is incorporated herein by reference in its entirety.

In some embodiments, a method of the disclosure provides an effective amount of a PTPN2 inhibitor, such as a compound of Formula (I). An effective dose refers to an amount sufficient to affect the intended application, including treatment of cancer and stimulating or prolonging anti-tumor immunity. Also contemplated in the subject methods is the use of a sub-therapeutic amount of a PTPN2 inhibitor for treating an intended disease condition.

The amount of the PTPN2 inhibitor, such as a compound of Formula (I), administered may vary depending upon the intended application (in vitro, ex vivo, or in vivo), or the subject and cancer condition being treated, e.g., the weight and age of the subject, the severity of the cancer, the manner of administration and the like. In some cases, a PTPN2 inhibitor may be administered (e.g., systemically administered) to a subject at a dose of at least about 0.1 milligrams per kilogram (mg/kg), 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, or more. In some cases, a PTPN2 inhibitor may be administered (e.g., systemically administered) to a subject at a dose of at most about 50 mg/kg, 45 mg/kg, 40 mg/kg, 35 mg/kg, 30 mg/kg, 25 mg/kg, 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, 0.1 mg/kg, or less.

In some cases, upon administration (e.g., systemic administration), a mean plasma concentration of the PTPN2 inhibitor, such as a compound of Formula (I), in the subject may be at least about 0.1 microgram per milliliter (μg/ml), 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 25 μg/ml, 30 μg/ml, 35 μg/ml, 40 μg/ml, 45 μg/ml, 50 μg/ml, or more. In some cases, upon administration (e.g., systemic administration), a mean plasma concentration of the PTPN2 inhibitor in the subject may be at most about 50 μg/ml, 45 μg/ml, 40 μg/ml, 35 μg/ml, 30 μg/ml, 25 μg/ml, 20 μg/ml, 19 μg/ml, 18 μg/ml, 17 μg/ml, 16 μg/ml, 15 μg/ml, 14 μg/ml, 13 μg/ml, 12 μg/ml, 11 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, 0.1 μg/ml, or less.

In some embodiments, a PTPN2 inhibitor, such as a compound of Formula (I), may be used in combination with another known agent (a second agent) or therapy. Examples of such second agent may be selected from the group consisting of a chemotherapeutic agent, a radioactive agent, a small molecule agent targeting a tumor marker, an antigen-binding agent specifically binding to a tumor marker, and an immune modulator. An immune modulator may be selected from the group consisting of immunostimulatory agents, checkpoint immune blockade agents, and combinations thereof. In some embodiments, the second agent may be a checkpoint inhibitor. In some examples, the second agent may be an inhibitor of PD1, PD-L1, LAG3, CTLA4, CD160, BTLA, LAIR1, TIM3, 2B4, CD93, OX40, Siglec-15, and TIGIT. A PTPN2 inhibitor can be administered as part of a therapeutic regimen that comprises administering one or more second agents (e.g., 1, 2, 3, 4, 5, or more second agents), either simultaneously or sequentially with the PTPN2 inhibitor. When administered sequentially, the PTPN2 inhibitor may be administered before, concurrent with, or after the one or more second agents. When administered simultaneously, the PTPN2 inhibitor and the one or more second agents may be administered by the same route (e.g. injections to the same location; tablets taken orally at the same time), by a different route (e.g. a tablet taken orally while receiving an intravenous infusion), or as part of the same combination (e.g. a solution comprising the PTPN2 inhibitor and one or more second agents). In some examples, a PTPN2 inhibitor can be used in combination with a cell therapy, including a TFP- or CAR-expressing cell (e.g., a TFP- or CAR-expressing stem cell or lymphoid cell) described herein. In other examples, a PTPN2 inhibitor can be used in combination with a non-cell based therapy, such as surgery, chemotherapy, targeted therapy (e.g., using large or small drug molecules targeting a tumor antigen other than PTPN2), radiation, and the like.

In some embodiments, a PTPN2 inhibitor described herein, such as a compound of Formula (I), is administered to a subject in combination with an indoleamine 2,3-dioxygenase (IDO) inhibitor. IDO is an enzyme that catalyzes the degradation of the amino acid L-tryptophan to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by any particular theory, it has been reported that a decrease in L-tryptophan (e.g., catalyzed by IDO) results in an immunosuppressive milieu by inducing T-cell anergy and apoptosis. It is thought that IDO inhibitor can enhance the efficacy of a CAR-expressing cell by decreasing the suppression or death of a CAR-expressing immune cell. While the clinical trial involving the combination of pembrolizumab (an anti-PD1 antibody) and epacadostat (an IDO inhibitor) did not reach the desired end point, a PTPN2 inhibitor is expected to potentiate the therapeutic effect of IDO inhibitor. Without being bound by a particular theory, PTPN2 inhibitors are expected to destabilize the function of the already activated regulatory T-cells while the IDO inhibitors prevent the activation of new regulatory T-cells. Exemplary inhibitors of IDO that can be used in combination include but are not limited to 1-methyl-tryptophan, indoximod (NewLink Genetics)(see, e.g., Clinical Trial Identifier Nos. NCT01191216; NCT01792050), and INCB024360 (Incyte Corp.)(see, e.g., Clinical Trial Identifier Nos. NCT01604889; NCT01685255).

Additional agents that can be used in combination with a PTPN2 inhibitor, such as a compound of Formula (I), include the various categories and examples of agents listed in Table 1 below.

TABLE 1

| | |
|---|---|
| Alkylating agents | Examples include, but are not limted to, altretamine (Hexalen ® or hexamethylmelamine or |
| | HMM), bendamustine, busulfan (Busulfex ® or Myleran ®), carmustine (BiCNU ® or |
| | BCNU ®), chlorambucil, cyclophosphamide (Cytoxan ® or Neosar ®), dacarbazine (DTIC- |
| | Dome ®), fotemustine, ifosfamide (Ifex ®), improsulfan, lomustine (CCNU ® or CeeNU ®), |
| | mechlorethamine or mustine (Mustardgen ®), melphalan (Alkeran ®), nimustine, |
| | piposulfan, ranimustine, semustine, streptozocin (Zanosar ®), temozolomide (Temodar ®), |
| | thiotepa (Thioplex ®), trofosfamide (Ixoten ®), and uramustine or uracil mustard |
| Anthracyclines | Examples include, but are not limited to, aclarubicin, amrubicin, daunorubicin |
| | (Daunomycin ®), daunorubicin (liposomal), doxorubicin (Adriamycin ®), doxorubicin |
| | (liposomal), epirubicin, esorubicin, idarubicin, mitoxantrone, pirarubicin, and valrubicin |
| Anti-angiogenic | Examples include, but are not limited to, aflibercept, axitinib (Inlyta ®), bevacizumab |
| | (Avastin ®), cabozantinib (Cometriq ®), everolimus (Afinitor ® or Zortress ®), lenalidomide |
| | (Revlimid ®), pazopanib (Votrient ®), ponatinib, ramucirumab (Cyramza ®), ranibizumab, |
| | regorafenib (Stivarga ®), sorafenib (Nexavar ®), sunitinib (Sutent ®), thalidomide |
| | (Synovir ® or Thalomid ®) lapatinib, and vandetanib (Caprelsa ®) |
| Antifolates | Examples include, but are not limited to, aminopterin, edatrexate, folic acid, GW1843, |
| | lometrexol, LY309887, methotrexate, nolatrexed, OSI-7904(L), pemetrexed (Alimta ®), |
| | pralatrexate, raltitrexed, trimetrexate, and ZD9331 |
| BCL-2 inhibitors | Examples include, but are not limited to, ABT-199, ABT-263, ABT-737, BH3 mimetics, |
| | gossypol, obatoclax (GX15-070), oblimersen (Genasense ®; G3139; Augmerosen ®), and |
| | venetoclax |
| Bcr-Abl inhibitors | Examples include, but are not limited to, bafetinib (INNO-406), bosutinib (SKI-606), |
| | dasatinib (BMS-354825), imatinib (STI-571), nilotinib (AMN-107), and ponatinib (AP- |
| | 24534 or Iclusig ®) |
| Biologics | Examples include, but are not limited to, BCG (TheraCys ®), cytokines (such as INF-alfa, |
| | Aldesleukin, erythropoietin, GM-CSF, G-CSF), gene therapy (such as CT109 or |
| | Kymriah ®), MAbs (such as rituximab, alemtuzumab, ipilimumab, bevacizumab, |
| | obinutuzumab, brentuximab), oncolytic viruses (such as T-VEC or Imlygic ®, H101), and |
| | treatment vaccines (such as sipuleucel-T or Provenge ®) |
| CDK inhibitors | Examples include, but are not limited to, A-674563, abemaciclib (LY2835219), AT-7519, |
| | AZD5438, BMS-265246, BS-181, CYC202 (roscovitine; seliciclib), dinaciclib (SCH- |
| | 727965), flavopiridol (alvocidib), indirubin, JNJ-7706621, K03861, kenpaullone, |
| | LDC000067, LDC4297 (LDC044297), LY2857785, MK-8776 (SCH900776), ML167, |
| | MSC2530818, NU6027, olomoucine, ON123300, P276-00, palbociclib (PD-0332991), |
| | PHA-767491, PHA-793887, PHA-848125 (milciclib), purvalanol A, purvalanol B, R547, |
| | ribociclib (LEE011), RO-3306, senexin A, SNS-032 (BMS-387032), SU9516, TG003, |
| | THZ1, UCN-01 (7-hydroxystaurosporine; KRX-0601), wogonin, and XL413 |
| COX-2 inhibitors | Examples include, but are not limited to, celecoxib (Celebrex ®), etoricoxib, lumiracoxib |
| | (Prexige ®), parecoxib (Dynastat ®), rofecoxib, and valdecoxib (Bextra ®) |
| CTLA-4 inhibitors | Examples include, but are not limited to, ipilimumab (MDX-010) and tremelimumab |
| | (10D1) |
| DNA | Examples include, but are not limited to, 1-hydrazinophthalazine, 5,6-dihydro-5- |
| methyltransferase | azacytidine, 5-aza-2′-deoxycytidine, 5-aza-C (5-azacytidine; azacytidine), 5-fluoro-2′- |
| inhibitors | deoxycytidine, arabinosyl-5-azacytidine, decitabine, disulfiram, doxorubicin, EGCG, |
| | EGX30P, hydralazine, MG98, nanaomycin A, nanaomycin C, procainamide, procaine, |
| | psammaplin A, RG108, SGI-1027, sinefungin, thioguanine, and zebularine |
| ERK inhibitors | Examples include, but are not limited to, AG1478, AG99, andrographolide, apigenin, BAY |
| | 43-9006, CAY10561, DEL-22379, ERK inhibitor III, ERK5-IN-1, FR 180204, GDC0994, |
| | GDC-0994, GW5074, hypericin, ISIS 5132, KO947, MK-8353 (SCH900353), ML-9, |
| | PD169316, PD173074, pluripotin, purvalanol, pyrazolylpyrrole ERK inhibitor, SB203580, |
| | SC-1, SCH772984, SL327, SP600125, SU4984, ulixertinib (BVD-523, VRT752271), VX- |
| | 11e (ERK-11e; TCS ERK 11e), and XMD 8-92 |
| Farnesyltransferase | Examples include, but are not limited to, BMS-214662, CP-609754, DK8G557, FTI-277, |
| inhibitors | L744832, L778123, lonafarnib (Sarasar ®; SCH66336), manumycin A, and R115777 |
| | (Zarnestra ®; tipifarnib |
| FLT3 inhibitors | Examples include, but are not limited to, crenolanib besylate (CP-868596-26), gilteritinib |
| | (ASP2215), lestaurtinib (CEP-701), midostaurin (Rydapt ®, PKC412), pexidartinib |
| | (PLX3397), ponatinib (AP24534), quizartinib (AC220), SKLB1028, sorafenib (Nexavar ®), |
| | sunitinib (Sutent ®, SU11248), and XL999 |
| HDAC inhibitors | Examples include, but are not limited to, 4SC-202, Abexinostat (PCI-24781), apicidin, AR- |
| | 42, ATRA, Belinostat (PXD101), BG-45, BRD73954, CAY10603, CG200745, Chidamide, |
| | CHR-2845, CHR-3996, Citarinostat (ACY-241), CUDC-101, CUDC-907, curcumin, |
| | dacinostat (LAQ824), droxinostat, Entinostat (MS-275), FR901228, Givinostat (ITF2357), |
| | HBI-8000, HPOB, ITSA-1, Kevetrin, LAQ824, largazole, LMK-235, M344, MC-1568, m- |
| | carboxycinnamic acid bishydroxamide, ME-344, Mocetinostat (MGCD0103), nexturastat |
| | A, oxamflatin, Panobinostat (LBH589), pracinostat (SB939), pyroxamide, Quisinostat |
| | (JNJ-26481585), Resminostat (4SC-201), RG2833 (RGFP109), RGFP966, ricolinostat |
| | (ACY-1215), Romidepsin (FK228; Depsipeptide), Romidepsin (Istodax), santacruzamate A |
| | (CAY10683), SB939, SBHA, scriptaid, sulforaphane, tacedinaline (CI994) PCI-34051, |
| | tasquinimod, TMP-195, TMP-269, trapoxin A, trichostatin A, tubacin, tubastatin A, |
| | Valproic acid (as Mg valproate), and Vorinostat (SAHA |
| Hedgehog | Examples include, but are not limited to, AY 9944, BMS-833923 (XL139), GANT58, |
| signaling inhibitors | GANT61, HPI-4, JK184, LEQ 506, PF-04449913, SANT-1, SANT-2, sonidegib (LDE- |
| | 225; Odomzo ®), TAK-441, and vismodegib (IPI-926; Erivedge ®) |
| HIF inhibitors | Examples include, but are not limited to, 2-methoxyestradiol, bortezomib, camptothecin, |
| | echinomycin, ENMD-1198, miltefosine, perifosine, romidepsin, and temsirolimus |
| Hormone therapies | Examples include, but are not limited to, 4(5)-imidazoles, 4-hydroxytamoxifen, |
| | aminoglutethimide, anastrozole (Arimidex ®), bicalutamide megestrol acetate, |
| | diethylstilbestrol, estrace, exemestane (Aromasin ®), fluoxymesterone, flutamide, |
| | fulvestrant (Faslodex ®), goserelin (Zoladex ®), keoxifene, letrozole (Femara ®), |
| | leuprorelin, LY 117018, medroxyprogesterone acetate, nilutamide, octreotide, onapristone, |
| | polyestradiol phosphate, raloxifene, tamoxifen, toremifene, toremifene (Fareston ®), and |
| | trioxifene |
| MEK inhibitors | Examples include, but are not limited to, AR-119/RDEA119 (BAY 869766), arctigenin, |
| | ARRY-438162, AS-701173, AS-701255, AS703026, AZD6244 (ARRY-142886), |
| | AZD8330 (ARRY-704), binimetinib (MEK162), CI-1040 (PD184352), E6201, GDC-0623, |
| | GDC-0973 (XL518; cobimetinib), hypothemycin, PD0325901, PD0325901, PD181461, |
| | PD98059, pimasertib, refametinib, RO09-2210, RO4927350, RO4987655, RO5068760, |
| | RO5126766, selumetinib, TAK-733, trametinib (GSK1120212), U0126, and WX-554 |
| Mitotic inhibitors | Examples include, but are not limited to, colchicine, glaziovianin A, griseofulvin, |
| | podophyllotoxin, taxanes, vinblastine, vincristine, vindesine, and vinorelbine |
| mTOR inhibitors - | Examples include, but are not limited to, ABT578, AZD2014, AZD8055, BEZ235, CC- |
| immune enhancing | 223, everolimus (RAD001), GSK2126458, INK128 (MLN-0128), Ku-0063794, |
| amount which is | LY294002, NVP-BEZ235, OSI-027, PI-103, PP242, rapamycin (Sirolimus ®), |
| typically below the | ridaforolimus (Deforolimus ®; AP-23573), temsirolimus (CCI-779), and XL765 |
| therapeutic dose | |
| Multi-kinase | Examples include, but are not limited to, AT9283, dasatinib, lestaurtinib, midostaurin |
| inhibitors | (Rydapt ®), motesanib, neratinib (HKI-272), nilotinib, pazopanib, regorafenib, sorafenib, |
| | sunitinib, vandetanib, and XL184 |
| Nitrogen mustards | Examples include, but are not limited to, bendamustine, chlorambucil, chlornaphazine, |
| | cyclophosphamide, estramustine, ifosfamide, mechlorethamine, mechlorethamine oxide |
| | hydrochloride, melphalan, mustine, novembichin, phenesterine, prednimustine, |
| | trofosfamide, uracil mustard, and uramustine |
| DNA synthesis | Examples include, but are not limited to, 3,4-dihydroxybenzylamine, 5-fluorodeoxyuridine, |
| inhibitors | cladribine, cytarabine, daunorubicin, etoposide, flutarabine, hydroxyurea, idarubicin, |
| | methotrexate, mitoxantrone, and pralatrexate |
| PD-1 inhibitors | Examples include, but are not limited to, AMP-224, AMP-514, atezolizumab (RG7446; |
| | MPDL3280A), BMS-936559, durvalumab, MAB005, MDX-1106, MEDI4736, |
| | MSB0010718C (A09-246-2), nivolumab (BMS936558), pembrolizumab |
| | (Lambrolizumab ®; MK-3475; KEYTRUDA ®), and pidilizumab (CT-011) |
| | Anti PD-1 antibodies: Examples include, but are not limited to, ABCAM ® (AB137132), |
| | AFFYMETRIX EBIOSCIENCE (J105; J116; M1H4), Amplimmune (AMP 514), avelumab |
| | (Bavencio ®), and BIOLEGEND ® (EH12.2H7; RMPI-14) |
| RAF inhibitors | Examples include, but are not limited to, AZ 628, CCT196969, CEP-32496, dabrafenib |
| | (GSK2118436), encorafenib (LGX818), GDC-0879, GW5074, LY3009120, MLN2480, |
| | NVP-BHG712, PLX-4720, PLX-7904, RAF265, RO5126766 (CH5126766), SB590885, |
| | sorafenib tosylate, TAK-632, vemurafenib (PLX4032; RG7204), and ZM 336372 |
| RAS inhibitors | Examples include, but are not limited to, deltarasin, lonafarnib, reolysin, salirasib, siG12D |
| | LODER, SML-8-73-1, and tipifarnib |
| Others | Examples include, but are not limited to, 5-aminolevulinic acid, afatinib, alectinib, |
| | altretamine, anti-CD3 and/or anti-CD33 antibodies (e.g. visilizumab, gemtuzumab, |
| | AMG330), antiproliferative antibodies, aprepitant, arsenic trioxide, benzodopa, biological |
| | response modifiers, bisphosphonates (such as etridonic, clodronic, tiludronic, pamidronic, |
| | alendronic, ibandronic, risedronic, zoledronic acid), bleomycin, blinatumomab (AMG103; |
| | Blincyto ®), brigatinib (Alunbrig ®), buserelin, cabazitaxel, carboquone, ceritinib, |
| | crizotinib, dactinomycin, degarelix, denosumab, dexamethasone, difluoromethylornithine |
| | (DFMO), dronabinol, enasidenib (AG-221), epothilone A or B, eribulin, erlotinib, gefitinib, |
| | gonadorelin agonists, granisetron, heparanase inhibitors, histone deacetylase inhibitors, |
| | histrelin, ibrutinib, inotuzumab ozogamicin (Besponsa ®), ixabepilone, lenvatinib, |
| | leuprolide, levoleucovorin, matrix metalloproteinase inhibitors (such as marimastat (BB- |
| | 2516), prinomastat (AG3340), BMS-279251, BAY 12-9566, TAA211, MMI270B, |
| | AAJ996), methionine aminopeptidase inhibitors, meturedopa, mitomycin, mitotane, |
| | nabilone, necitumumab, niraparib (Zejula ®), olaparib (AZD-2281; Lynparza ®), |
| | osimertinib (Tagrisso ®), palonosetron, panitumumab, pertuzumab, porfimer sodium, |
| | revlimid, thalidomide, tipiracil, trabectedin, trastuzumab, triethylenemelamine, |
| | triethylenephosphoramide, triethylenethiophosphoramide, trimethylolomelamine, and |
| | uredopa |

In embodiments, a compound described herein, such as a compound of Formula (I), may be administered alone or in combination or in conjunction with another therapy or another agent. By “combination” it is meant to include (a) formulating a subject composition containing a subject compound, such as a compound of Formula (I), together with another agent, and (b) using the subject composition separate from the other agent as an overall treatment regimen. By “conjunction” it is meant that the other therapy or agent is administered either simultaneously, concurrently or sequentially with a subject composition comprising a compound disclosed herein, with no specific time limits, wherein such conjunctive administration provides a therapeutic effect.

In some embodiment, a subject treatment method (e.g., a method comprising a compound described herein) is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, immunomodulatory agents, and combinations thereof.

In an aspect, compositions provided herein can be administered in combination with radiotherapy, such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation, it may be uniform or not uniform. For example, when radiation may not be uniform, narrower regions of a body such as the neck may receive a higher dose than broader regions such as the hips.

In some other embodiments, any of the compounds herein that is capable of modulating a PTPN2 protein may be administered in combination or in conjunction with one or more pharmacologically active agents including but not limited to: (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R⁴²⁸, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor (e.g., 6-(4-amino-4-methylpiperidin-1-yl)—3-(2,3-dichlorophenyl) pyrazin-2-amine), as well as SHIP1 inhibitors; (27) an inhibitor of SRC(e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib); (31) a ROS1 inhibitor (e.g., entrectinib); (32) an inhibitor of FLT3, HDAC, VEGFR, PDGFR, LCK, Bcr-Abl or AKT; (33) an inhibitor of SHP pathway; (34) an inhibitor of KrasG12C mutant (e.g., including but not limited to AMG510, MRTX849, and any covalent inhibitors binding to the cysteine residue 12 of Kras, the structures of these compounds are publicly known)(e.g., an inhibitor of Ras G12C as described in US20180334454, US20190144444, US20150239900, U.S. Pat. No. 10,246,424, US20180086753, WO2018143315, WO2018206539, WO20191107519, WO2019141250, WO2019150305, U.S. Pat. No. 9,862,701, US20170197945, US20180086753, U.S. Pat. No. 10,144,724, US20190055211, US20190092767, US20180127396, US20180273523, U.S. Pat. No. 10,280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety); (35) an SHC inhibitor (e.g., PP2, AID371185); (36) a GAB inhibitor (e.g., GAB-0001), (37) a GRB inhibitor; (38) a PI-3 kinase inhibitor (e.g., Idelalisib, Copanlisib, Duvelisib, Alpelisib, Taselisib, Perifosine, Buparlisib, Umbralisib, NVP-BEZ235-AN); (39) a MARPK inhibitor; (40) CDK4/6 (e.g., palbociclib, ribociclib, abemaciclib); (41) a MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197); (42) an inhibitor of SHP pathway including SHP2 inhibitor (e.g., 6-(4-amino-4-methylpiperidin-1-yl)—3-(2,3-dichlorophenyl) pyrazin-2-amine, RMC-4630, ERAS-601,

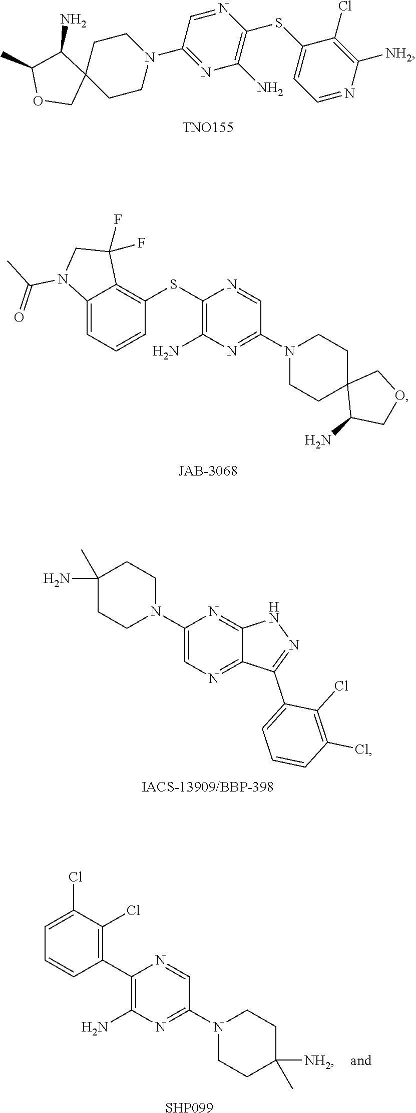 [SMILES: "TNO155" ; C[C@@H]1OCC2(CCN(c3cnc(Sc4ccnc(N)c4Cl)c(N)n3)CC2)[C@@H]1N ; CC(=O)N1CC(F)(F)c2c(Sc3ncc(N4CCC5(CC4)COC[C@H]5N)nc3N)cccc21 ; CC1(N)CCN(c2cnc3c(-c4cccc(Cl)c4Cl)n[nH]c3n2)CC1 ; "SHP099" ; CC1(N)CCN(c2cnc(-c3cccc(Cl)c3Cl)c(N)n2)CC1]

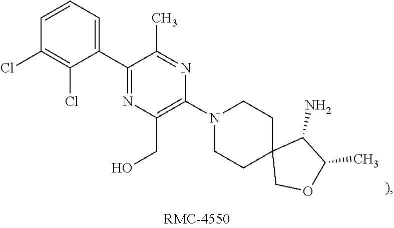 [SMILES: Cc1nc(N2CCC3(CC2)CO[C@@H](C)[C@H]3N)c(CO)nc1-c1cccc(Cl)c1Cl]

as well as SHP1 inhibitors; or (43) an inhibitor of a Kras mutant (e.g., Kras G12D, including a compound described in WO2021041671, WO2021107160, WO2021091967, WO2021142252, WO2021150613, WO2021211864, WO2021118877, WO2021081212, WO2021108683; KRas G12C, KRas G12D, KRas G12S, KRas G12V, KRas G13D, KRas G13C, or KRas G13V). In some embodiments, any of the compounds herein that is capable of inhibiting a PTPN2 protein may be administered in combination or in conjunction with one or more checkpoint immune blockade agents (e.g., anti-PD-1 and/or anti-PD-L1 antibody, anti-CLTA-4 antibody). In embodiments, a compound described herein may be administered in combination or conjunction with a SOS(e.g., SOS1) inhibitor, including a compound described in WO2021173524, WO2021203768, WO2020180770, WO2020180768, WO2021092115, WO2018172250, WO2019201848, WO2018115380, WO2019122129, or WO2021127429; all of which are herein incorporated by reference for any purpose. In some embodiments, the SOS inhibitor is selected from RMC-5845, BI-1701963,

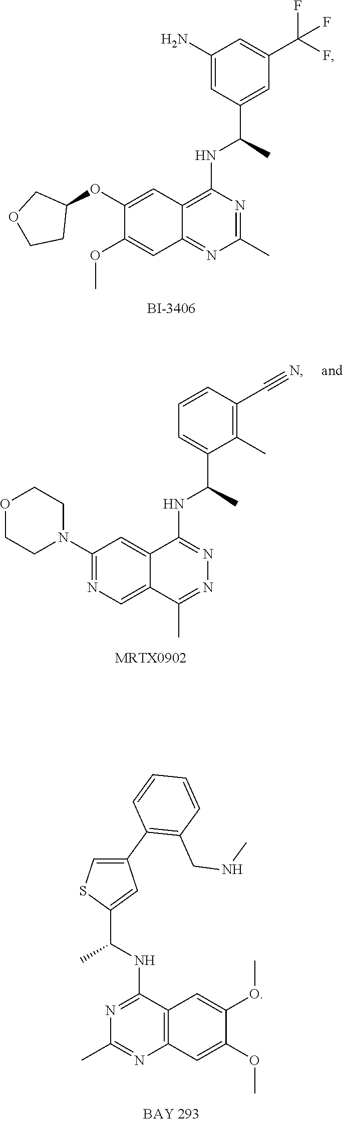

In an aspect, the present disclosure provides a method of potentiating immunity of a subject in need thereof, comprising administering (e.g., systemically administering) a PTPN2 inhibitor, such as a compound of Formula (I), to the subject, thereby potentiating immunity of the subject. In another aspect, the present disclosure provides a method of potentiating immunity of a subject in need thereof, comprising (e.g., transiently) downregulating expression or activity of PTPN2 in vivo in a cell of the subject, thereby potentiating immunity of the subject. In another aspect, the present disclosure provides a method of potentiating immunity of a subject in need thereof, comprising (a) selecting the subject, wherein a cell of the subject exhibits expression or activity of PTPN2; and (b) downregulating the expression or activity of PTPN2 in a cell of the subject, thereby potentiating immunity of the subject. In another aspect, the present disclosure provides a method of potentiating immunity of a subject in need thereof, comprising (a) administering a lymphoid cell to the subject, wherein the lymphoid cell comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen; and (b) separately administering a PTPN2 inhibitor, such as a compound of Formula (I), to the subject, thereby potentiating immunity of the subject. In another aspect, the present disclosure provides a method of potentiating immunity of a cell, comprising (a) contacting the cell with a PTPN2 inhibitor; and (b) introducing to the cell (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell, wherein (a) is performed prior to or concurrent with (b), thereby potentiating immunity of the cell.

In another aspect, the present disclosure provides a method of increasing efficacy or reducing a side effect of a cell therapy for a subject in need thereof, comprising (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and (b) administering a PTNP2 inhibitor, such as a compound of Formula (I), to said subject prior to, concurrent with, or subsequent to (a). In another aspect, the present disclosure provides a method of increasing efficacy or reducing a side effect of a cell therapy for a subject in need thereof, comprising (a) administering to the subject a sub-therapeutic amount of a cell comprising a chimeric antigen receptor (CAR) sequence encoding a CAR, and (b) administering a PTNP2 inhibitor to said subject prior to, concurrent with, or subsequent to (a).

In practicing any of the methods disclosed herein, a cell or a plurality of such cell may be administered (e.g., systemically administered) to the subject. In some cases, the cell may be a lymphoid cell that optically comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the cell may be administered (e.g., systemically administered) to the subject sequentially (e.g., prior to or subsequent to) or concurrent with administering (e.g., systemically administering) a PTPN2 inhibitor to the subject. The cell may have been contacted previously with a PTPN2 inhibitor. Alternatively, the cell may not or need not be contacted with a PTPN2 inhibitor prior to the administration of the cell to the subject.

In some embodiments, (i) the chimeric T-cell receptor sequence and/or (ii) the CAR sequence may be introduced to the cell directly (e.g., via a solution comprising (i) the chimeric T-cell receptor sequence and/or (ii) the CAR sequence), by chemical means (e.g., via one or more carriers such as liposomes for delivery of one or more nucleic acid sequences comprising (i) the chimeric T-cell receptor sequence and/or (ii) the CAR sequence), and/or viral means (e.g., when delivering one or more nucleic acid sequences comprising (i) the chimeric T-cell receptor sequence and/or (ii) the CAR sequence). For the viral means, the one or more nucleic acid sequence may in introduced in a chromosome of the cell, such as a nuclear chromosome and/or a mitochondrial chromosome. In other embodiments, the one or more nucleic acid sequence may not or need not be introduced in the chromosome of the cell, and as such be introduced to the cell as an epichromosomal molecule (e.g., a linear or circular nucleic acid molecule). In some embodiments, the cell may be a lymphoid cell.

Subsequent to the introduction, (i) the chimeric T-cell receptor sequence and/or (ii) the CAR sequence may persist in the cell for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, or more, or any time in between. Subsequent to the introduction, (i) the chimeric T-cell receptor sequence and/or (ii) the CAR sequence may persist in the cell for at most 5 years, 4 years, 3 years, 24 months, 23 months, 22 months, 21 months, 20 months, 19 months, 18 months, 17 months, 16 months, 15 months, 14 months, 13 months, 12 months, 11 months, 10 months, 9 months, 8 months, 7 months, 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less, or any time in between.

In some embodiments, introducing to the cell (i) the chimeric T-cell receptor sequence and/or (ii) the CAR sequence may be performed sequentially (e.g., prior to or subsequent to) or concurrent with contacting the cell with a PTPN2 inhibitor. When introduced sequentially, introducing (i) the chimeric T-cell receptor sequence and/or (ii) the CAR sequence and contacting with the PTPN2 inhibitor may be performed by the same route (e.g. injections to the same location; tablets taken orally at the same time), or by a different route (e.g. a tablet taken orally while receiving an intravenous infusion). When introduced concurrently, for example, a first composition comprising (i) the chimeric T-cell receptor sequence and/or (ii) the CAR sequence and a second composition comprising the PTPN2 inhibitor may be part of the same composition (e.g., the same condition media or a therapeutic regimen).

Contacting the cell with the PTPN2 inhibitor, whether systemically and/or transiently, as described in the present disclosure, may reduce PTPN2 signaling via reduction of PTPN2 activity or PTPN2 expression in the cell. For example, the cell can be cultured in a suitable medium, to which a PTPN2 inhibitor is introduced for period of time sufficient to affect such reduction (or inhibition). Depending on the choice of the type of PTPN2 inhibitor, the contacting step may be affected by direct physical contact, pressure (e.g. by changing the shape of the cell via squeezing), chemical means (e.g., liposomes for delivery of nucleic acid based PTPN2 inhibitors), or viral means (e.g., when delivering shRNA, siRNA, or CRISPR-based PTPN2 inhibitors). The PTPN2 inhibitor may directly be introduced to a subject lymphoid cell ex vivo or in vitro. In some embodiments, the cell can be in a subject, and the PTPN2 inhibitor may be administered (e.g., systemically administered) to the subject to contact the cell in vivo. Upon such administration, at least a portion of the PTPN2 inhibitor may contact a cell (e.g., a lymphoid cell, a cancer, or tumor cell, etc.) of the subject in vivo. A composition (e.g., a therapeutic regimen) comprising the PTPN2 inhibitor may be administered to a target site comprising the cell (e.g., the cell may be part of the vascular or lymphatic system of the subject, or a localized tissue of interest or tumor). Alternatively or in addition to, the composition comprising the PTPN2 inhibitor may be administered to a different site than the target site. Upon such administration, the PTPN2 inhibitor may be directed to the target site or the cell via diffusion or via a medium such as a bodily fluid (e.g., blood).

When contacting a cell (e.g., a lymphoid cell) with the PTPN2 inhibitor ex vivo, the cell may be treated with a composition (e.g., a solution) comprising the PTPN2 inhibitor for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, or more, or any time in between. The cell may be treated with the composition comprising the PTPN2 inhibitor for at most 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or any time in between. During the contacting period, the cell may be subjected to additional PTPN2 inhibitor (e.g., to compensate for a limited half-life of the PTPN2 inhibitor in culture media). Alternatively, during the contacting period, the cell may not be subjected to any additional PTPN2 inhibitor. A process of contacting the cell with the PTPN2 inhibitor (e.g., treating the cell with a composition comprising the PTPN2 inhibitor) may be performed at least 1, 2, 3, 4, 5, or more times. In other embodiments, such process may be performed at most 5, 4, 3, 2, or 1 time.

In some embodiments, the cell as provided herein may retain expression or activity of PTPN2 prior to contacting (e.g., in vivo or ex vivo) the cell with the PTPN2 inhibitor. In some cases, any one of the methods disclosed herein may involve assessing the expression or activity of PTPN2 in the cell prior to contacting the cell with the PTPN2 inhibitor. In some examples, the cell may not exhibit any loss of the expression or activity of PTPN2, as compared to that present in a control sample, derived from e.g., another cell of the same origin of the cell or a progeny of the cell. In other examples, the cell may exhibit an expression or activity level of PTPN2 that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In yet some examples, the PTPN2 mRNA level, cDNA level, or PTPN2 polypeptide level expressed in the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, the cell may exhibit an activity level of PTPN2 (e.g., a degree of dephosphorylation of a target substrate) that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, an amount of PTPN2-associated cfDNA or cfRNA level within a source of the cell (e.g., from a plasma of a subject from whom/which the cell was obtained or derived from) may be indicative of an expression level of PTPN2 in the cell. As such, the amount of PTPN2-associated cfDNA or cfRNA level within a source of the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, e.g., another healthy subject who does not comprise or is not suspected of having a condition or disease of interest.

For any cell that is administered to a subject in need thereof, either with or without having been treated with a PTPN2 inhibitor as provided in the present disclosure, the cell may be autologous or allogenic to the subject. The cell may have been obtained from the subject and treated ex vivo (e.g., contacting with the PTPN2 inhibitor, engineered to express (i) the TFG and/or (ii) the CAR, etc.) prior to the administration. Alternatively, the cell may be a progeny of a cell obtained from the subject, and the progeny may have been treated ex vivo (e.g., contacting with the PTPN2 inhibitor, engineered to express (i) the TFG and/or (ii) the CAR, etc.) prior to the administration. In a different alternative, the cell may be a progeny of a cell obtained from the subject, and the progeny may be administered to the subject without any engineering or modification thereof. In other embodiments, the cell may be heterologous to the subject. In some examples, the cell may be an allogeneic cell, derived from, e.g., another human subject.

Any one of the subject methods disclosed herein may further comprise administering a PTPN2 inhibitor to the subject sequentially (e.g., prior to or subsequent to) or concurrent with administering a cell (e.g., a lymphoid cell) to the subject. In some embodiments, the cell may have been at least contacted previously with a PTPN2 inhibitor and, optionally, express the TFP and/or the CAR. In other embodiments, the cell may not have been contacted previously with a PTPN2 inhibitor and, optionally, express the TFP and/or the CAR. When introduced sequentially, the PTPN2 inhibitor and the cell may be administered by the same route (e.g. injections to the same location; tablets taken orally at the same time), or separately by a different route (e.g. a tablet taken orally while receiving an intravenous infusion). When introduced concurrently, the PTPN2 inhibitor and the cell may be, e.g., part of the same composition (e.g., the same condition media or a therapeutic regimen).

Any one of the subject methods disclosed herein may further comprise administering a lymphoid cell to the subject sequentially (e.g., prior to or subsequent to) or concurrent with administering a PTPN2 inhibitor to the subject. The lymphoid cell may optionally comprise (i) the chimeric T-cell receptor sequence and/or (ii) the CAR sequence. When introduced sequentially, the PTPN2 inhibitor and the lymphoid cell may be administered by the same route (e.g. injections to the same location; tablets taken orally at the same time), or by a different route (e.g. a tablet taken orally while receiving an intravenous infusion). When introduced concurrently, the PTPN2 inhibitor and the cell may be, e.g., part of the same composition (e.g., the same condition media or a therapeutic regimen). As described elsewhere in the present disclosure, the subject being administered a PTPN2 inhibitor can retain, prior to the administration of the PTPN2 inhibitor, expression or activity of PTPN2 in the subject's cells, such as lymphoid cells (e.g., T cells, NK cells, HKGY cells, and B cells), cancer cells, or tumor cells.

In practicing any one of the methods disclosed herein, selecting the subject may be based on one or more thresholds of an expression or activity level of PTPN2 in the subject's cells, such as lymphoid cells including, without limitation, effector cells such as T cells, NK cells, HKGY cells, and B cells, cancer cells, or tumor cells. For example, the subject's lymphoid cells, cancer cells, or tumor cells exhibit a PTPN2 expression or activity level in his or her lymphoid cells, cancer cells, or tumor cells that is at least 30%, 40%, 50%, 60%, 70%, 80%, 90%, 95%, or more of that present in a control sample. In some examples, the PTPN2 mRNA level or cDNA level expressed in the subject's lymphoid cells, cancer cells, or tumor cells is at least 30%, 40%, 50%, 60%, 70%, 80%, 90%, 95%, or more of that present in a control sample. In some examples, the PTPN2 or PTPN2-associated cfDNA or cfRNA level from the subject's lymphoid cells, cancer cells, or tumor cells is at least 30%, 40%, 50%, 60%, 70%, 80%, 90%, 95%, or more of that present in a control sample. In some examples, the subject's lymphoid cells, cancer cells, or tumor cells carry two copies or least one copy of PTPN2 genomic DNA. In some examples, the PTPN2 polypeptide level expressed in the subject's lymphoid cells is at least 30%, 40%, 50%, 60%, 70%, 80%, 90%, 95%, or more of that present in a control sample. In some examples, the subject's lymphoid cells, cancer cells, or tumor cells exhibit a normal level of expression or activity of PTPN2 as compared to that of a control sample. In some cases, selecting the subject that exhibits expression or activity of PTPN2 results in a negative selection against subject that does not express or possess functional PTPN2 as PTPN2-null phenotype, such that the step of downregulating (e.g., transiently downregulating or permanently downregulating) expression or activity of PTPN2 will not be performed.

The control sample utilized in assessing the PTPN2 expression level can be a biological sample from a subject that does not exhibit a tumor or cancer, or from a subject that has not been diagnosed with a tumor or cancer and that has not been treated with a PTPN2 inhibitor. Such control sample can comprise PTPN2 polynucleotides or PTPN2 polypeptides from any of such subject's tissues or cells, including but not limited to such subject's lymphoid cells.

In some embodiments, downregulating (e.g., transiently downregulating or permanently downregulating) expression or activity of PTPN2 in the cell of the subject may be performed in vivo. In some cases, as described elsewhere in the present disclosure, the cell of the subject may be contacted by a PTPN2 inhibitor in vivo by administering the PTPN2 inhibitor to the subject comprising the cell. Administering a PTPN2 inhibitor to a subject disclosed herein can stimulate or prolong anti-tumor or anti-cancer immunity. In other embodiments, downregulating expression or activity of PTPN2 in the cell of the subject may be performed in vivo. In some cases, as described elsewhere in the present disclosure, the cell of the subject may be isolated from the subject and may be contacted by a PTPN2 inhibitor ex vivo, e.g., treated with a composition comprising the PTPN2 inhibitor.

In practicing any one of the methods disclosed herein, administering a cell (e.g., an autologous or allogeneic lymphoid cell that optionally expresses a TFP and/or a CAR) to the subject may be performed sequentially (e.g., prior to or subsequent to) or concurrent with downregulating (e.g., transiently downregulating or permanently downregulating) expression or activity of PTPN2 in the cell. In some embodiments, the downregulating may comprise introducing a PTPN2 inhibitor to the cell, as provided in the present disclosure (e.g., contacting the cell with a PTPN2 inhibitor, or inducing the cell to express a PTPN2 inhibitor). When performed sequentially, a PTPN2 inhibitor and the cell may be introduced to the subject by the same route (e.g. injections to the same location; tablets taken orally at the same time), or by a different route (e.g. a tablet taken orally while receiving an intravenous infusion). When performed concurrently, a PTPN2 inhibitor and the cell may be, e.g., part of the same composition (e.g., the same condition media or a therapeutic regimen).

In some embodiments, a cell (e.g., a lymphoid cell, a cancer or tumor cell, etc.) of the subject may not exhibit a genetic alteration (e.g., mutation) of (i) a first gene encoding PTPN2 or (ii) a second gene operatively linked to PTPN2, wherein the genetic alteration reduces (or substantially inhibits) the expression and/or activity of PTPN2. In some examples, the second gene may be a promoter operatively linked to PTPN2 or an intron operatively linked to a gene product of PTPN2. Genetic alterations can include a mutation in a polynucleotide (e.g., DNA or RNA) encoding PTPN2 gene product. The mutation can affect any portion of the PTPN2 gene. The one or more PTPN2 mutations can include a mutation in the protein. The one or more PTPN2 mutations can be a point mutation, an insertion, a deletion, an amplification, a translocation, an inversion, or loss of heterozygosity. In some embodiments, the mutation is a loss of function. In some embodiments, the loss of function yields a dominant negative mutation. A mutation can be a frameshift mutation. A frameshift mutation can disrupt the reading frame, resulting in a completely different translated protein as compared to the original sequence. The mutation can be a nonsense mutation. The nonsense mutation can result in a premature stop codon, thus encoding a truncated, and possibly nonfunctional protein product. The PTPN2 mutation can be a nonsense mutation, wherein a single nucleotide alteration causes an amino acid substitution in the translated protein. The mutation can cause an alteration in one or more domain of the PTPN2 protein. The mutation can reduce binding efficacy of a PTPN2 protein with a PTPN2 substrate such as INSR, EGFR, CSF1R, PDGFR, JAK1, JAK2, JAK3, Src family kinases, STAT1, STAT3, STAT6, FYN, LCK, variations thereof, or combinations thereof. The mutation can reduce the ability of PTPN2 to dephosphorylate any one of the substrates disclosed herein, or reduce the ability of PTPN2 to interact with its upstream, or a downstream signaling molecules.

A method of potentiating immunity of a subject may comprise administering a lymphoid cell to the subject sequentially (e.g., prior to or subsequent to) and/or concurrent with the downregulation with the PTPN2 inhibitor. In some embodiments, contacting the lymphoid cell with a PTPN2 inhibitor may be performed in vivo, e.g., via administration of the PTPN2 inhibitor to the subject. In some cases, the subject may already comprise the lymphoid cell when the PTPN2 inhibitor is administered to the subject. The lymphoid cell may be an endogenous cell of the subject. Alternatively, the lymphoid cell may be a heterologous lymphoid cell (e.g., an allogeneic cell from a donor or a xenograft cell). In other cases, the subject may not comprise the lymphoid cell when the PTPN2 inhibitor is administered to the subject. Instead, the contact between the PTPN2 inhibitor and the lymphoid cell may occur upon administration of the lymphoid cell to the subject subsequent to the administration of the PTPN2 inhibitor to the subject. In some embodiments, contacting the lymphoid cell with a PTPN2 inhibitor may be performed ex vivo, e.g., in an in vitro culture composition. The lymphoid cell of the subject may be subjected to ex vivo expansion (or cell proliferation) prior to, during, or subsequent to being contacted by the PTPN2 inhibitor. When the resulting lymphoid cell and/or a progeny thereof is administered to the subject, the lymphoid cell and/or the progeny thereof may be washed to be substantially free of the PTPN2 inhibitor. Alternatively, the lymphoid cell and/or the progeny may not or need not be washed to rid of any excess, used, or expressed PTPN2 inhibitor prior to the administration to the subject.

In some embodiments, the method may further comprise introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the contacting of the lymphoid cell by the PTPN2 inhibitor may be performed sequentially (e.g., prior to or subsequent to) or concurrent with the introducing to the lymphoid cell the chimeric T-cell receptor sequence and/or the CAR sequence. In some examples, the lymphoid cell may be contacted with a PTPN2 inhibitor prior to being conditioned to express the TFP and/or the CAR. In other examples, the lymphoid cell may be contacted with a PTPN2 inhibitor while being conditioned to express the TFP and/or the CAR. In different examples, the lymphoid cell may be configured to express the TFP and/or the CAR prior to being contacted with a PTPN2 inhibitor.

In some embodiments, the downregulation of the expression or activity of PTPN2 in the lymphoid cell of the subject may be permanent. In other embodiments, as disclosed herein, the downregulation of the expression or activity of PTPN2 in a cell (e.g., the lymphoid cell of the subject) may comprise transiently downregulating the expression or activity of PTPN2. In some cases, downregulating the expression or activity of PTPN2 in the lymphoid cell performed sequentially (e.g., prior to or subsequent to) or concurrent with the introducing to the lymphoid cell the chimeric T-cell receptor sequence and/or the CAR sequence. In some examples, the expression or activity of PTPN2 in the lymphoid cell may be downregulated (e.g., with a PTPN2 inhibitor) prior to being conditioned to express the TFP and/or the CAR. In other examples, the expression or activity of PTPN2 in the lymphoid cell may be downregulated (e.g., with a PTPN2 inhibitor) while being conditioned to express the TFP and/or the CAR. In different examples, the lymphoid cell may be configured to express the TFP and/or the CAR prior to downregulating the expression or activity of PTPN2 in the lymphoid cell (e.g., with a PTPN2 inhibitor).

In some embodiments, a CAR of the present disclosure contains a minimally required intracellular signaling domain capable of activating a signaling cascade (e.g., an immunoreceptor signaling cascade) of the cell (e.g., in a lymphoid cell) in comparison to a control cell that is (i) without the CAR and/or (ii) in absence of any CAR activation (e.g., in absence of any antigen of the antigen-binding domain of the CAR). A minimally required intracellular signaling domain of the CAR typically consists of a primary signaling domain and lacks a co-stimulatory signaling domain sequence or a functional co-stimulatory signaling domain, and hence exhibiting less potency in activating an immune signaling cascade as compared to one with the co-stimulatory signaling domain. In some examples, the CAR with a minimally required intracellular signaling domain is a first-generation CAR. In some examples, the first-generation CAR contains only a primary signaling domain selected from the group consisting of CD3zeta, CD28, 4-1BB, OX40, DAP10, ICOS, and a variant thereof. In some examples, the CAR with a minimally required intracellular signaling domain is a second-generation CAR. In some examples, the second-generation CAR contains only a primary signaling domain selected from the group consisting of CD3zeta, CD28, 4-1BB, OX40, DAP10, ICOS, and a variant thereof, and a co-stimulatory signaling domain that is a different member from the primary signaling domain. In some examples, a cell comprising the CAR with the minimally required intracellular signaling domain may induce a target activity of the cell of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, or more than that of a control cell. In some examples, a cell comprising the CAR with the minimally required intracellular signaling domain may induce a target activity of the cell of at most about 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% or less than that of a control sample comprising a CAR with a more potent intracellular signaling domain. The more potent intracellular signaling domain may comprise a different polypeptide sequence (e.g., a polypeptide fragment derived from a different intracellular protein than the minimally required intracellular signaling domain) or an additional polypeptide sequence (e.g., the minimally required intracellular signaling domain plus one or more additional intracellular signaling domains). The additional polypeptide sequence may comprise at least 1, 2, 3, 4, 5, or more different intracellular signaling domains. Without wishing to be bound by theory, use of a CAR with the minimally required intracellular signaling domain may help to lower toxicity of a cell (e.g., a lymphocyte) expressing the CAR and/or increase persistence of the cell in the body of the subject in need of such cell therapy. In some cases, the use of PTPN2 inhibitor in conjunction with CAR-T therapy obviates the need to use other CAR-T cell proliferation inhibitors to control the toxicities inherent in CAR-T therapy. Non-limiting CAR-T cell proliferation inhibitors are specific protein kinase inhibitors such as INSR, EGFR, CSF1R, PDGFR, JAK1, JAK2, JAK3, Src family kinases, STAT1, STAT3, STAT6, FYN, LCK, variations thereof, or combinations thereof. In some embodiments, the methods disclosed herein obviate the need to utilize Nintedanib, Dasatinib, Saracatinib, Ponatinib, Nilotinib, Danusertib, AT9283, Degrasyn, Bafetinib, KW-2449, NVP-BHG712, DCC-2036, GZD824, GNF-2, PD173955, GNF-5, Bosutinib, Gefitinib, Erlotinib, and/or Sunitinib in conjunction of a CAR-T therapy. Another advantage of using PTPN2 inhibitor in conjunction with CAR-T therapy is that the amount of CAR-T cells required to yield a comparable level of in vivo efficacy is reduced. In some cases, a subtherapeutic amount of CAR-T cells is infused into a subject in need thereof. For example, one, two, or three orders of magnitude less of CAR-T cells are needed for treating a subject in need thereof. Where desired, less than 5×10⁶, 1×10⁶, 5×10⁵, 1×10⁵, 5×10⁴, 1×10⁴ CAR-T cells are needed to yield a comparable level of therapeutic effect as compared to a CAR-T therapy without the use of a PTPN2 inhibitor.

In practicing any one of the methods disclosed herein, examples of the target activity of the cell may include, but are not limited to, cytokine secretion, gene expression, cell proliferation, cytotoxicity against a target cell, cell death, chemotaxis, cellular metabolism, and/or cell exhaustion.

In practicing any one of the methods disclosed herein, a cell to be administered (e.g., systemically administered) may retain expression or activity of PTPN2 prior to administering a PTPN2 inhibitor to the subject. In some examples, a PTPN2 inhibitor may be administered to the subject prior to the administration of the cell, and the cell may be administered and contacted by the PTPN2 inhibitor in vivo to affect downregulation (e.g., transient downregulation) of expression or activity of PTPN2 in the cell in vivo. In other examples, a PTPN2 inhibitor and the cell may be administered at the same time, e.g., in a same composition or in different compositions, and the cell may be contacted by the PTPN2 inhibitor ex vivo and/or in vivo to affect downregulation of expression or activity of PTPN2 in the cell. In different examples, a PTPN2 inhibitor may be administered to the subject subsequent to the administration of the cell to the subject, and the cell may be contacted by the PTPN2 inhibitor in vivo to affect downregulation of expression or activity of PTPN2 in the cell in vivo.

In practicing any of the methods disclosed herein, the PTPN2 inhibitor may be a compound disclosed herein, such as a compound of Formula (I), (Ia), (I-1), (I-1a), (I-2), (I-2a), (I-A), (I-Aa), (I-A1), (I-A1a), (I-A2), (I-A2a), (I-B), (I-Ba), (I-B1), (I-B1a), (I-B2), (I-B2a), (I-C), (I-Ca), (I-C1), (I-C1a), (I-C2), or (I-C2a), or a pharmaceutically acceptable salt or solvate thereof.

In practicing any one of the methods disclosed herein, a therapeutic amount or an effective amount may be an amount of a composition or a pharmaceutical formulation (e.g., a cell, a PTPN2 inhibitor, etc.) that is sufficient to elicit a desired response in the subject upon a treatment or method of the present disclosure. In some embodiments, a sub-therapeutic amount of a composition or a pharmaceutical formulation may be an amount of the composition or pharmaceutical formulation that is a fragment of the therapeutic amount. In some examples, a sub-therapeutic amount of a cell (e.g., a cell expression the CAR) may comprise a cell number that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a cell number of a therapeutic amount. For example, one, two, or three orders of magnitude less of CAR-T cells that are normally required absent of the use of PTPN2 inhibitor are contemplated for administering into a subject in need thereof. Where desired, a sub-therapeutic amount of cells such as 5×10^{6, 1}×10⁶, 5×10⁵, 1×10⁵, 5×10⁴, or 1×10⁴ CAR-T cells are needed to yield a comparable level of therapeutic effect as compared to a CAR-T therapy without the use of a PTPN2 inhibitor.

In some examples, a sub-therapeutic amount of a drug (e.g., a PTPN2 inhibitor) may comprise a dose of the drug that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a dose of the drug of a therapeutic amount. Without wishing to be bound by theory, use of a sub-therapeutic amount (or dose) of a cell expressing the CAR may help to lower toxicity of such cell therapy and/or increase persistence of the cell in the body of the subject in need of such cell therapy.

In practicing any one of the methods disclosed herein, the immunity of a cell or a subject may be anti-tumor, anti-cancer activity, anti-viral infection activity, and/or anti-bacterial infection activity. In some embodiments, examples of a viral infection and bacterial infection may comprise human bacterial, human parasitic protozoan or human viral infections caused by microbial species including Plasmodium, Pneumocystis, herpes viruses (CMV, HSV 1, HSV 2, VZV, and the like), retroviruses, adenoviruses, and the like. In some examples, any one of the subject methods of the present disclosure may be used to treat or regulate HIV infections and related conditions such as tuberculosis, malaria, Pneumocystis pneumonia, CMV retinitis, AIDS, AIDS-related complex (ARC) and progressive generalized lymphadenopathy (PGL), and AIDS-related neurological conditions such as multiple sclerosis, and tropical spastic paraparesis. Other human retroviral infections that may be treated or regulated by any one of the subject methods of the present disclosure include Human T-cell Lymphotropic virus and HIV-2 infections.

In embodiments, when practicing any one of the methods disclosed herein, the PTPN2 inhibitor does not regulate site-specific recombination of a gene encoding PTPN2. In some examples, the gene encoding PTPN2 or a gene operatively linked to the gene encoding PTPN2 (e.g., a transcription factor, an intron sequence, etc.) may not be flanked by a recombinase site (e.g., Cre recombinase or Flp recombinase substrates). In some examples, the PTPN2 inhibitor may not be an activator of recombination of a recombinase site. In an example, the PTPN2 inhibitor may not be an estrogen antagonist.

In practicing any one of the methods disclosed herein, the PTPN2 expression or activity level can be determined by detecting the PTPN2 polynucleotides or PTPN2 polypeptides present in a cell or tissue. A wide variety of nucleic acid assays are available for detecting and/or quantifying PTPN2 polynucleotides, including PTPN2 DNAs and PTPN2 RNAs. Exemplary nucleic acid assays include but are not limited to genotyping assays and sequencing methods. Sequencing methods can include next-generation sequencing, targeted sequencing, exome sequencing, whole genome sequencing, massively parallel sequencing, and the like.

Additional methods for assessing levels and/or concentration of PTPN2 polynucleotides in a tissue or a cell may include, but are not limited to, microarray hybridization assay, nucleic acid amplification assays including without limitation polymerase chain reaction (PCR), quantitative PCR (qPCR), real-time PCR (RT-PCR), digital PCR, and in situ sequencing (US20190024144, US20140349294, incorporated hereby by reference). Nucleic acid amplification can be linear or non-linear (e.g., exponential). Amplification may comprise directed changes in temperature or may be isothermal. Conditions favorable to the amplification of target sequences by nucleic acid amplification assays are known in the art, can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, some or all of which can be altered. In situ hybridization (ISH), RNase protection assay, and the like assays can also be employed for detecting PTPN2 polynucleotides and the expression level.

In some embodiments, the copy number PTPN2 gene is assessed by a method selected from the group consisting of in situ hybridization (ISH), Southern blot, immunohistochemistry (IHC), polymerase chain reaction (PCR), quantitative PCR (qPCR), quantitative real-time PCR (qRT-PCR), comparative genomic hybridization (CGH), microarray-based comparative genomic hybridization, and ligase chain reaction (LCR). In some embodiments, the in situ hybridization is selected from fluorescence in situ hybridization (FISH), chromogenic in situ hybridization (CISH) and silver in situ hybridization (SISH). In some embodiments, the copy number is assessed using a nucleic acid sample from the subject, such as genomic DNA, cDNA, ctDNA, cell-free DNA, RNA or mRNA.

PTPN2 expression and/or activity level can also be assessed by detecting and/or quantifying PTPN2 polypeptide level in a subject's tissue or cell. A variety of techniques are available in the art for protein analysis. They include but are not limited to immunohistochemistry (IHC), radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, confocal microscopy, enzymatic assays, surface plasmon resonance and PAGE-SDS. One or more of these protein assays utilizes antibodies or fragments thereof that exhibits specific binding to PTPN2 polypeptides. A large number of anti-PTPN2 antibodies are available, including those provided by Invitrogen, Santa Cruz Biotechnology, OriGene Technologies, MilliporeSigma, Bio-Rad, Abcam, and Cell Signaling Technology.

In practicing any one of the subject methods as provided herein, the PTPN2 expression or activity, e.g., in a tumor tissue, a cancer cell, or a lymphoid cell, can be determined using any biological sample comprising the target cells (e.g., plasma cells or cells from a tumor site under investigation) or constituents thereof (e.g., constituents such as cfDNA from the plasma or the tumor site). The biological sample may be a solid or liquid biological sample from the subject under investigation or treatment. The biological sample may be a biopsy sample that is fixed, paraffin-embedded, fresh, or frozen. The biological sample may be obtained by any suitable means, including but not limited to needle aspiration, fine needle aspiration, core needle biopsy, vacuum assisted biopsy, large core biopsy, incisional biopsy, excisional biopsy, punch biopsy, shave biopsy, skin biopsy, and venipuncture.

The biological sample can be obtained from, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues of the subject. In some embodiments, a selection of the biological sample may depend on the condition of the subject to be treated.

In some embodiments, a biological sample comprises cell-free DNA (cfDNA) derived from a whole blood or plasma of the subject. A sample may be analyzed directly for its contents or may be processed to purify one or more of its contents for analysis. Methods of direct analysis of samples are known in the art and include, without limitation, mass spectrometry and histological staining procedures. In some embodiments, one or more components are purified from the sample for the detection of PTPN2 expression level or activity level. In some embodiments, the purified component of the biological sample is protein (e.g. total protein, cytoplasmic protein, or membrane protein). In some embodiments, the purified component of the sample is a nucleic acid, such as DNA (e.g. genomic DNA, cDNA, ctDNA, or cfDNA) or RNA (e.g. total RNA or mRNA).

In some embodiments, as abovementioned, the cell may be contacted by a PTPN2 inhibitor in vivo by administering the PTPN2 inhibitor to the subject comprising the cell. Administering a PTPN2 inhibitor to a subject disclosed herein can stimulate or prolong anti-tumor or anti-cancer immunity. Not wishing to be bound by any particular theory, a PTPN2 inhibitor reduces PTPN2 activity in a cell, leading to an augmented immunoreceptor signaling pathways, which in turn results in the activation of adaptive immunity against tumor or cancer cells.

Stimulation of anti-tumor or anti-cancer immunity can be established by any readout known in the art including without limitation: lymphoid cell proliferation (including proliferation of T cells such as CD4+ and/or CD8+ T cells, and clonal expansion other lymphoid cells), cytokine secretion, activation of effector function of lymphoid cells, reduction in T cell exhaustion, destabilization of regulatory T cells (Tregs) and/or their function, movement and/or trafficking of lymphoid cells, release of other intracellular signaling molecules, and phosphorylation of intracellular signaling molecules.

In some embodiments, anti-tumor immunity encompasses proliferation of the lymphoid cells including clonal expansion of the lymphoid cells that are capable of directly or indirectly mediating anti-tumor activity. Non-limiting examples of anti-tumor lymphoid cells are CD4+ and/or CD8+ T cells, NK cells, tumor infiltrating lymphocytes (TIL), especially those T cells capable of specific binding to one or more tumor antigens. Proliferation of the lymphoid cell can lead to a phenotypic change of the lymphoid cell. Treatment of a PTPN2 inhibitor can stimulate or prolong lymphoid cell proliferation by about 1 fold, about 2 to about 5 fold, about 5 to about 10 fold, about 10 fold to about 50 fold, about 50 fold to about 100 fold or higher. Assessing lymphoid cell proliferation can be performed by a wide variety of assays known in the art, including without limitation, the use of cell staining, microscopy, flow cytometry, cell sorting, and combinations of these. A number of commercial kits for assessing various types of T cell or B cell proliferations are also suitable to assess the effect of PTPN2 inhibitor on T cell or B cell proliferation (e.g., IncuCyte, CellTRrace Cell Proliferation Kits marketed by ThermoFisher). Proliferation can also be determined by phenotypic analysis of the lymphoid cells. For example, clumping of lymphoid cells in culture can signify proliferation of lymphoid cells as compared to comparable lymphoid cells without the treatment with a PTPN2 inhibitor.

In some embodiments, anti-tumor immunity stimulated or prolonged in response to a PTPN2 inhibitor is evidenced by cytokine release from the lymphoid cells. Cytokine release by the lymphoid cell can comprise the release of IFNγ, TNFα, CSF, TGFβ, IL-1, IL-2, IL-4, IL-5, IL-6, IL-13, IL-17, IL-21, IL-22, granzyme, and the like. Lymphoid cells can generate about 1 fold, 2 fold, 3 fold, 4 fold, 5 fold, 6 fold, 7 fold, 8 fold, 9 fold, 10 fold, 50 fold, 100 fold or greater cytokine release in response to a PTPN2 inhibitor treatment as compared to comparable lymphoid cells that are not being exposed to the PTPN2 inhibitor. Cytokine release may be determined and quantified using any immunoassays such as western blot, ELISA, flow cytometry, and the like.

In some embodiments, stimulated or prolonged anti-tumor immunity is evidenced by T cell activation. T cell activation can involve differential expression of antigen specific TCRs, certain cell surface markers and induction of cell proliferation signals. T cell activation may also involve stimulating its effector function including cytolytic activity against tumor or cancer cells, or helper activity including releasing cytokines. In some examples, T cells can be used to kill a tumor or cancer cell in vivo or in vitro in the presence of a PTPN2 inhibitor. Cell killing can be mediated by the release of one or more cytotoxic cytokines, for example IFNγ or granzyme, by the T cells. In some cases, a subject method can stimulate or prolong the (i) release of cytotoxins such as perforin, granzymes, and granulysin and/or (ii) induction of apoptosis via e.g., Fas-Fas ligand interaction between the T cells and a tumor or cancer cell, thereby triggering the destruction of the target cell. Cytotoxicity can be detected by staining, microscopy, flow cytometry, cell sorting, ELISPOT, chromium release cytotoxicity assay, and other cell death assays described in WO2011131472A1, which is incorporated herein by reference.

Cytotoxicity of a lymphoid cell can be greater in response to treating with a PTPN2 inhibitor as compared to a comparable lymphoid cell lacking such treatment. A lymphoid cell treated with a PTPN2 inhibitor can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to a comparable lymphoid cell lacking the treatment. In some embodiments, a change in cytotoxicity can comprise comparing such activity before and after treating the lymphoid cell with a PTPN2 inhibitor.

In some examples, a reduction in expression or activity of such markers including PD1, Foxp3, or FoxO3a is indicative of Treg destabilization, and hence an enhanced anti-tumor immunity. In addition, Treg destabilization, as reflected by a decreased T cell exhaustion, can be demonstrated by an enhanced cytokine release, e.g., release of IL-2, IFNγ, TNF and other chemokines.

Anti-tumor immunity can also be evidenced by movement and/or trafficking of the lymphoid cells in response to a treatment with a PTPN2 inhibitor. In some embodiments, movement can be determined by quantifying localization of the lymphoid cell to a target site such as a tumor tissue. For example, lymphoid cells can be quantified at the target before or after administration of a PTPN2 inhibitor. Quantification can be performed by isolating a lesion and quantifying a number of lymphoid cells, for example tumor infiltrating lymphocytes. Movement and/or trafficking of lymphoid cells in a tumor tissue after administering a PTPN2 inhibitor can be greater than that of a control lacking the administration of a PTPN2 inhibitor. In some embodiments, the number of lymphoid cells accumulated at the tumor tissue of interest can be about 1 fold, 5 fold, 10 fold, 15 fold, 50 fold, 100 fold or greater than that of a control not being treated with a PTPN2 inhibitor. Trafficking can also be determined in vitro utilizing a transwell migration assay. In some embodiments, the number of lymphoid cells administered with a PTPN2 inhibitor exhibits about 1 fold, 5 fold, 10 fold, 15 fold, 50 fold, 100 fold or greater as compared to that of control lymphoid cells not being administered with a PTPN2 inhibitor.

Stimulating and/or prolonging anti-tumor immunity in a subject can also be assessed by one or more (in any combination) of the foregoing results, although alternative or additional results of the referenced tests and/or other tests can evidence such desired outcome. In some embodiments, anti-tumor immunity is considered stimulated if there exists at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000%, 10000% or more improvement, using an appropriate measure (e.g. tumor size reduction, duration of tumor size stability, duration of time free from metastatic events, duration of disease-free survival). Improved immunity may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or more, using an appropriate measure (e.g. tumor size reduction, duration of tumor size stability, duration of time free from metastatic events, duration of disease-free survival).

A number of secondary parameters can be employed to determine stimulated and/or prolonged anti-tumor immunity.

Examples of secondary parameters include, but are not limited to, the lack of new tumors, a reduction of circulating tumor antigens or markers (e.g., CEA, PSA, CA-125, or cfDNA, ctDNA), the lack of detectable cancer cell or tumor marker by way of biopsy, surgical downstaging (i.e., conversion of the surgical stage of a tumor from unresectable to resectable), MRI, ultrasound, PET scans and any other detection means, all of which can point to the overall immunity to tumor or cancer in a subject. Examples of tumor markers and tumor-associated antigens that can be evaluated as indicators of improved immunity include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, gp100, Ki-67, STK15, Survivin, Cyclin B1, Stromelysin, Cathepsin L^{2, 3}MYBL2, and any ctDNA known in the art. BMC Med. 16:166, 2018.

In some embodiments, prolonged immunity is evidenced by tumor being stabilized (e.g., one or more tumors do not increase more than 1%, 5%, 10%, 15%, or 20% in size, and/or do not metastasize) as a result of treatment with a PTPN2 inhibitor. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95%, or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

The methods disclosed herein can be applied to treat, stimulate and/or or prolong immunity against a wide variety of cancers, including both solid tumor hematological cancers. For example, the subject methods can be applied to: Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Basal Cell Carcinoma of the Skin, Bile Duct Cancer, B1adder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma-see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sézary Syndrome), Ductal Carcinoma In Situ (DCIS), Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma (Soft Tissue Sarcoma), Kidney (Renal Cell) Cancer, Laryngeal Cancer (Head and Neck Cancer), Leukemia, Lip and Oral Cavity Cancer (Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma (Skin Cancer), Mesothelioma, Malignant, Metastatic Cancer, Metastatic Squamous Neck Cancer with Occult Primary (Head and Neck Cancer), Midline Tract Carcinoma, Mouth Cancer (Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary B1astoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and any of the aforementioned cancers exhibiting expression and/or activity of PTPN2 in the cancer cells.

Certain embodiments contemplate a human subject that has been diagnosed with a cancer, such as one in which PTPN2 expression or activity is detectable (e.g., aberrantly low, normal, or high) in the cancer cells or tumor tissue. Certain other embodiments contemplate a non-human subject, for example a non-human primate such as a macaque, chimpanzee, gorilla, vervet, orangutan, baboon or other non-human primate, including such non-human subjects that can be known to the art as preclinical models, the tumor tissue or cancer cells of which exhibit expression and/or activity of PTPN2. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present disclosure, a transgenic animal is utilized. A transgenic animal is a non-human animal in which one or more of the cells of the animal include a nucleic acid that is non-endogenous (i.e., heterologous) and is present as an extrachromosomal element in a portion of its cell or stably integrated into its germ line DNA (i.e., in the genomic sequence of most or all of its cells).

Where desired, the subject can be screened for the presence of expression or activity of PTPN2 in the subject's tumor or cancer cells. The subject can also be screened for the retention of PTPN2 expression and/or activity in one or more types of subject's lymphoid cells. Screening for the presence or the absence of expression or activity of PTPN2 can be carried out by analyzing the PTPN2 polynucleotide or PTPN2 polypeptide with any of the nucleic acid or protein assays disclosed herein. One or more of the screening steps can be performed concurrent with, subsequent to, or more likely, prior to administering a PTPN2 inhibitor to the subject.

The present disclosure also provides a cell (including a population of cells, such as a population of lymphoid cells) modified to express an exogenous sequence, and wherein expression and/or activity of PTPN2 in said cell has been inhibited (including reduction and elimination). In one aspect, provided in the disclosure is a lymphoid cell in which the expression and/or function of PTPN2 in said cell is inhibited. Such inhibition can be transient or permanent, occurring in vitro, ex vivo, or in vitro. In some cases, as used herein, inhibiting expression and/or function of a target molecule may be referred to downregulation of expression and/or function of the target molecule. A modified lymphoid cell of the present disclosure can be further characterized in that it comprises: (a) a chimeric T-cell receptor sequence encoding a T-cell receptor fusion protein (TFP), and/or (b) a chimeric antigen receptor (CAR) sequence encoding a CAR, wherein each of TFP and CAR exhibits specific binding to an antigen, including but not limited to a tumor or tumor-associated antigen.

Not wishing to be bound by any particular theory, inhibiting PTPN2 expression and/or activity of such lymphoid cell can lead to an augmented immunoreceptor signaling, which in turn results in the activation of an adaptive immunity against tumor or cancer cells. When its PTPN2 expression or activity is inhibited, the modified lymphoid cells can exhibit enhanced cell proliferation (including proliferation of T cells such as CD4+ and/or CD8+ T cells, and clonal expansion other lymphoid cells), enhanced cell activity (including e.g., cytokine secretion, activation of effector function, trafficking to tumor site or cancer cell), or enhanced disability (e.g., reduction in T cell exhaustion, destabilization of regulatory T cells (Tregs) in terms of cell number and cellular function).

In practicing any one of the methods disclosed herein, a subject cell (e.g., a modified cell such as a modified lymphoid cell) may comprise an enhancer moiety capable of enhancing one or more activities of the cell. In some embodiments, an enhancer moiety suitable for incorporating into a subject cell (e.g., a modified lymphoid cell) can be cytokines and growth factors capable of stimulating the growth, clonal expansion, and/or enhancing persistence of the immune cell in vivo. An enhancer may be intracellular, membrane-bound (e.g., a receptor or an adaptor protein of a receptor) or secreted by the cell. Encompassed are enhancer moieties selected from the group consisting of IL-2, IL-3, IL-4, IL-6, IL-7, IL-10, IL-11, IL-12, IL-15, IL-17, IL-18, IL-21, IL-23, PD-1, PD-L¹, CD122, CSF1R, CTAL-4, TIM-3, TGFR beta, receptors for the same, functional fragments thereof, functional variants thereof, and combinations thereof. An enhancer moiety may be expressed from an endogenous gene of the cell. Alternatively, or in addition to, an enhancer moiety may be expressed from a heterologous gene introduced to the cell. Such heterologous gene may be chromosomal (e.g., in the nuclear chromosome or mitochondrial chromosome) or epichromosomal. In some examples, a cell (e.g., a modified immune cell configured to express a TFP and/or a CAR) may be engineered such that one or more enhancer moieties are constitutively expressed and/or activated. In other examples, the one or more enhancer moieties may be transiently expressed for a limited time. In different examples, the one or more enhancer moieties may be conditionally expressed under, e.g., activation of a cellular signaling.

In practicing any one of the methods disclosed herein, a subject cell (e.g., a modified cell such as a modified lymphoid cell) may comprise an inducible cell death moiety, which inducible cell death moiety effects cell death (e.g., suicide) of the cell upon contact with a cell death activator. Where desired, an inducible cell death moiety is selected from the group consisting of: caspase-1 ICE, caspase-3 YAMA, inducible Caspase 9 (iCasp9), AP1903, HSV-TK, CD19, RQR8, tBID, CD20, truncated EGFR, Fas, FKBP12, CID-binding domain (CBD), and any combination thereof. Examples of further suicide systems include those described by Jones et al. (Jones BS, Lamb LS, Goldman F and Di Stasi A (2014) Improving the safety of cell therapy products by suicide gene transfer. Front. Pharmacol. 5:254. doi: 10.3389/fphar.2014.00254), which is incorporated herein by reference in its entirety. Where desired, a suitable inducible cell death moiety can be HSV-TK, and the cell death activator is GCV. Where further desired, a suitable inducible cell death moiety can be iCasp9, and the cell death activator is AP1903.

A TFP comprised in the subject lymphoid cell typically comprises a TCR subunit comprising (1) a TCR extracellular domain capable of specific binding to an antigen domain, and (2) an intracellular signaling domain. Upon expression of the TFP, it forms a T cell receptor (TCR) complex. In some embodiments, the TCR extracellular domain comprises (1) an antigen binding domain capable of specific binding to the antigen, and (2) an extracellular domain or portion thereof of a protein including, e.g., the alpha, beta or zeta chain of the T-cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154. In general, the antigen binding domain and the extracellular domain are operatively linked together, e.g., in the same reading frame.

In some embodiments, a subject CAR comprises an antigen-binding domain and an intracellular signaling domain. In some exmaples, the antigen-binding domain and the intracellular signaling domain of the CAR are linked via a transmembrane domain.

### Antigen Binding Domain of TFP or CAR

The antigen binding domain of a TFP or CAR disclosed herein typically comprises an antigen-specific binding element, the choice of which depends upon the type and number of antigens of interest. For example, the antigen binding domain may be chosen to recognize a cell surface marker on a target cell associated with a particular disease state. Non-limiting examples of cell surface markers include those associated with a tumor or cancer, with viral, bacterial and parasitic infections, autoimmune disease, inflammation diseases and metabolic disease. Cell surface markers can include, without limitation, carbohydrates, glycolipids, glycoproteins; CD (cluster of differentiation) antigens present on cells of a hematopoietic lineage (e.g., CD2, CD4, CD8, CD21, etc.), y-glutamyltranspeptidase, an adhesion protein (e.g., ICAM-1, ICAM-2, ELAM-1, VCAM-1), hormone, growth factor, cytokine, and other ligand receptors, ion channels, and the membrane-bound form of an immunoglobulin u chain.

Of particular interest are biological markers associated with a tumor or cancer or a stage or state of a cancer. A vast variety of disease-related biological markers have been identified, and the corresponding targeting moieties have been generated, including but not limited to cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis (a) antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdr1 gene product), multidrug-resistance-related antigen, p170, multidrug-resistance-related antigen, prostate specific antigen (PSA), CD56, and NCAM.

In some examples, the antigen binding domain of the subject TCR specifically binds to CD19. A large number of exemplary anti-CD19 antigen binding domains and constructs thereof are described in U.S. Pat. Nos. 8,399,645; 7,446,190; WO2012/079000; WO2014/031687; U.S. Pat. No. 7,446,190; each of which is herein incorporated by reference in its entirety. In some other examples, the antigen binding domain of the subject TCR specifically binds to BCMA. Exemplary anti-BCMA antigen binding domains and constructs thereof are described in e.g., WO2012163805, WO200112812, and WO2003062401, WO2016/014565, WO2014/122144, WO2016/014789, WO2014/089335, WO2014/140248, each of which is hereby incorporated by reference in its entirety. In some other examples, the antigen binding domain of the subject TCR specifically binds to CD123. Exemplary anti-CD123 antigen binding domains and constructs thereof are described in e.g., WO2014/130635, WO2016/028896, WO2008/127735, WO2014/138805, WO2014/138819, WO2013/173820, WO2014/144622, WO2001/66139, WO2010/126066, WO2014/144622, and US2009/0252742, each of which is incorporated herein by reference in its entirety. In yet some other examples, the antigen binding domain of the subject TCR specifically binds to CD38. Exemplary anti-CD38 antigen binding domains are embodied in daratumumab (described in e.g., Groen et al., Blood 116 (21): 1261-1262 (2010); MOR202 (see, e.g., U.S. Pat. No. 8,263,746); or antibodies described in U.S. Pat. No. 8,362,211.

In some other examples, the antigen binding domain of the subject TCR specifically binds to Tn antigen. Exemplary anti-Tn antigen binding domains and constructs thereof are described in e.g., US 2014/0178365, U.S. Pat. No. 8,440,798, Brooks et al., PNAS 107 (22): 10056-10061 (2010), and Stone et al., Oncolmmunology 1 (6): 863-873 (2012). In yet some other examples, the antigen binding domain of the subject TCR specifically binds to CS-1. Exemplary anti-CS-1 antigen binding domains and constructs thereof are described in Elotuzumab (BMS), see e.g., Tai et al., 2008, Blood 112 (4): 1329-37; Tai et al., 2007, Blood. 110 (5): 1656-63. In yet some other examples, the antigen binding domain of the subject TCR specifically binds to mesothelin. Exemplary anti-mesothelin antigen binding domain are described in, e.g., WO2015/090230, WO1997/025068, WO1999/028471, WO2005/014652, WO2006/099141, WO2009/045957, WO2009/068204, WO2013/142034, WO2013/040557, WO2013/063419, each of which is incorporated by reference in its entirety. In yet some other examples, the antigen binding domain of the subject TCR specifically binds to CD22. Exemplary anti-CD22 antigen binding domains are described in Haso et al., Blood, 121 (7): 1165-1174 (2013); Wayne et al., Clin Cancer Res 16 (6): 1894-1903 (2010), each of which is incorporated herein by reference. In yet some other examples, the antigen binding domain of the subject TCR specifically binds to CLL-1. Exemplary anti-CLL-1 antigen binding domains are described in WO2016/014535, incorporated herein by reference.

In yet some other examples, the antigen binding domain of the subject TCR specifically binds to CD33. Exemplary anti-CD33 antigen binding domains are described in WO2016/014576 and WO2016/014576, each of which is incorporated by reference in its entirety. In yet some other examples, the antigen binding domain of the subject TCR specifically binds to GD2. Exemplary anti-GD2 antigen binding domains are described in WO2012033885, WO2013040371, WO2013192294, WO2013061273, WO2013123061, WO2013074916, WO201385552, WO 2011160119, and US20100150910, each of which is incorporated by reference in its entirety. In yet some other examples, the antigen binding domain of the subject TCR specifically binds to PSMA. Exemplary anti-PSMA antigen binding domains are described in US20110268656 (J591 ScFv); WO 2006125481 (mAbs 3/A12, 3/E7 and 3/F11) and single chain antibody fragments (scFv A5 and D7), each of which is incorporated by reference in its entirety. In yet some other examples, the antigen binding domain of the subject TCR specifically binds to FLT3. Exemplary anti-FLT3 antigen binding domains are described in e.g., WO2011076922, U.S. Pat. No. 5,777,084, EP0754230, US20090297529, and several commercial catalog antibodies (R&D, ebiosciences, Abcam), each of which is incorporated by reference in its entirety. In yet some other examples, the antigen binding domain of the subject TCR specifically binds to ROR1. Exemplary anti-ROR1 antigen binding domains are described in WO 2011159847, US20130101607, each of which is incorporated by reference in its entirety. In yet some other examples, the antigen binding domain of the subject TCR specifically binds to TAG72. Exemplary anti-TAG72 antigen binding domains are described in Hombach et al., Gastroenterology 113 (4): 1163-1170 (1997); and Abcam ab691.

In yet some other examples, the antigen binding domain of the subject TCR specifically binds to FAP. Exemplary anti-FAP antigen binding domains are described in US 2009/0304718, incorporated herein by reference. In yet some other examples, the antigen binding domain of the subject TCR specifically binds to CD44v6. Exemplary anti-CD44v6 antigen binding domains are described in Casucci et al., Blood 122 (20): 3461-3472 (2013). In yet some other examples, an antigen binding domain against CEA is an antigen binding portion, e.g., CDRs, of an antibody described in, e.g., Chmielewski et al., Gastoenterology 143 (4): 1095-1107 (2012). In yet some other examples, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W⁹⁴; ING-1; and adecatumumab (MT201). In yet some other examples, an antigen binding domain against PRSS21 is an antigen binding portion, e.g., CDRs, of an antibody described in U.S. Pat. No. 8,080,650. In yet some other examples, an antigen binding domain against IL-13Ra2 is an antigen binding portion, e.g., CDRs, of an antibody described in, e.g., WO2008/146911, WO2004087758, several commercial catalog antibodies, and WO2004087758. In yet some other examples, an antigen binding domain against B7H3 is an antigen binding portion, e.g., CDRs, of an antibody MGA271 (Macrogenics). In yet some other examples, an antigen binding domain against KIT is an antigen binding portion, e.g., CDRs, of an antibody described in, e.g., U.S. Pat. No. 7,915,391, US20120288506, and several commercial catalog antibodies. In yet some other examples, an antigen binding domain against CD30 is an antigen binding portion, e.g., CDRs, of an antibody described in, e.g., U.S. Pat. No. 7,090,843 B1, and EP0805871. In yet some other examples, an antigen binding domain against GD3 is an antigen binding portion, e.g., CDRs, of an antibody described in, e.g., U.S. Pat. Nos. 7,253,263; 8,207,308; US20120276046; EP1013761; WO2005035577; and U.S. Pat. No. 6,437,098. In yet some other examples, an antigen binding domain against CD171 is an antigen binding portion, e.g., CDRs, of an antibody described in, e.g., Hong et al., J Immunother 37 (2): 93-104 (2014). In yet some other examples, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72 (1): 271-281 (2012). In yet some other examples, an antigen binding domain against PSCA is an antigen binding portion, e.g., CDRs, of an antibody described in, e.g., Morgenroth et al., Prostate 67 (10): 1121-1131 (2007)(scFv 7F5); Nejatollahi et al., J of Oncology 2013 (2013), article ID 839831 (scFv C₅-II); and US Pat Publication No. 20090311181. In yet some other examples, an antigen binding domain against VEGFR2 is an antigen binding portion, e.g., CDRs, of an antibody described in, e.g., Chinnasamy et al., J Clin Invest 120 (11): 3953-3968 (2010). In yet some other examples, an antigen binding domain against LewisY is an antigen binding portion, e.g., CDRs, of an antibody described in, e.g., Kelly et al., Cancer Biother Radiopharm 23 (4): 411-423 (2008)(hu3S193 Ab (scFvs)); Dolezal et al., Protein Engineering 16 (1): 47-56 (2003)(NC10 scFv). In yet some other examples, an antigen binding domain against CD24 is an antigen binding portion, e.g., CDRs, of an antibody described in, e.g., Maliar et al., Gastroenterology 143 (5): 1375-1384 (2012). In yet some other examples, an antigen binding domain against CD20 is an antigen binding portion, e.g., CDRs, of the antibody Rituximab, Ofatumumab, Ocrelizumab, Veltuzumab, or GA101. In yet some other examples, an antigen binding domain against PDGFR-beta is an antigen binding portion, e.g., CDRs, of an antibody Abcam ab32570. In yet some other examples, an antigen binding domain against SSEA-4 is an antigen binding portion, e.g., CDRs, of antibody MC813 (Cell Signaling), or other commercially available antibodies. In yet some other examples, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484. In yet some other examples, an antigen binding domain against ERBB2 (Her2/neu) is an antigen binding portion, e.g., CDRs, of the antibody trastuzumab, or pertuzumab. In yet some other examples, an antigen binding domain against MUC1 is an antigen binding portion, e.g., CDRs, of the antibody SAR566658. In yet some other examples, the antigen binding domain against EGFR is antigen binding portion, e.g., CDRs, of the antibody cetuximab, panitumumab, zalutumumab, nimotuzumab, or matuzumab. In one embodiment, the antigen binding domain against EGFRvIII is or may be derived from an antigen binding domain, e.g., CDRs, scFv, or VH and VL, of an antibody, antigen-binding fragment or CAR described in, e.g., PCT publication WO2014/130657 (In one embodiment the CAR is a CAR described in WO2014/130657, the contents of which are incorporated herein in their entirety). In yet some other examples, an antigen binding domain against NCAM is an antigen binding portion, e.g., CDRs, of the antibody clone 2-2B: MAB5324 (EMD Millipore). In yet some other examples, an antigen binding domain against Ephrin B2 is an antigen binding portion, e.g., CDRs, of an antibody described in, e.g., Abengozar et al., Blood 119 (19): 4565-4576 (2012). In yet some other examples, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995. In yet some other examples, an antigen binding domain against CAIX is an antigen binding portion, e.g., CDRs, of the antibody clone 303123 (R&D Systems). In yet some other examples, an antigen binding domain against LMP2 is an antigen binding portion, e.g., CDRs, of an antibody described in, e.g., U.S. Pat. No. 7,410,640, or US20050129701. In yet some other examples, an antigen binding domain against gp100 is an antigen binding portion, e.g., CDRs, of the antibody HMB45, NKIbetaB, or an antibody described in WO2013165940, or US20130295007. In yet some other examples, an antigen binding domain against tyrosinase is an antigen binding portion, e.g., CDRs, of an antibody described in, e.g., U.S. Pat. No. 5,843,674; or U.S. Ser. No. 08/504,048. In yet some other examples, an antigen binding domain against EphA2 is an antigen binding portion, e.g., CDRs, of an antibody described in, e.g., Yu et al., Mol Ther 22 (1): 102-111 (2014). In yet some other examples, an antigen binding domain against GD3 is an antigen binding portion, e.g., CDRs, of an antibody described in, e.g., U.S. Pat. Nos. 7,253,263; 8,207,308; US20120276046; EP1013761 A3; 20120276046; WO2005035577; or U.S. Pat. No. 6,437,098. In yet some other examples, an antigen binding domain against fucosyl GM1 is an antigen binding portion, e.g., CDRs, of an antibody described in, e.g., US20100297138; or WO2007/067992. In yet some other examples, an antigen binding domain against sLe is an antigen binding portion, e.g., CDRs, of the antibody G193 (for lewis Y), see Scott A M et al, Cancer Res 60:3254-61 (2000), also as described in Neeson et al, J Immunol May 2013 190 (Meeting Abstract Supplement) 177.10. In yet some other examples, an antigen binding domain against GM3 is an antigen binding portion, e.g., CDRs, of the antibody CA 2523449 (mAb 14F7). In yet some other examples, an antigen binding domain against HMWMAA is an antigen binding portion, e.g., CDRs, of an antibody described in, e.g., Kmiecik et al., Oncoimmunology 3 (1): e27185 (2014)(PMID: 24575382)(mAb9.2.27); U.S. Pat. No. 6,528,481; WO2010033866; or US20140004124. In yet some other examples, an antigen binding domain against o-acetyl-GD2 is an antigen binding portion, e.g., CDRs, of the antibody 8B6. In yet some other examples, an antigen binding domain against TEM1/CD248 is an antigen binding portion, e.g., CDRs, of an antibody described in, e.g., Marty et al., Cancer Lett 235 (2): 298-308 (2006); Zhao et al., J Immunol Methods 363 (2): 221-232 (2011). In yet some other examples, an antigen binding domain against CLDN6 is an antigen binding portion, e.g., CDRs, of the antibody IMAB027 (Ganymed Pharmaceuticals), see e.g., clinicaltrial.gov/show/NCT02054351. In yet some other examples, an antigen binding domain against TSHR is an antigen binding portion, e.g., CDRs, of an antibody described in, e.g., U.S. Pat. Nos. 8,603,466; 8,501,415; or U.S. Pat. No. 8,309,693. In yet some other examples, an antigen binding domain against GPRC5D is an antigen binding portion, e.g., CDRs, of the antibody FAB6300A (R&D Systems); or LS-A4180 (Lifespan Biosciences). In yet some other examples, an antigen binding domain against CD97 is an antigen binding portion, e.g., CDRs, of an antibody described in, e.g., U.S. Pat. No. 6,846,911; de Groot et al., J Immunol 183 (6): 4127-4134 (2009); or an antibody from R&D: MAB3734. In yet some other examples, an antigen binding domain against ALK is an antigen binding portion, e.g., CDRs, of an antibody described in, e.g., Mino-Kenudson et al., Clin Cancer Res 16 (5): 1561-1571 (2010). In yet some other examples, an antigen binding domain against polysialic acid is an antigen binding portion, e.g., CDRs, of an antibody described in, e.g., Nagae et al., J Biol Chem 288 (47): 33784-33796 (2013). In yet some other examples, an antigen binding domain against PLAC1 is an antigen binding portion, e.g., CDRs, of an antibody described in, e.g., Ghods et al., Biotechnol Appl Biochem 2013 doi: 10.1002/bab.1177. In yet some other examples, an antigen binding domain against GloboH is an antigen binding portion of the antibody VK9; or an antibody described in, e.g., Kudryashov V et al, Glycoconj J.15 (3): 243-9 (1998), Lou et al., Proc Natl Acad Sci USA 111 (7): 2482-2487 (2014); MBr1: Bremer E-G et al. J Biol Chem 259:14773-14777 (1984). In yet some other examples, an antigen binding domain against NY-BR-1 is an antigen binding portion, e.g., CDRs of an antibody described in, e.g., Jager et al., Appl Immunohistochem Mol Morphol 15 (1): 77-83 (2007). In yet some other examples, an antigen binding domain against WT-1 is an antigen binding portion, e.g., CDRs, of an antibody described in, e.g., Dao et al., Sci Transl Med 5 (176): 176ra33 (2013); or WO2012/135854. In yet some other examples, an antigen binding domain against MAGE-A1 is an antigen binding portion, e.g., CDRs, of an antibody described in, e.g., Willemsen et al., J Immunol 174 (12): 7853-7858 (2005)(TCR-like scFv). In yet some other examples, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29 (4): 2923-2931 (2012). In yet some other examples, an antigen binding domain against Tie 2 is an antigen binding portion, e.g., CDRs, of the antibody AB33 (Cell Signaling Technology). In one embodiment, an antigen binding domain against MAD-CT-2 is an antigen binding portion, e.g., CDRs, of an antibody described in, e.g., PMID: 2450952; U.S. Pat. No. 7,635,753. In yet some other examples, an antigen binding domain against Fos-related antigen 1 is an antigen binding portion, e.g., CDRs, of the antibody 12F9 (Novus Biologicals). In yet some other examples, an antigen binding domain against MelanA/MART1 is an antigen binding portion, e.g., CDRs, of an antibody described in, EP2514766 A2; or U.S. Pat. No. 7,749,719. In yet some other examples, an antigen binding domain against sarcoma translocation breakpoints is an antigen binding portion, e.g., CDRs, of an antibody described in, e.g., Luo et al, EMBO Mol. Med. 4 (6): 453-461 (2012). In yet some other examples, an antigen binding domain against TRP-2 is an antigen binding portion, e.g., CDRs, of an antibody described in, e.g., Wang et al, J Exp Med. 184 (6): 2207-16 (1996). In yet some other examples, an antigen binding domain against CYP1B1 is an antigen binding portion, e.g., CDRs, of an antibody described in, e.g., Maecker et al, Blood 102 (9): 3287-3294 (2003). In one embodiment, an antigen binding domain against RAGE-1 is an antigen binding portion, e.g., CDRs, of the antibody MAB5328 (EMD Millipore). In yet some other examples, an antigen binding domain against human telomerase reverse transcriptase is an antigen binding portion, e.g., CDRs, of the antibody cat no: LS-B95-100 (Lifespan Biosciences). In yet some other examples, an antigen binding domain against intestinal carboxyl esterase is an antigen binding portion, e.g., CDRs, of the antibody 4F12: cat no: LS-B6190-50 (Lifespan Biosciences). In yet some other examples, an antigen binding domain against mut hsp70-2 is an antigen binding portion, e.g., CDRs, of the antibody Lifespan Biosciences: monoclonal: cat no: LS-C₁₃₃₂₆₁₋₁₀₀ (Lifespan Biosciences). In yet some other examples, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich. In yet some other examples, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114 (13): 2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56th ASH Annual Meeting and Exposition, San Francisco, Calif. Dec. 6-9, 2014. In yet some other examples, an antigen binding domain against CD72 is an antigen binding portion, e.g., CDRs, of the antibody J3-109 described in Leuk Lymphoma. 1995 June; 18 (1-2): 119-22; Cancer Res Mar. 15, 2009 69; 2358. In yet some other examples, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In yet some other examples, an antigen binding domain against FCAR is an antigen binding portion, e.g., CDRs, of the antibody CD89/FCAR Antibody (Catalog #10414-H08H), available from Sino Biological Inc. In yet some other examples, an antigen binding domain against LILRA2 is an antigen binding portion, e.g., CDRs, of the antibody LILRA2 monoclonal antibody (M17), clone 3C₇, available from Abnova, or Mouse Anti-LILRA2 antibody, Monoclonal (2D7), available from Lifespan Biosciences. In yet some other examples, an antigen binding domain against CD300LF is an antigen binding portion, e.g., CDRs, of the antibody Mouse Anti-CMRF35-like molecule 1 antibody, Monoclonal [UP-D2], available from BioLegend, or Rat Anti-CMRF35-like molecule 1 antibody, Monoclonal [234903], available from R&D Systems. In yet some other examples, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1.times.CD3 BiTE Antibody” 53rd ASH Annual Meeting and Exposition, Dec. 10-13, 2011, and MCLA-117 (Merus). In yet some other examples, an antigen binding domain against BST2 (also called CD317) is an antigen binding portion, e.g., CDRs, of the antibody Mouse Anti-CD317 antibody, Monoclonal [3H4], available from Antibodies-Online or Mouse Anti-CD317 antibody, Monoclonal [696739], available from R&D Systems. In yet some other examples, an antigen binding domain against EMR2 (also called CD312) is an antigen binding portion, e.g., CDRs, of the antibody Mouse Anti-CD312 antibody, Monoclonal [LS-B8033] available from Lifespan Biosciences, or Mouse Anti-CD312 antibody, Monoclonal [494025] available from R&D Systems. In yet some other examples, an antigen binding domain against LY75 is an antigen binding portion, e.g., CDRs, of the antibody Mouse Anti-Lymphocyte antigen 75 antibody, Monoclonal [HD30] available from EMD Millipore or Mouse Anti-Lymphocyte antigen 75 antibody, Monoclonal [A15797] available from Life Technologies. In still yet some other examples, an antigen binding domain against GPC3 is an antigen binding portion, e.g., CDRs, of the antibody hGC33 described in Anticancer Drugs. 2010 November; 21 (10): 907-916, or MDX-1414, HN3, or YP7, all three of which are described in FEBS Lett. 2014 Jan. 21; 588 (2): 377-82. In still yet some other examples, an antigen binding domain against FCRL5 is an antigen binding portion, e.g., CDRs, of the anti-FcRL5 antibody described in Mol Cancer Ther. 2012 October; 11 (10): 2222-32. In still yet some other examples, an antigen binding domain against IGLL1 is an antigen binding portion, e.g., CDRs, of the antibody Mouse Anti-Immunoglobulin lambda-like polypeptide 1 antibody, Monoclonal [AT1G4] available from Lifespan Biosciences, Mouse Anti-Immunoglobulin lambda-like polypeptide 1 antibody, Monoclonal [HSL11] available from BioLegendSad.

In still yet some other examples, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

The antigen binding domain can be any domain that binds to the antigen including but not limited to a monoclonal antibody, a polyclonal antibody, a recombinant antibody, a human antibody, a humanized antibody, and a functional fragment thereof, including a Fab, a Fab′, a F(ab′)₂, an Fv, a single chain antibody (e.g., scFv), a minibody, a diabody, a single-domain antibody (“sdAb” or “nanobodies” or “camelids”), or an Fc binding domain. In some instances, it may be beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some instances, the antigen binding domain are “cross-species” in that it binds to the counterpart antigen in a non-human primate, such as Callithrix jacchus, Saguinus oedipus or Saimiri sciureus, in order to facilitate a testing of immunogenicity of the antigen binding domain in these animals.

### Cytoplastic Domain of TFP or CAR

The cytoplasmic domain of the TFP or CAR can include an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in some cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal. Examples of intracellular signaling domains for use in the TFP or CAR of the disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. Examples of ITAM containing primary intracellular signaling domains that are of particular use in the disclosure include those of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, a CAR of the disclosure comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP or CAR can comprise the CD3-zeta signaling domain by itself or it can be combined with any other desired intracellular signaling domain(s) useful in the context of a CAR of the disclosure. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119 (3): 696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

The intracellular signaling sequences within the cytoplasmic portion of the TFP or CAR of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB.

### Transmembrane Domain of TFP or CAR

The extracellular region of TFP or CAR comprising an antigen binding domain can be linked to the intracellular region, for example by a transmembrane domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In one aspect, the transmembrane domain is one that is associated with one of the other domains of the TFP or CAR used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T-cell surface (or another CAR on the CAR-T cell surface). In a different aspect the amino acid sequence of the transmembrane domain may be modified or substituted so as to minimize interactions with the binding domains of the native binding partner present in the same TFP or CAR.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP or CAR has bound to a target. A transmembrane domain of particular use in this disclosure may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Where desired, a hinge sequence or linker can be utilized to connect the extracellular domain to the transmembrane domain. Nonlimiting examples of hinge sequences are hinge sequences derived from a human immunoglobulin (Ig) hinge, e.g., an IgG4 hinge, or a CD8a hinge. A variety of linkers, such as oligo- or polypeptide linkers, are available in the art for linking various domains together. They may vary in length from about 2 to 50 amino acids and vary in amino acid composition. A commonly utilized linker is one enriched in glycine, e.g., amino acid sequence of GGGGSGGGGS, or variations thereof.

In some embodiments, the TFP- or the CAR-expressing cell described herein can further comprise multiple types of TFPs or CARs capable of binding to different antigens, or different epitopes on the same antigen. For instance, a TFP- or CAR-expressing cell of the present disclosure can comprise a second TFP or CAR that includes a different antigen binding domain, e.g., to the same target (CD19 or BCMA) or a different target (e.g., CD123). In one embodiment, when the TFP-expressing cell comprises two or more different TFPs or CARs, the antigen binding domains of the different TFPs or CARs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a VHH. Similarly, a cell expressing a first and second CAR can have an antigen binding domain of the first CAR, e.g., as a fragment, e.g., a scFv, that does not form an association with the antigen binding domain of the second CAR, e.g., the antigen binding domain of the second CAR is a VHH.

In some other embodiments, the TFP- or CAR-expressing cell described herein can further express another agent, e.g., an agent which enhances the activity of a TFP- or CAR-expressing cell. For example, in one embodiment, the agent can be an agent which inhibits an inhibitory molecule. Inhibitory molecules, e.g., PD1, can, in some embodiments, decrease the ability of a TFP- or CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4, CD93, OX40, Siglec-15, and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T-cells and myeloid cells (Agata et al. 1996 Int. Immunol 8:765-75). Two ligands for PD1, PD-L1 and PD-L2 have been shown to downregulate T-cell activation upon binding to PD1 (Freeman et al. 2000 J Exp Med 192:1027-34; Latchman et al. 2001 Nat Immunol 2:261-8; Carter et al. 2002 Eur J Immunol 32:634-43). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-CD19 TFP described herein, improves the persistence of the T-cell. In one embodiment, the TFP or CAR comprises the extracellular domain of PD1. Alternatively, provided are TFPs or CARs containing an antibody or antibody fragment such as a scFv that specifically binds to the Programmed Death-Ligand 1 (PD-L1) or Programmed Death-Ligand 2 (PD-L2).

In some embodiments, the present disclosure provides a population or a mixture of populations of TFP- or CAR-expressing cells, in which PTPN2 expression or activity is downregulated (e.g., inhibited). In some examples, the population of TFP-expressing T-cells comprises a mixture of cells expressing different TFPs. The population of TFP-T-cells can include a first cell expressing a TFP having an anti-CD19 or anti-BCMA binding domain described herein, and a second cell expressing a TFP having a different anti-CD19 or anti-BCMA binding domain, e.g., an anti-CD19 or anti-BCMA binding domain described herein that differs from the anti-CD19 binding domain in the TFP expressed by the first cell. As another example, the population of TFP-expressing cells can include a first cell expressing a TFP that includes an anti-CD19 or anti-BCMA binding domain, e.g., as described herein, and a second cell expressing a TFP that includes an antigen binding domain to a target other than CD19 or BCMA (e.g., another tumor-associated antigen). The same approach may apply to a mixture of CAR-expressing cells, individual cells may target the same or different antigens.

Encompassed herein are also additional TFP or CAR configurations known in the art, including Split CARs, RCARs, as well as other TFP and CAR combinations described in WO2016187349, U.S. Pat. No. 9,856,497, WO2017123556, all of which are incorporated herein by reference in their entirety.

Further contemplated are allogeneic CAR-expressing cells in which expression or activity of PTPN2 is inhibited. For example, the cell can be an allogeneic T cell, e.g., an allogeneic T cell lacking expression of a functional T cell receptor (TCR) and/or human leukocyte antigen (HLA), e.g., HLA class I and/or HLA class II. In particular, a T cell lacking a functional TCR can be, e.g., engineered such that it does not express any functional TCR on its surface, or engineered such that it does not express one or more subunits that comprise a functional TCR, or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not substantially elicit an adverse immune reaction in a host.

Allogeneic T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, a CRISPR system, transcription activator like effector nuclease (TALEN), or zinc finger endonuclease (ZFN).

In some embodiments, an allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a TFP- or CAR-expressing cell to mount an immune effector response. Examples of inhibitory molecules include PD1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4 and TGF beta.

The nucleic acid sequences coding for a desired TFP or CAR can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. Where desired, the TFP- and CAR-expressing cells of the present disclosure are generated using lentiviral viral vectors.

Conventional viral and non-viral based gene transfer methods can be used to introduce TFP- or CAR-encoding sequences to a cell of interest, e.g., a lymphoid cell as disclosed herein. Such methods can be used to introduce the TFP- or CAR-encoding sequences to cells in culture, which in turn is administered into a subject. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted sequence. High transduction efficiencies can be observed in many different cell types and target tissues.

A subject lymphoid cell in which its PTPN2 expression and/or activity is downregulated (e.g., inhibited) finds an array of utility in treating a range of diseases associated with the antigen to which the TFP or CAR binds. For instance, PTPN2 downregulation (e.g., inhibition) enhances lymphoid cell expansion, effector function, and survival of human TFP- or CAR-expressing T cells in vitro, and human T cell persistence and antitumor activity in vivo.

In one aspect, the present disclosure provides a method of augmenting activity of an effector cell (e.g., T cells, NK cells, KHYG cells). The method typically comprises: contacting said effector cell with an effective amount of a PTPN2 inhibitor, such as a compound of Formula (I), such that PTPN2 expression and activity is downregulated (e.g., inhibited) in said effector cell. Augmentation of effector activity can be evidenced by the cytolytic activity against a target cell such as a tumor or cancer cell, or helper activity including the release of cytokines. Assessing augmented effector function can be carried out using any methods known in the art or disclosed here. In some instances, cytotoxicity of an effector cell expressing TFP or CAR as disclosed herein can be greater in response to PTPN2-inhibitor treatment as compared to a control lymphoid cell lacking such treatment. A TFP- or CAR-expressing effector cell treated with a PTPN2 inhibitor can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to an effector cell lacking the treatment. In some embodiments, a change in cytotoxicity can comprise comparing such activity before and after treating the effector cell with a PTPN2 inhibitor. In some other instances, release of cytotoxic cytokines of an effector cell expressing TFP or CAR as disclosed herein can be greater in response to treating with a PTPN2 inhibitor as compared to a control lymphoid cell lacking such treatment. Exemplary cytokines include IFNγ, TNFα, CSF, TGFB, IL-1, IL-2, IL-4, IL-5, IL-6, IL-13, IL-17, IL-21, IL-22, granzyme, and the like. A TFP- or CAR-expressing effector cell can generate about 1 fold, 2 fold, 3 fold, 4 fold, 5 fold, 6 fold, 7 fold, 8 fold, 9 fold, 10 fold, 50 fold, 100 fold or greater release of cytotoxic cytokines in response to a PTPN2 inhibitor treatment as compared to a control lymphoid cell that is not being exposed to the PTPN2 inhibitor.

In another aspect, the present disclosure provides a method of treating cancer in a subject in need thereof, comprising: administering to the subject an effective amount of lymphoid cells, wherein an individual lymphoid cell comprises (a) a chimeric T-cell receptor sequence encoding a T-cell receptor fusion protein (TFP), and/or (b) a chimeric antigen receptor (CAR) sequence encoding a CAR, wherein each of TFP and CAR, when present, exhibits specific binding to an antigen, and wherein expression and/or function of PTPN2 in said cell is downregulated (e.g., inhibited). In some embodiments of the disclosure, downregulation of PTPN2 expression and/or activity can be affected by one or more types of PTPN2 inhibitor disclosed herein. Where desired, downregulation of expression or activity of PTPN2 takes place transiently by contacting the cells with a small molecule PTPN2 inhibitor, such as a compound of Formula (I), or a nucleic acid based PTPN2 inhibitor (e.g., siRNA or shRNA) that asserts such downregulation transiently without being integrated into the cell's genome. Alternatively, PTPN2 downregulation can occur permanently by contacting the cell with a PTPN2 inhibitor that disrupts the expression of the PTPN2 gene permanently by cleaving such gene with a CRISPR-based PTPN2 inhibitor.

In some examples, the practice of the subject method involves downregulating PTPN2 expression and/or activity in the lymphoid cells, ex vivo, prior to administering an effective amount of PTPN2-treated lymphoid cells (e.g., effector cells) to the subject. The ex vivo inhibition can be carried out prior to, concurrent with, or after the introduction of the nucleic acid encoding the TFP or CAR into the lymphoid cell. Such ex vivo treatment may facilitate the expansion and proliferation of the effector cells to yield to a cell count reaching a desired effective amount to be administered to a subject. Such ex vivo treatment may also prolong the survival effector cell persistence and antitumor activity in vivo. For instances, an effector cell of the present disclosure when infused into a subject is capable of killing tumor or cancer cells in the subject. Unlike antibody therapies, TFP-modified or CAR-modified immune effector cells (e.g., T cells, NK cells, KHYG cells) are able to replicate in vivo resulting in long-term persistence that can lead to sustained tumor control. In various aspects, the immune effector cells (e.g., T cells, NK cells, KHYG cells) administered to the subject, or their progeny, persist in the subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell or KHYG cells to the subject.

Accordingly, the present disclosure also provides a method of increasing the therapeutic efficacy of a TFP- or CAR-expressing cell directed to a tumor or tumor associated antigen. In some embodiments, administering a PTPN2 inhibitor occurs ex vivo. In other embodiments, administering a PTPN2 inhibitor occurs in vivo prior to, concurrent with, or after the cells have been administered to a subject, where the cell may have or may not have previously been exposed to the PTPN2 inhibitor ex vivo.

In one aspect, a fully-human TFP- or CAR-modified immune effector cells (e.g., T cells, NK cells, KHGY cells) of the disclosure may be a type of vaccine for ex vivo immunization and/or in vivo therapy in a mammal including a human.

The subject methods utilizing a TFP- or CAR-expressing lymphoid cells (including e.g., effector cells) that target one or more tumor antigens can be applied to treat solid tumor and hematological cancers. For example, the subject methods can be used to treat: Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Basal Cell Carcinoma of the Skin, Bile Duct Cancer, B1adder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma-see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sézary Syndrome), Ductal Carcinoma In Situ (DCIS), Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma (Soft Tissue Sarcoma), Kidney (Renal Cell) Cancer, Laryngeal Cancer (Head and Neck Cancer), Leukemia, Lip and Oral Cavity Cancer (Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma (Skin Cancer), Mesothelioma, Malignant, Metastatic Cancer, Metastatic Squamous Neck Cancer with Occult Primary (Head and Neck Cancer), Midline Tract Carcinoma, Mouth Cancer (Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary B1astoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell)

Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and any of the aforementioned cancers exhibiting expression and/or activity of PTPN2 in the cancer cells.

The present disclosure also provides pharmaceutical compositions comprising a TFP- or CAR-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity of administration will be determined by such factors as the condition of the subject, and the type and severity of the subject's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The precise effective amount of the compositions of the present disclosure to be administered can be determined by a physician with consideration of individual differences in age, weight, tumor size, extent of infection or metastasis, and condition of the subject. It can generally be stated that a pharmaceutical composition comprising the T-cells described herein may be administered at a dosage of 10⁴ to 10⁹ cells/kg body weight, in some instances 10⁵ to 10⁶ cells/kg body weight, including all integer values within those ranges. T-cell compositions may also be administered multiple times at these dosages. The cells can be administered by using infusion techniques that are commonly known in immunotherapy (see, e.g., Rosenberg et al., New Eng. J. of Med. 319:1676, 1988).

In some examples, it may be desired to administer activated T-cells to a subject and then subsequently redraw blood (or have an apheresis performed), activate T-cells therefrom according to the present disclosure, and reinfuse the patient with these activated and expanded T-cells. This process can be carried out multiple times every few weeks. In certain aspects, T-cells can be activated from blood draws of from 10 cc to 400 cc. In certain aspects, T-cells are activated from blood draws of 20 cc, 30 cc, 40 cc, 50 cc, 60 cc, 70 cc, 80 cc, 90 cc, or 100 cc. In some embodiments, the T-cell compositions of the present disclosure are administered by i.v. injection. The compositions of T-cells may be injected directly into a tumor, lymph node, or site of infection.

In some examples, a subject may undergo leukapheresis, wherein leukocytes are collected, enriched, or depleted ex vivo to select and/or isolate the cells of interest, e.g., T-cells. These T-cell isolates may be expanded by methods known in the art and treated such that one or more TFP constructs of the disclosure may be introduced, thereby creating a TFP-expressing or CAR-expressing T-cell of the disclosure.

### Pharmaceutical Compositions and Methods of Administration

In an aspect is provided a pharmaceutical composition comprising a compound described herein, or a pharmaceutically acceptable salt or solvate thereof, and a pharmaceutically acceptable excipient.

In some embodiments, a compound described herein, or a pharmaceutically acceptable salt or solvate thereof, is administered to a subject in a biologically compatible form suitable for administration to treat or prevent diseases, disorders, or conditions. Administration of a compound described herein can be in any pharmacological form including a therapeutically effective amount of a compound described herein, or a pharmaceutically acceptable salt or solvate thereof, alone or in combination with a pharmaceutically acceptable carrier.

In some embodiments, a compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s)(or carrier(s)) is acceptable or suitable if the excipient is compatible with the other ingredients of the composition and not deleterious to the recipient (i.e., the subject) of the composition.

In some embodiments of the methods described herein, a compound described herein is administered either alone or in combination with pharmaceutically acceptable carriers, excipients or diluents, in a pharmaceutical composition. Administration of a compound or composition described herein can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, a compound described herein can be administered locally to the area in need of treatment, by, for example, local infusion during surgery, topical application such as creams or ointments, injection, catheter, or implant. The administration can also be by direct injection at the site of a diseased tissue or organ. In some embodiments, a compound described herein, or a pharmaceutically acceptable salt or solvate thereof, is administered orally.

In some embodiments of the methods described herein, a pharmaceutical composition suitable for oral administration is presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary, or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compound which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided for illustrative purposes only and not to limit the scope of the claims provided herein. Unless noted otherwise, all materials, such as reagents, starting materials and solvents, were purchased from commercial suppliers, such as Sigma-Aldrich, VWR, and the like, and were used without further purification. Reactions were run under nitrogen atmosphere, unless noted otherwise. The progress of reactions was monitored by thin layer chromatography (TLC), analytical high performance liquid chromatography (anal. HPLC), and mass spectrometry, the details of which may be provided in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, for example, using Microsorb C18 or Microsorb BDS column packings and conventional eluents. Progress of reactions was typically monitored by liquid chromatography mass spectrometry (LCMS). Characterization of isomers was typically done by Nuclear Overhauser effect spectroscopy (NOE). Characterization of reaction products was routinely carried out by mass spectrometry and/or ¹H-NMR spectroscopy. For NMR measurement, samples were dissolved in deuterated solvent (CD₃OD, CDCl₃, or DMSO-d₆).

### Example 1a: Synthesis of 5-(8-fluoro-5-hydroxy-2-methoxyquinazolin-7-yl)—1,2,5-thiadiazolidin-3-one 1,1-dioxide (Compound 101)

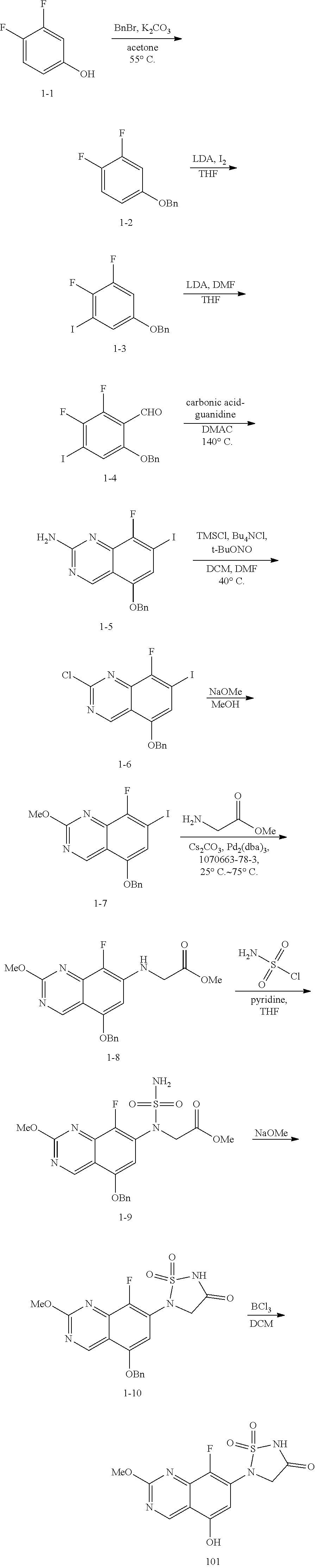

Step A: To a solution of 1-1 (100 g, 768 mmol, 1.0 eq) in acetone (800 mL) were added K₂CO₃ (265.6 g, 1.92 mol, 2.5 eq) and BnBr (144.6 g, 845 mmol, 100 mL, 1.1 eq) at 25° C. After addition, the mixture was stirred at 55° C. for 5 h. The mixture was diluted with H₂O (1200 mL) and extracted with MTBE (600 mL×2). The organic layers were combined and washed with brine (600 mL), then concentrated under reduced pressure to give a residue, which was purified by silica gel chromatography with petroleum ether:ethyl acetate (1: 0˜50:1) to give 1-2 (140 g) as a white solid. ¹HNMR (400 MHZ, CDCl₃): δ 7.44-7.34 (m, 5H), 7.11-7.04 (m, 1H), 6.84-6.78 (m, 1H), 6.71-6.66 (m, 1H), 5.03 (s, 2H).

Step B: To a solution of 1-2 (140 g, 635 mmol, 1.0 eq) in THF (1200 mL) was added LDA (2 M, 381 mL, 1.2 eq) dropwise at −70° C. The mixture was stirred at −70° C. for 0.5 h, then a solution of I2 (242 g, 953 mmol, 1.5 eq) in THF (200 mL) was added at −70 ˜-60° C. and the resulting mixture stirred an additional 2 h. The reaction mixture was quenched with saturated NH₄Cl solution (1500 mL) and EtOAc (800 mL×2). The organic layers were combined and washed with brine (800 mL). The organic layer was concentrated under reduced pressure to give the crude product, which was purified by silica gel chromatography with petroleum ether: ethyl acetate (1: 0˜50:1) to give 1-3 (166 g) as a white solid. ¹HNMR (400 MHz, CDCl₃): δ 7.50-7.48 (m, 2H), 7.44-7.40 (m, 1H), 7.37-7.34 (m, 1H), 7.15-7.07 (m, 1H), 6.62-6.58 (m, 1H), 5.14 (s, 2H).

Step C: To a solution of 1-3 (166 g, 479 mmol, 1.0 eq) in THF (1600 mL) was added LDA (2 M, 288 mL, 1.2 eq) dropwise at −70° C. The mixture was stirred for 0.5 h, then DMF (52.6 g, 719 mmol, 55.4 mL, 1.5 eq) was added at −70 ˜-60° C. and the resulting mixture stirred for 1 h. The reaction mixture was quenched with saturated NH₄Cl solution (1500 mL) and EtOAc (800 mL×2). The organic layers were combined and washed with brine (800 mL). The organic layer was concentrated under reduced pressure to give the crude product, which was purified by silica gel chromatography with petroleum ether:ethyl acetate (1: 0˜30:1) to give 1-4 (79.0 g) as an off-white solid. ¹HNMR (400 MHz, DMSO-d₆): δ 10.25 (s, 1H), 7.64-7.63 (m, 1H), 7.50-7.34 (m, 5H), 5.28 (s, 2H).

Step D: To a mixture of 1-4 (60.0 g, 160 mmol, 1.0 eq) in DMAC(480 mL) was added carbonic acid: guanidine (37.6 g, 208 mmol, 1.3 eq). The mixture was stirred at 140° C. for 12 h, then cooled to room temperature and poured into H₂O (1200 mL). The mixture was filtered to give a filter cake, which was concentrated under reduced pressure to give 1-5 (52.0 g) as a gray solid, which was used in next step without further purification. ¹HNMR (400 MHZ, DMSO-d₆): δ 9.22 (s, 1H), 7.54-7.52 (m, 2H), 7.44-7.31 (m, 5H), 7.05 (s, 1H), 5.26 (s, 2H).

Step E: To a stirred solution of 1-5 (26.0 g, 65.8 mmol, 1.0 eq) in DCM (390 mL) and DMF (39 mL) were added TMSCI (28.6 g, 263 mmol, 33.4 mL, 4.0 eq) and BuNCI (21.9 g, 78.9 mmol, 1.2 eq). t-BuONO (37.3 g, 361 mmol, 43.0 mL, 5.5 eq) was added to the mixture at 0° C. The reaction mixture was stirred at 40° C. for 3 h, then quenched with aqueous NH₄Cl (300 mL) and extracted with DCM (200 mL×2). The combined organic phases were washed with brine (200 mL), dried over Na₂SO₄ and concentrated under reduced pressure to give a residue, which was purified by chromatography on a silica gel column with petroleum ether:ethyl acetate (100: 1˜1:1) to give 1-6 (11.0 g) as a yellow solid. ¹HNMR (400 MHZ, DMSO-d₆): δ 9.62 (s, 1H), 7.66-7.65 (m, 1H), 7.58-7.56 (m, 2H), 7.46-7.36 (m, 3H), 5.39 (s, 2H).

Step F: To a solution of 1-6 (6.50 g, 15.7 mmol, 1.0 eq) in MeOH (65 mL) was added NaOMe (4.32 g, 79.9 mmol, 5.1 eq). The mixture was stirred at 70° C. for 4 h, then cooled to room temperature and filtered to give a filter cake. The filter cake was triturated with H₂O (15 mL) for 0.5 h. The mixture was filtered to give a filter cake, which was dried to give 1-7 (5.00 g) as a yellow solid. The product was used in the next step without further purification. ¹HNMR (400 MHZ, DMSO-d₆): δ 9.50 (s, 1H), 7.56-7.55 (m, 2H), 7.45-7.35 (m, 4H), 5.33 (s, 2H), 4.03 (s, 3H).

Step G: Four reactions were carried out in parallel. To a solution of 1-7 (500 mg, 1.22 mmol, 1.0 eq) in dioxane (8 mL) was added methyl 2-aminoacetate hydrochloride (306.09 mg, 2.44 mmol, 1.3 eq). Cs2CO₃ (1.59 g, 4.88 mmol, 4.0 eq), dicyclohexyl-[3,6-dimethoxy-2-(2,4,6-triisopropylphenyl)phenyl] phosphane (130.86 mg, 0.243 mmol, 0.2 eq) and Pd2 (dba) 3 (223 mg, 0.243 mmol, 0.2 eq) were then added. The mixture was stirred at 75° C. for 5 h. The four reaction mixtures were cooled to room temperature, then diluted with H₂O (40 mL) and EtOAc (30 mL). The organic layer was separated and washed with brine (20 mL), then concentrated under reduced pressure to give a residue, which was purified by silica gel chromatography with petroleum ether:ethyl acetate (40: 1˜0:1) to give 1-8 (650 mg) as a yellow solid. LC-MS m/z: 372.0 [M+H]⁺.

Step H: To a mixture of 1-8 (500 mg, 1.35 mmol, 1.0 eq) in THF (10 mL) and pyridine (958 mg, 12.1 mmol, 9.0 eq) was added sulfamoyl chloride (778 mg, 6.73 mmol, 5.0 eq) at −5° C. After stirring for 1h, crude 1-9 in the reaction mixture was used in the next step without further work-up.

Step I: To a mixture of 1-9 was added NaOMe (1.45 g, 8.08 mmol, 30% purity, 6.0 eq) at −5° C. The reaction was stirred for 1 h, then concentrated under reduced pressure at 0-5° C. to give a residue. The crude product was purified by prep-HPLC to afford 1-10 (115 mg) as a yellow solid.

Step J: To a stirred solution of 1-10 (80 mg, 0.191 mmol, 1.0 eq) in DCM (1.6 mL) was added BC13 (1 M, 1.15 mL, 6.0 eq) at −40° C. The mixture was stirred for 4 h, then concentrated to give a residue. The crude product was dissolved with ACN(3 mL) and adjusted to pH 6˜7 using NH₃H₂O, then purified by prep-HPLC to give compound 101 (35 mg) as a yellow solid. LC-MS m/z: 329.0 [M+H]⁺; ¹HNMR (400 MHZ, CD₃OD): δ 9.29 (s, 1H), 7.26 (d, J═6.0 Hz, 1H), 4.60 (s, 2H), 4.07 (s, 3H).

### Example 1b: Synthesis of 5-(8-fluoro-5-hydroxy-2-(methylamino) quinazolin-7-yl)—1,2,5-thiadiazolidin-3-one 1,1-dioxide (Compound 102)

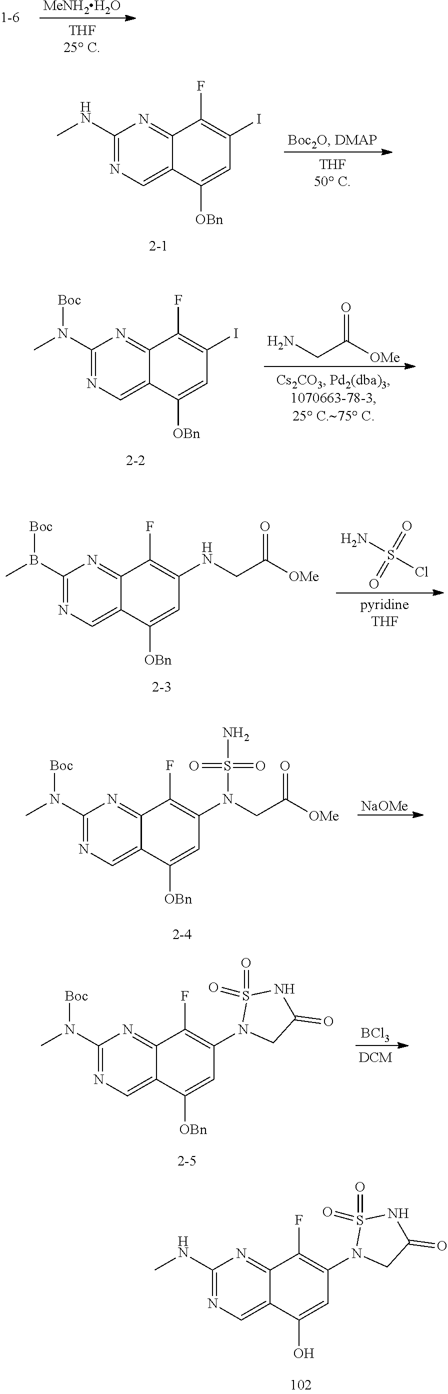

Step A: To a stirred solution of 1-6 (3.00 g, 7.24 mmol, 1.0 eq) in THF (24 mL) was added MeNH₂: H₂O (3.75 g, 36.18 mmol, 15 mL, 30% purity, 5.0 eq) at 0° C. The reaction mixture was stirred at 25° C. for 3 h, then quenched with H₂O (30 mL) and diluted with EtOAc (20 mL). The organic layer was separated, and the aqueous phase was extracted with EtOAc (10 mL). The organic layers were combined and washed with brine (20 mL), then concentrated under reduce pressure to give a residue, which was purified by chromatography on a silica gel column with petroleum ether:ethyl acetate (80: 1˜0:1) to give 2-1 (2.10 g) as a yellow solid. ¹HNMR (400 MHZ, DMSO-d₆): δ 9.18 (s, 1H), 7.82-7.75 (m, 1H), 7.54-7.34 (m, 5H), 7.07-6.90 (m, 1H), 5.26 (s, 2H), 2.89 (s, 3H).

Step B: To a stirred solution of 2-1 (2.00 g, 4.89 mmol, 1.0 eq) in THF (20 mL) were added DMAP (597 mg, 4.89 mmol, 1.0 eq) and Boc₂O (3.20 g, 14.7 mmol, 3.0 eq). The reaction mixture was stirred at 50° C. for 1.5 h, then cooled to room temperature and quenched with H₂O (20 mL) and diluted with EtOAc (20 mL). The organic layer was separated and washed with brine (20 mL), then concentrated under reduce pressure to give a residue, which was purified by chromatography on a silica gel column with petroleum ether:ethyl acetate (80: 1˜0:1) to give 2-2 (1.78 g) as a yellow solid. ¹HNMR (400 MHZ, DMSO-d₆): δ 9.57 (s, 1H), 7.57-7.55 (m, 2H), 7.46-7.37 (m, 4H), 5.35 (s, 2H), 3.39-3.32 (m, 3H), 1.46 (s, 9H).

Step C: Three reactions were carried out in parallel. To a solution of 2-2 (600 mg, 1.18 mmol, 1.0 eq) in dioxane (6 mL) was added methyl 2-aminoacetate hydrochloride (192 mg, 1.53 mmol, 1.3 eq). Cs2CO₃ (1.15 g, 3.53 mmol, 3.0 eq), dicyclohexyl-[3,6-dimethoxy-2-(2,4,6-triisopropylphenyl)phenyl] phosphane (126 mg, 0.235 mmol, 0.2 eq) and Pd2 (dba) 3 (216 mg, 0.235 mmol, 0.2 eq) were the added and the resulting mixture stirred at 75° C. for 5 h. The three reaction mixtures were cooled to room temperature, then diluted with H₂O (20 mL) and EtOAc (20 mL). The organic layer was separated and washed with brine (20 mL), then concentrated under reduced pressure to give a residue, which was purified by silica gel chromatography with petroleum ether: ethyl acetate (50: 1˜0:1) to give 2-3 (330 mg) as a yellow solid.

Step D: To a mixture of 2-3 (330 mg, 0.701 mmol, 1.0 eq) in THF (6.6 mL) and pyridine (499 mg, 6.31 mmol, 9.0 eq) was added sulfamoyl chloride (405 mg, 3.51 mmol, 5.0 eq) at −5° C. The reaction was stirred for 1 h, then the crude product (2-4) was used in the next step without further work-up.

Step E: To a mixture of 2-4 was added NaOMe (758 mg, 4.21 mmol, 30% purity, 6.0 eq) at −5° C. The reaction was stirred for 1 h, then concentrated under reduced pressure at 0˜5° C. to give a residue. The crude product was purified by prep-HPLC to give 2-5 (125 mg) as a yellow solid.

Step F: To a stirred solution of 2-5 (100 mg, 0.193 umol, 1.0 eq) in DCM (2 mL) was added BCl3 (1 M, 1.55 mL, 8.0 eq) at −40° C. The mixture was stirred at 20° C. for 2 h. The crude product was dissolved with ACN(3 mL) and the mixture was adjusted to pH 8-11 with NH₃H₂O. The crude product was purified by prep-HPLC to give compound 102 (22 mg) as a yellow solid. LC-MS m/z: 328.0 [M+H]⁺; ¹HNMR (400 MHZ, CD₃OD): δ 9.11 (s, 1H), 7.02 (d, J═5.6 Hz, 1H), 4.58 (s, 2H), 3.03 (s, 3H).

### Example 2: Phosphatase Activity Assay

### Assessing Selectivity and Potency of a Small Molecule PTPN2 Inhibitor

The selectivity and potency of a small molecule PTPN2 inhibitor as provided herein against one or more protein tyrosine phosphatase (PTP) enzymes, and specifically against PTPN2, is assessed in various ways. The one or more PTP enzymes comprise Mycobacterium protein tyrosine phosphatase A (mPTPA), Mycobacterium protein tyrosine phosphatase B (mPTPB), PTPN1 (i.e., PTP1B), PTPN2 (i.e., TC-PTP), PTPN22 (i.e., LYP), SHP-1, SHP-2, FAP-1, Meg2, HePTP, Laforin, VHX, VHR, LMWPTP, Cdc14A, LAR, CD45, PTPRG, a fragment thereof, a variant thereof, and a combination thereof. Selectivity and potency of a small molecule PTPN2 inhibitor is evaluated using a PTP activity inhibition assay. The assay is performed using a buffer comprising 50 mM HEPES buffer pH 7.5, 150 mM NaCl, 0.5 mM EDTA, 1 mM DTT, 0.001% Tween-20.

The assay is performed using a phosphated substrate, e.g., 10 mM 6,8-Difluoro-4-Methylumbelliferyl Phosphate (DIFMUP) that is stored at −20° C. Alternatively or in addition to, the assay is performed using other phosphated substrate (e.g., fluorescein diphosphate). Each PTP enzyme is diluted in the assay buffer

The assay can be carried out at room temperature in multiplate format, e.g., using 384 well plates. A small molecule PTPN2 inhibitor dissolved in DMSO at one of 10 concentrations from a serial dilution or DMSO alone for control is added to each well. A mixture of the assay buffer comprising PTP enzyme (e.g., 0.025 ng/μl PTPN2) is added to each well and mixed for approximately 2 minutes. The reaction is initiated by adding DiFMUP diluted in the assay buffer to a final concentration of approximately 45 μM DiFMUP in PTPN2 assay. For each PTP enzyme, DiFMUP is added at the Km (Michaelis constant) of the enzyme that had been independently determined. The phosphatase activity of the PTP enzyme is assessed by monitoring appearance of a fluorescent product (6,8-difluoro-7-hydroxyl-4-coumarin (DiFMU) from DiFMUP) continuously for about 15 to 30 minutes, using the INFINITE M1000Pro plate reader (Tecan) with excitation at 360 nm and emission at 450 nm (cutoff filter at 435 nm) for DiFMU. Each assay is performed at least in duplicate. The rate (e.g., the initial rate) of DiFMU formation is plotted against the concentration of the small molecule PTPN2 inhibitor, and the data is fitted (e.g., using a 4-parameter equation) to determine the inflection point of the fit as the IC₅₀ of the small molecule PTPN2 inhibitor for a specific enzyme.

### Example 3: Cytokine Release Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor for 12-24 hours (optionally longer). The activated effector cells are induced to release cytokine by co-culturing with target cells at a desired Effector: Target cell ratio, e.g., 10:1, 5:1, or 1:1. Co-culture supernatant is harvested after approximately 20 hrs. These supernatants are then used to measure the released cytokines such as IL-2 and IFN-g, using Meso Scale Discovery, Proinflammatory Panel 1 catalog #N05049A-1 system according to the manufacturer's protocol. The target cells can be irradiated (e.g. at 10,000) prior to co-culturing with the effector cells. This assay is to demonstrate that inhibiting PTPN2 by a small molecule PTPN2 inhibitor causes an increase in cytokine release (e.g., IL-2 or IFN-g) by T cells in response to the antigen to which the TFP or CAR binds.

### Example 4: Cell Proliferation Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor for 12-24 hours (optionally longer). The activated effector cells are induced to proliferate by co-culturing with target cells that comprise the target tumor antigen to which the TFP or CAR binds. Typically, the target cells are irradiated, washed and counted. Co-culturing is performed at a desired Effector: Target cell ratio, e.g., 10:1, 5:1, or 1:1. Proliferation of the effector cells are evaluated, typically after bead expansion for about 10 days. The number of cells per mL and the viability of cells are measured by Cellometer. This example is to demonstrate that PTPN2 inhibition by a small molecule PTPN2 inhibitor yields an increase in effector cell number and viability relative to effector cells not treated with a small molecule PTPN2 inhibitor.

### Example 5: Cytotoxicity Assay

Effector cells (e.g., non-modified T cells, or TFP- or CAR-expressing T cells) are activated with CD3/CD28 beads (CD3/CD28 CTS Dynabeads) in the presence of a PTPN2 inhibitor, such as a compound of Formula (I), for 12-24 hours (optionally longer). Target cells (e.g., cancer or tumor cells) that comprise the target tumor antigen to which the TFP or CAR binds are incubated with Calcein-AM in the dark, washed, and counted. The activated effector cells are co-cultured with the target cells. Co-culturing is performed at a desired Effector: Target cell ratio, e.g., 10:1, 5:1, or 1:1. At the end of the co-culture period (e.g., 5 hours), the number of target cells and the viability of the target cells are assessed by measuring Calcein fluorescence from the collected cells. This example is to demonstrate that PTPN2 inhibition by a PTPN2 inhibitor yields an increase in cytotoxicity of effector cells against target cells relative to effector cells not treated with a PTPN2 inhibitor.

### Example 6: CAR-T Killing Assay

The ability of PTPN2 inhibitors to potentiate tumor cell killing using CAR-Ts that express a tumor antigen (e.g., HER2) is demonstrated as follows. HER-2 specific CAR-T cells (CAR-Ts) are generated by transducing primary human CD3+ T cells

(Discovery Life Sciences) with a lentivirus expressing a chimeric antigen receptor specific to human HER2, as well as GFP (Creative Bio). Prior to transduction, T cells are stimulated overnight with anti-CD3 and anti-CD28 antibodies coated onto magnetic beads

(Invitrogen) at a 1:1 bead-to-cell ratio. Four days after transduction, the beads are removed and the following day CAR-Ts were sorted based on GFP expression and expanded in hIL7 (10 ng/ml)(Peprotech) and hIL15 (5 ng/ml)(Peprotech) for an additional 8 days. Thereupon, CAR-Ts are co-cultured with a Nuclight Red (Essen Biosciences)-labeled HER-2 positive tumor line (OVCAR-3) or a HER-2 negative line (HEK293T) for 18 h at a 1:1 effector: target cell ratio.

CAR-Ts are pretreated with 0.1% DMSO (vehicle control) or a PTPN2 inhibitor for 1-2.5 hours prior to co-culture with cell lines at indicated concentrations. PTPN2 inhibitor is washed from CAR-Ts prior to inclusion in the tumor killing assay. Tumor killing is assessed by comparing DMSO-treated CAR-Ts to PTPN2 inhibitor-treated CAR-Ts using either flow cytometry or Incucyte imaged-based assessment of tumor cell viability, at various effector: target ratios. Percent killing is assessed by calculating the number of viable cells at a given time point as compared to untreated tumor cells, tumor cells treated with PTPN2 inhibitor, and tumor cells cultured with DMSO treated CAR-T cells as a control. The results demonstrate that: (a) PTPN2 treated CAR-T cells exhibit a higher tumor cell killing activity as compared to control cells treated with DMSO; and (b) even a transient treatment of PTPN2 (e.g., for an hour followed by washing) is sufficient to potentiate the ability of CAR-T cells to kill tumor cells.

### Example 7: CAR-T Adoptive Cell Transfer Xenograft Tumor Assay

For in vivo studies with CAR-Ts, nude mice are implanted with OVCAR xenografts. After reaching a suitable size of 50-100 mm3, approximately 106 CAR-Ts transiently treated (e.g., for 1 hr and then washing away) with or without PTPN2 inhibitor are transferred i.v. into tumor bearing mice. Tumor volume and CAR-T cell count are measured at multiple time points and compared to control groups treated with DMSO (e.g., for 1 hr and then washing away). It is expected that mice administered with CAR-Ts that are treated with PTPN2 inhibitor exhibit lower tumor volume, higher frequency of CAR-Ts in the blood, and/or greater infiltration and/or activation of CAR-Ts into tumors, smaller tumor volume and/or higher CAR-T cell counts.

### Example 8: Mouse Adoptive Cell Transfer Syngeneic Tumor Assay

Thyl.1 congenic C₅₇BL/6 mice are implanted with approximately 5 ×10⁵ of OVA expressing syngeneic tumor cells (B16-OVA, ELA OVA, or YUMM1.1) formulated with 50% Matrigel (50% PBS). Prior to the implantation, the B16-OVA, ELA OVA, or YUMM1.1 tumor cell lines are transduced with a lentivirus encoding an OVA-GFP fusion protein. After sorting for GFP expression, the B16-OVA, EL4 OVA, or YUMM1.1 cells are shown to grow in untreated C₅₇BL/6 mice. After growing to a volume of ˜50-100 mm3, tumor bearing mice would receive i.v. transfer of approximately 1×10⁶ OT-1 transgenic T cells that will undergo the following treatments. First, primary OT-1 splenocytes are treated with 10 nm of SIINFEKL peptide or anti-CD3/anti-CD28 coated beads. After 2 days, the cells are washed and transferred into culture medium with IL-2, IL-7 and IL-15 (all at 5 ng/ml) for another 3 days. In other experiments, naïve OT-1 CD8 T cells are isolated for transfer. Prior to the transfer, the OT-1 cells are treated with DMSO (vehicle control) or a PTPN2 inhibitor for 1 hour and washed in PBS two times prior to injection.

The test groups are separated as follows: Tumor alone, Tumor+DMSO treated OT-1s, Tumor+PTPN2 inhibitor treated OT-1s. Each group may include 8 mice for the 2 time points tested. To assess in vivo efficacy, tumor volume is measured 3×/week using calipers at various time points post OT-1 injection. Furthermore, at day 7, the first group of mice are sacrificed to compare immune activation and infiltration in both secondary lymphoid tissue and in tumors, staining for markers including but not limited to CD4, CD8, CD25, CD69, CD44, CD62L, TCF1, TOX, TIM3, PD1. The abundance and activation state of immune cells are quantified using flow cytometry. The results are expected to demonstrate that a transient treatment with PTPN2 is sufficient to potentiate anti-tumor killing as evidenced by (a) a decrease in tumor volume, and/or (b) an increase in the abundance of activated T cells in spleen, lymph nodes and/or in tumor.

### Example 9: Mobility Shift Assay Used to Determine Potency of PTPN2 Inhibitors

Compound activity can be determined using PTPN2 in an in vitro enzymatic reaction. The enzymatic assay used to determine activity can be a mobility shift assay using a LabChip EZ Reader by Caliper Life Sciences. The enzymatic reaction is carried out in assay buffer (50 mM HEPES pH 7.5, 1 mM EGTA, 10 mM EDTA, 0.01% Tween® 20, and 2 mM DTT). The compounds are dispensed on a white 384 well ProxiPlate™ (PerkinElmer Catalog #6008289) plate using the Labcyte Echo at varying concentrations (12 point, 1:3 dilution). The enzyme (at 0.5 nM) is incubated with compound for 10 minutes at room temperature. Then the substrate (phosphorylated insulin receptor probe sequence is added at 2 μM to the plates and incubated for another 10 minutes at room temperature. Finally, a quench solution (water and 4-bromo-3-(2-oxo-2-propoxyethoxy)—5-(3-{[1-(phenylmethanesulfonyl) piperidin-4-yl]amino}phenyl)thiophene-2-carboxylic acid) is added to the plates, which are then run on the EZ Reader (excitation 488 nm, emission 530 nm) to measure % conversion (the amount of phosphorylated substrate which is de-phosphorylated by PTPN2). Each plate has a 100% control (inhibitor: 4-bromo-3-(2-oxo-2-propoxyethoxy)—5-(3-{[1-(phenylmethanesulfonyl) piperidin-4-yl] amino}phenyl)thiophene-2-carboxylic acid) and 0% control (DMSO), which are used to calculate % inhibition. The % inhibition is then used to calculate the IC₅₀ values.

### Example 10: Mobility Shift Assay (MSA) to Determine Potency of PTPN1 (Also Known as PTP1B) Inhibitors

Compound activity can be determined using full-length PTPN1 protein in an in vitro enzymatic reaction. The enzymatic assay to determine activity can be a mobility shift assay using a LabChip EZ Reader by Caliper Life Sciences. The enzymatic reaction is carried out in assay buffer (50 mM HEPES pH 7.5, 1 mM EGTA, 10 mM EDTA, 0.01% Tween® 20, and 2 mM DTT). The compounds are dispensed on a white 384 well ProxiPlate™ (PerkinElmer Cat #6008289) plate using a Labcyte Echo® liquid handler at varying concentrations (12 point, 1:3 dilution). The enzyme (at 0.5 nM) is incubated with compound for 10 minutes at room temperature. Then the substrate (phosphorylated insulin 30 receptor probe sequence is added at 2 μM to the plates and incubated for another 10 minutes at room temperature. Finally, a quench solution (water and 4-bromo-3-(2-oxo-2-propoxyethoxy)—5-(3-{[1-(phenylmethanesulfonyl) piperidin-4-yl]amino}phenyl)thiophene-2-carboxylic acid) is added to the plates, which are then run on the EZ Reader (excitation 488 nm, emission 530 nm) to measure % conversion (the amount of phosphorylated substrate which is de-phosphorylated by PTPN1). Each plate has a 100% control (inhibitor: 4-bromo-3-(2-oxo-2-propoxyethoxy)—5-(3-{[1-(phenylmethanesulfonyl) piperidin-4-yl]amino}phenyl)thiophene-2-carboxylic acid) and 0% control (DMSO), which are used to calculate % inhibition. The % inhibition is then used to calculate the IC₅₀ values.

### Example 11: B16F10 IFNγ-Induced MHC Upregulation and Cellular Growth Inhibition

B16F10 mouse melanoma cells (ATCC Cat #CRL-6475, Manassas, VA) are seeded at a density of 500 cells per well in a 384-well clear bottom plate in 25 μL total volume of DMEM+10% FBS. Cells are allowed to adhere overnight at 37° C.±5% CO₂. On the following day, 12.5 μL of mouse IFNγ is added to half of the plate at a concentration of 2 ng/ml for a final assay concentration of 0.5 ng/ml of IFNγ. Media only (12.5 μL of DMEM+10% FBS) is added to the remainder of the plate. Compounds resuspended in DMSO at 100 mM are diluted in DMSO ranging from 100 mM to 0.001 mM and DMSO only controls are included. The compound/DMSO dilutions are further diluted 1:250 in DMEM+10% FBS, and 12.5 μL of these dilutions are added in triplicates to cells with or without IFNγ. Final compound concentrations range from 100 μM to 0.001 UM with a final DMSO concentration of 0.1%. The outer 2-well perimeter of the plate is not used. The plate is loaded into an IncuCyte® S3 Live Cell Analysis System (Essen Bioscience-Sartorius, Ann Arbor, MI) maintained in a 37° C.±5% CO₂ incubator, allowed to equilibrate for 2 hours, and imaged every 6 hours for 5 days. Confluence over time for compound dilutions in the presence and absence of IFNγ is measured. Growth inhibition values are obtained when the “DMSO/no IFNγ” control reaches confluence >95%. At these time points, the percent growth inhibition of each compound at the indicated concentration is calculated relative to the “DMSO/with IFNγ” control. At these same time points, MHC upregulation is assessed by flow cytometry. Compounds inhibiting PTPN2 are expected to show an increase in MHC levels in a dose-dependent manner.

### Example 12: T Cell Activation and Function

Pan T cells are isolated from C57BL6 splenocytes. Isolated T cells (50,000 cells/well in a 96 well flat-bottom plate) are cultured in RPMI 1640 supplemented with 10% FBS, 50 nM 2-mercatoethanol, 100 U/mL penicillin, and 100 μg/mL streptomycin, and incubated with the indicated concentration of compound or DMSO in duplicates. After 1 hour, mouse T cell activator CD3/CD28 Dynabeads are added at a 1:5 beads to cells ratio to stimulate the T cells for 2 or 3 days as described below. Alternatively, antibodies to CD3 and CD28 are plate-bound for stimulation. T cells with or without compound are incubated in the absence of T cell activator beads (media only) as control. After 2 days of stimulation, activation status of T cells is assessed by flow cytometry. T cells are first subjected to Zombie Violet™ Fixable Viability dye for dead cell exclusion, washed and then stained with BUV805 labeled anti-CD8, APC-R⁷⁰⁰ labeled anti-CD25 and PE labeled anti-CD69 antibodies. After staining, cells are fixed with 2% paraformaldehyde and acquired on a BD LSRFortessa™ X-20 flow cytometer using BD FACSDiva™ software and data is analyzed. Dead cells are excluded and frequencies of activated CD8 T cells is reported as the frequency of CD25+ or CD69+ cells within the CD8+ population. The expression level of CD25 and CD69 indicates the activation status of cells on a per cell basis and is evaluated by the mean fluorescence intensities (MFI) of CD25 and CD69. After 3 days of stimulation, supernatants are collected and IFNγ and TNFa in supernatants are assessed using an MSD V-plex assay (Meso Scale Discovery, Rockville, MD).

### Example 13: In vivo efficacy of compounds in MC38 and B16F10 tumor models

### Tumor Cell Inoculation and Treatments.

Cells are grown to passage 3 in vitro. A total of 1×10⁵ viable MC-38 or B16F10 cells are inoculated subcutaneously into the right flank of female C₅₇Bl/6 mice (7-12 weeks old) on Day 0. The injection volume is 0.1 mL and is composed of a 1:1 mixture of S-MEM and Matrigel® (Corning, NY, USA). Tumors are size matched on Day 14 and the mice have a mean body weight of ˜21 g. The mean tumor volume (TV) at size match is approximately 116±8 mm3. Following size match, treatments are initiated on the same day. Dosing of mice is conducted on a daily or twice daily schedule.

Tumor volume is calculated three times weekly. Measurements of the length (L) and width (W) of the tumor are taken via electronic caliper and the volume is calculated according to the following equation: V═L×W^{2/2} using Study Director Version 3.1.399.22 (Studylog Systems, Inc, CA, USA). Mice are euthanized when tumor volume is ≤3000 mm³ or skin ulcerations occurred. Tumor growth inhibition (TGI) is calculated as TGI═1-(Mean TVTimepoint (Treatment)/Mean TVTimepoint (Vehicle)) for each timepoint that tumor volumes are measured. Reported TGIMax is the largest TGI value for any timepoint that tumors volumes are collected for that treatment group. Where desired, at approximately day 7, a first group of mice are sacrificed to compare immune activation and infiltration in both secondary lymphoid tissue and in tumors, staining for markers including but not limited to CD4, CD8, CD25, CD69, CD44, CD62L, TCF1, TOX, TIM3, PD1. The abundance and activation state of immune cells are quantified using flow cytometry.

pSTAT5 Flow Cytometry Assay in Mouse Whole Blood

Whole blood is drawn into EDTA powder coated tubes by cardiac puncture from mice on day 7 of dosing with indicated compound. 100 μL of whole blood are stimulated with 100 ng/ml murine IL-2 for 20 minutes at 37° C., 5% CO₂. After stimulation, 1.8 mL of prewarmed BD Phosflow Lyse/Fix Buffer is added for 20 minutes at 37° C. Cells are washed twice in FACS buffer (Dulbecco's PBS with 0.2% BSA) and incubated for 30 minutes on ice in cold Perm Buffer III. Cells are washed with FACS buffer and resuspended in 50 μL of FACS buffer with antibodies and stained for 3 hours at room temperature with gentle shaking. The antibodies added are a combination of the following: anti-CD3-AF647, clone 145-2C11; anti-CD4-FITC, clone GK1.5; anti-pSTAT5 (pY694)-PE, clone 47; anti-CD45-BUV395, clone 30-F11. After staining, cells are washed twice with FACS buffer, and the samples are acquired on a BD LSRFortessa™ X20 flow cytometers (BD Biosciences, San Jose, CA) and analyzed with FLowJo V10 software (FlowJo, Ashland, OR). The mean fluorescence intensity (MFI) of pSTAT5 as a measure of the amount of phosphorylated STAT5 in the CD3+ T cell population is reported as fold-change of compound treated over vehicle treated animal groups.

Granzyme B staining of CD8 T cells Flow Cytometry Assay in Mouse Spleen

Mice are sacrificed on day 7 of dosing with compound and spleens are excised. Spleens are dissociated, red blood cells lysed, and single cell suspensions are prepared. Splenocytes are stained with Zombie UV™ Fixable Viability kit diluted in Dulbecco's PBS for 10 minutes at room temperature to exclude dead cells followed by staining for surface markers for 45 minutes on ice using the following flow cytometry antibodies diluted in autoMACS® Running Buffer (Miltenyi Biotec, Bergisch Gladbach, Germany): Brilliant Violet 510-labeled anti-CD45, Brilliant Ultraviolet 395-labeled anti-CD3, Brilliant Violet 786-labeled anti-CD4, APC/Cy7-labeled anti-CD8. Cells are washed twice with autoMACS® Running Buffer, permeabilized with Fixation/Permeabilization buffer (FoxP3/Transcription Factor Staining Buffer Set) and stained intracellularly with PE-labeled anti-Granzyme B antibody diluted in Permeabilization buffer (FoxP3/Transcription Factor Staining Buffer Set) for 1 hour on ice. After staining, cells are washed twice with autoMACS® Running Buffer, and the samples are acquired on a BD LSRFortessa™ X20 flow cytometers (BD Biosciences, San Jose, CA) and analyzed with FLowJo V10 software (FlowJo, Ashland, OR).

### Cytokine Measurement in Mouse Plasma

Whole blood is drawn into sodium heparin by cardiac puncture from mice on day 7 of dosing with compound and plasma is prepared by centrifugation. Cytokines in plasma are measured using the Th1/Th2 Cytokine & Chemokine 20-Plex Mouse ProcartaPlex™ Panel 1 (Invitrogen, Carlsbad, CA). IP10 levels are expressed as fold-changes over the vehicle control animal group.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention. It is intended that the following claims define the scope of the invention and that methods and structures within the scope of these claims and their equivalents be covered thereby.

1. A compound of Formula (I): 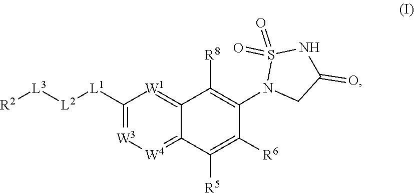
or a pharmaceutically acceptable salt or solvate thereof, wherein:
W¹ is N, W³ is N, and W⁴ is C(R⁴); W¹ is N, W³ is C(R³), and W⁴ is N; or W¹ is C(R¹), W³ is N, and W⁴ is N;
R¹, R³, R⁴, R⁵, R⁶, and R⁸ are independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —S(O)(NR¹²)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
L¹ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—;
L² is selected from C₁₋₆ alkylene, C₂₋₆ alkenylene, C₂₋₆ alkynylene, —C₀₋₃ alkylene-C3-8 carbocycle-, and —C₀₋₃ alkylene-(3- to 8-membered heterocycle)-, each of which is optionally substituted with one, two, or three R²⁰;
L³ is selected from absent, —O—, —S—, —N(R¹²)—, —C(NR¹²)—, —N(R¹²)C(NR¹²)—, —C(NR¹²)N(R¹²)—, —N(R¹²)C(NR¹²)N(R¹²)—, —C(O)O—, —OC(O)O—, —OC(O)N(R¹²)—, —N(R¹²)C(O)N(R¹²)—, —N(R¹²)C(O)O—, —C(O)N(R¹²)C(O)—, —C(O)N(R¹²)C(O)N(R¹²)—, —N(R¹²)S(O)₂—, —C(O)—, —S(O)—, —OC(O)—, —C(O)N(R¹²)—, —C(O)C(O)N(R¹²)—, —N(R¹²)C(O)—, —S(O)₂—, —OS(O)—, —S(O)O—, —OS(O)₂—, —S(O)₂O—, —S(O)(NR¹²)—, —S(O)₂N(R¹²)—, —S(O)(NR¹²)N(R¹²)—, —N(R¹²)S(O)—, —S(O)N(R¹²)—, —N(R¹²)S(O)₂N(R¹²)—, —N(R¹²)S(O)N(R¹²)—, —P(O)(OR¹²)—, and —P(O)(R¹²)—;
R² is selected from hydrogen, halogen, —CN, C₁₋₆ alkyl, C₂₋₆ alkenyl, C₂₋₆ alkynyl, C₃₋₁₀ carbocycle, and 3to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, —C₀₋₃ alkyl-C₃₋₁₀ carbocycle, —C₀₋₃ alkyl-(3- to 10-membered heterocycle), —OR²², —SR²², —N(R²²)(R²³), ═NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³)—, —S(═O)(═NR²²)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, —C₀₋₃ alkyl-C₃₋₁₀ carbocycle, and —C₀₋₃ alkyl-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)(NR²²)R²⁵, —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, C₃₋₁₀ carbocycle, and 3- to 10-membered heterocycle, or two R²¹ are taken together with the carbon atom to which they are attached to form C3-8 carbocycle or 3- to 8-membered heterocycle, each of which is optionally substituted with one, two, or three substituents independently selected from halogen, C₁₋₃ alkyl, C₁₋₃ haloalkyl, and —OH;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

2. (canceled)

3. The compound of claim 1, or a pharmaceutically acceptable salt or solvate thereof, wherein R¹, R³, and R⁴ are independently selected from hydrogen, halogen, C₁₋₃ alkyl, C₁₋₃ haloalkyl, C₃₋₆ carbocycle, —OH, —OCH₃, —NH₂, and —NHCH₃.

4-8. (canceled)

9. The compound of claim 1, wherein the compound of Formula (I) is a compound of Formula (I-A): 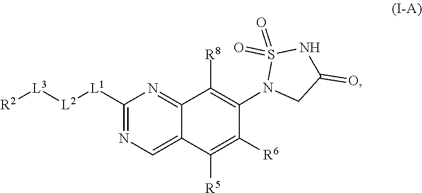
or a pharmaceutically acceptable salt or solvate thereof.

10. The compound of claim 1, wherein the compound of Formula (I) is a compound of Formula (I-B): 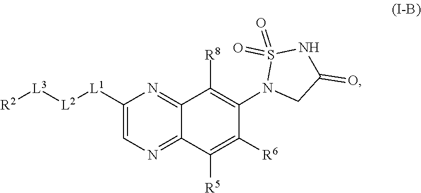
or a pharmaceutically acceptable salt or solvate thereof.

11. The compound of claim 1, wherein the compound of Formula (I) is a compound of Formula (I-C): 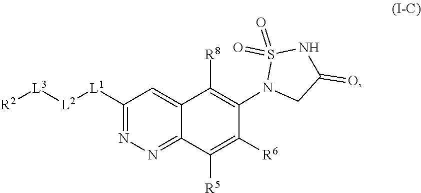
or a pharmaceutically acceptable salt or solvate thereof.

12. (canceled)

13. The compound of claim 1, or a pharmaceutically acceptable salt or solvate thereof, wherein R⁵ is selected from hydrogen, halogen, and —OH.

14. (canceled)

15. The compound of claim 1, or a pharmaceutically acceptable salt or solvate thereof, wherein R⁶ is selected from halogen, —OR¹², and C₁₋₆ alkyl, wherein C₁₋₆ alkyl is optionally substituted with one, two, or three R²⁰.

16. (canceled)

17. The compound of claim 1, or a pharmaceutically acceptable salt or solvate thereof, wherein R⁸ is selected from halogen, —OR¹², and C₁₋₆ alkyl, wherein C₁₋₆ alkyl is optionally substituted with one, two, or three R²⁰.

18. (canceled)

19. (canceled)

20. The compound of claim 1, or a pharmaceutically acceptable salt or solvate thereof, wherein R⁵ is hydrogen, R⁶ is —OH, and R⁸ is fluorine.

21. The compound of claim 1, or a pharmaceutically acceptable salt or solvate thereof, wherein R⁵ is —OH, R⁶ is hydrogen, and R⁸ is fluorine.

22. (canceled)

23. The compound of claim 1, or a pharmaceutically acceptable salt or solvate thereof, wherein L¹ is selected from absent, —O—, and —N(R¹²)—.

24-27. (canceled)

28. The compound of claim 1, or a pharmaceutically acceptable salt or solvate thereof, wherein L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰.

29-31. (canceled)

32. The compound of claim 1, or a pharmaceutically acceptable salt or solvate thereof, wherein L³ is selected from absent, —N(R¹²)—, —C(O)O—, —OC(O)—, and —S(O)₂—.

33. (canceled)

34. The compound of claim 1, or a pharmaceutically acceptable salt or solvate thereof, wherein:
L¹ is selected from absent, —O—, and —N(R¹²)—;
L² is selected from C₁₋₆ alkylene, —C₀₋₃ alkylene-C₃₋₈ carbocycle-, and —C₀₋₃ alkylene-(3- to 8-membered heterocycle)-, each of which is optionally substituted with one, two, or three R²⁰; and
L³ is selected from absent, —N(R¹²)—, —C(O)O—, —OC(O)—, and —S(O)₂—.

35. (canceled)

36. (canceled)

37. The compound of claim 1, or a pharmaceutically acceptable salt or solvate thereof, wherein R² is selected from hydrogen, halogen, —CN, C₁₋₆ alkyl, C₃₋₈ carbocycle, and 3- to 8-membered heterocycle, wherein C₁₋₆ alkyl, C₃₋₈ carbocycle, and 3- to 8-membered heterocycle are optionally substituted with one, two, or three R²⁰.

38-44. (canceled)

45. A compound of the formula 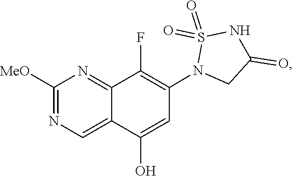 [SMILES: COc1ncc2c(O)cc(N3CC(=O)NS3(=O)=O)c(F)c2n1]
or a pharmaceutically acceptable salt or solvate thereof.

46. A compound of the formula 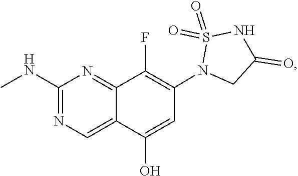 [SMILES: CNc1ncc2c(O)cc(N3CC(=O)NS3(=O)=O)c(F)c2n1]
or a pharmaceutically acceptable salt or solvate thereof.

47. The compound of claim 1, wherein the compound is selected from 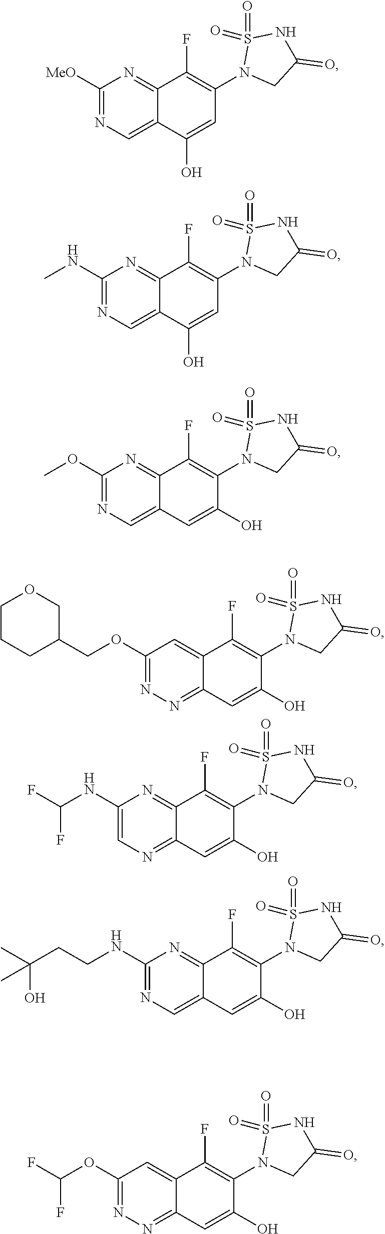 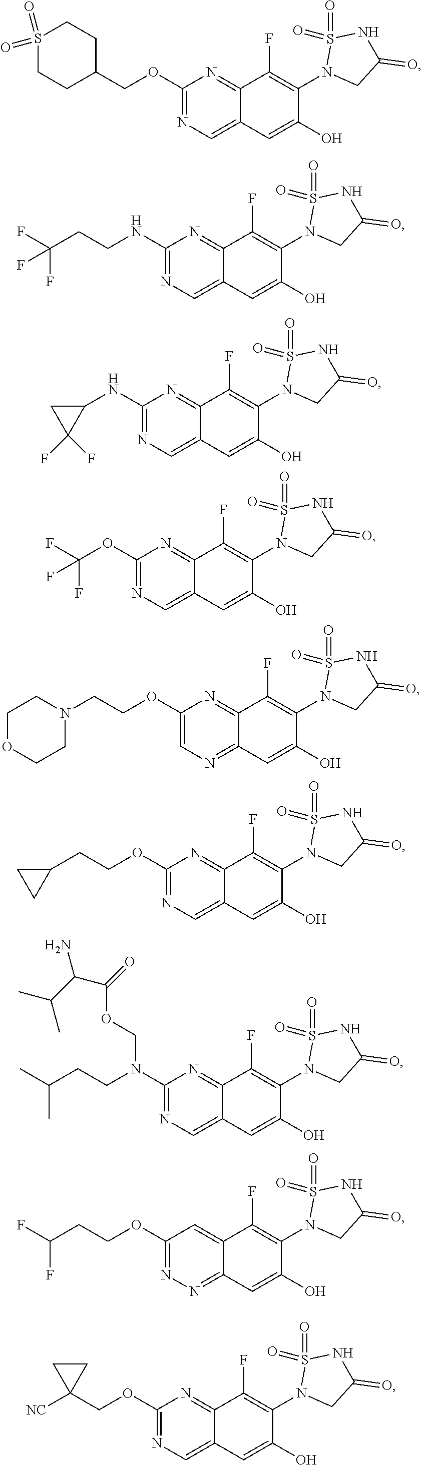 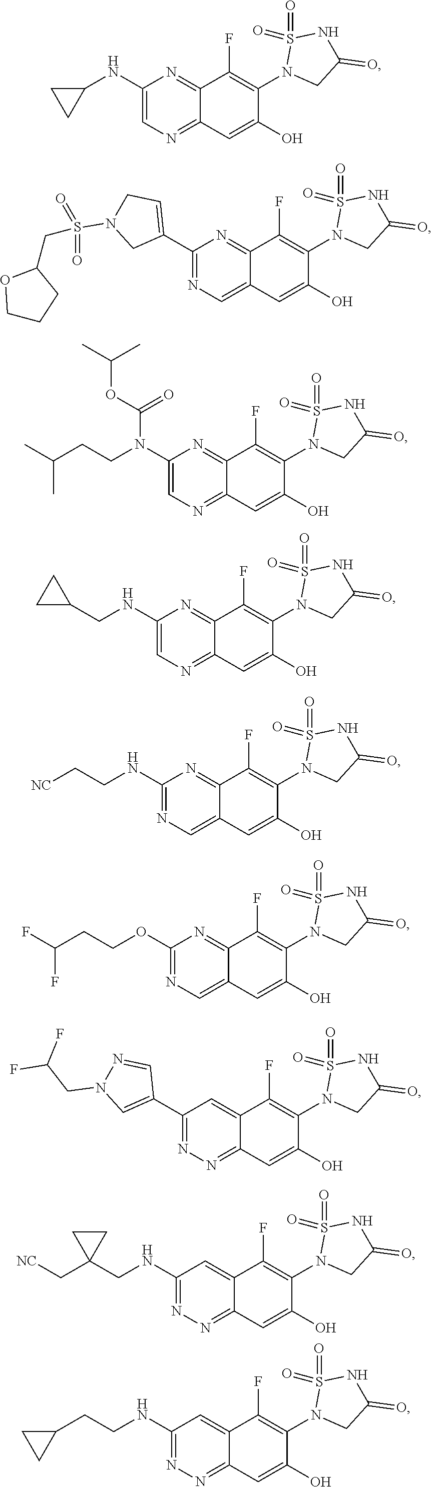 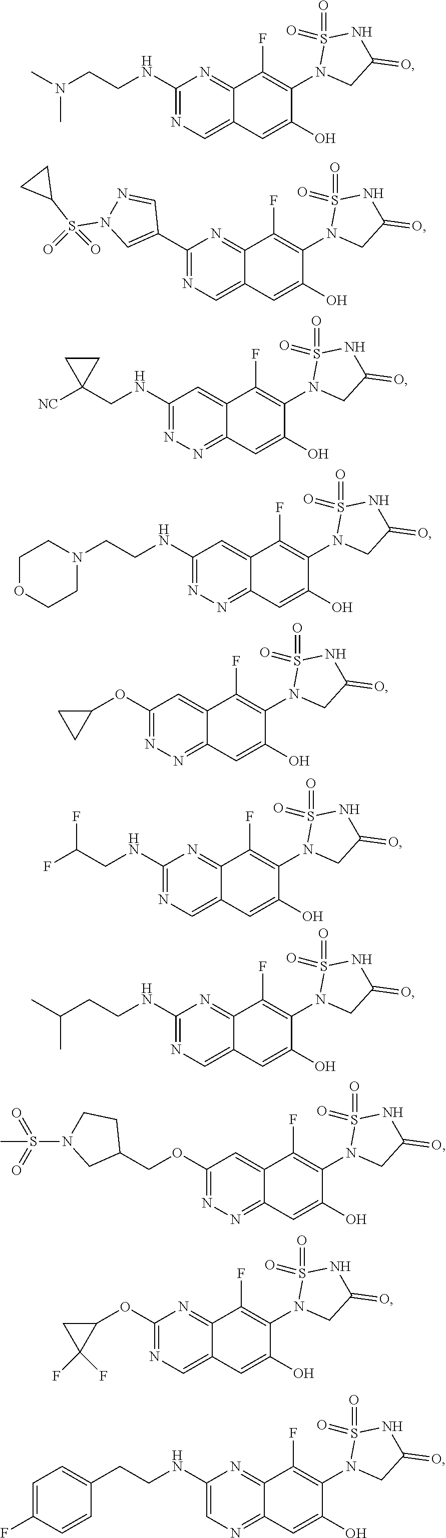 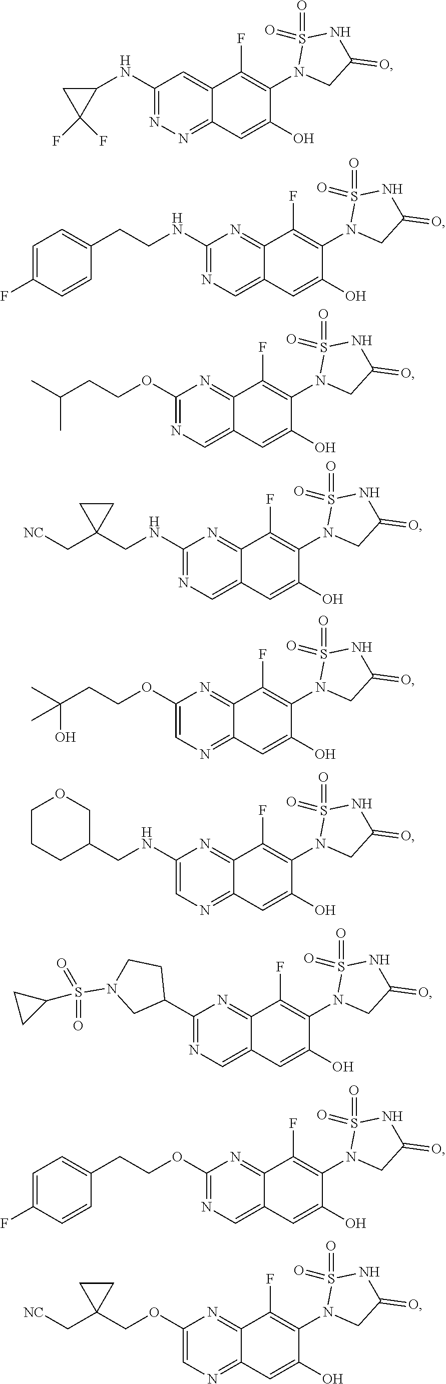 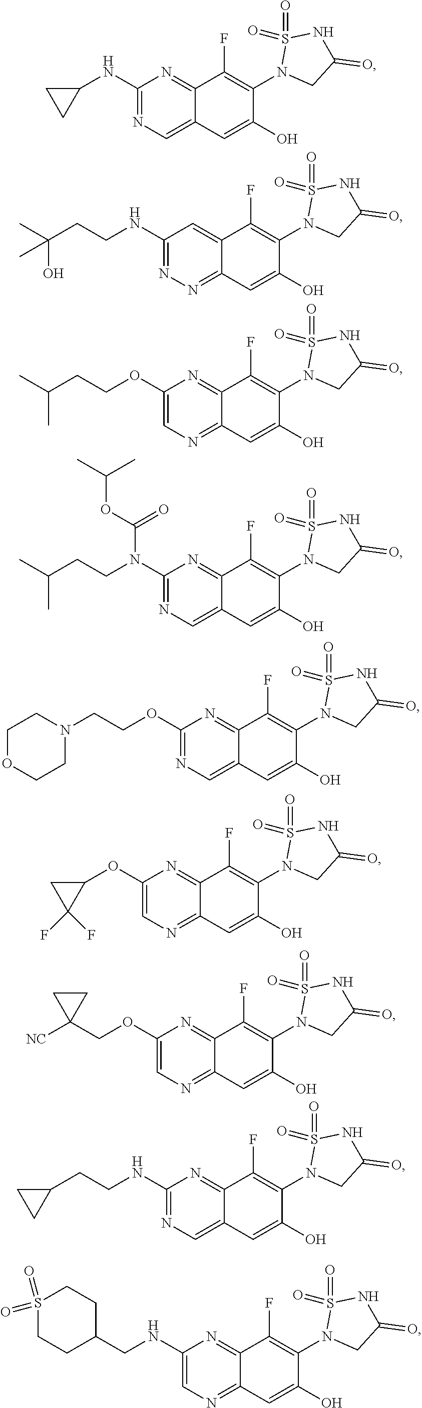 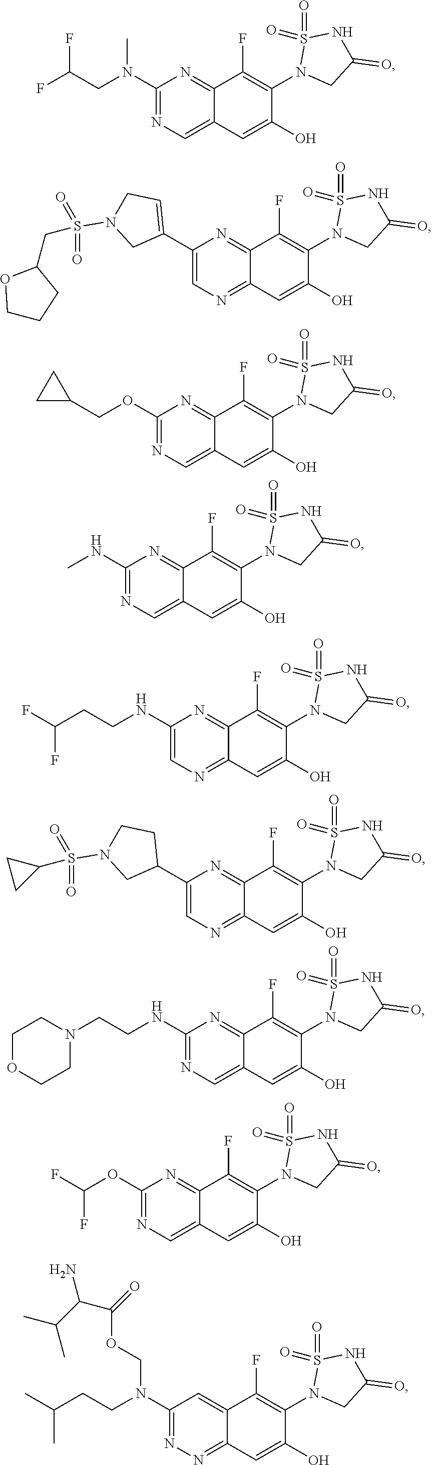 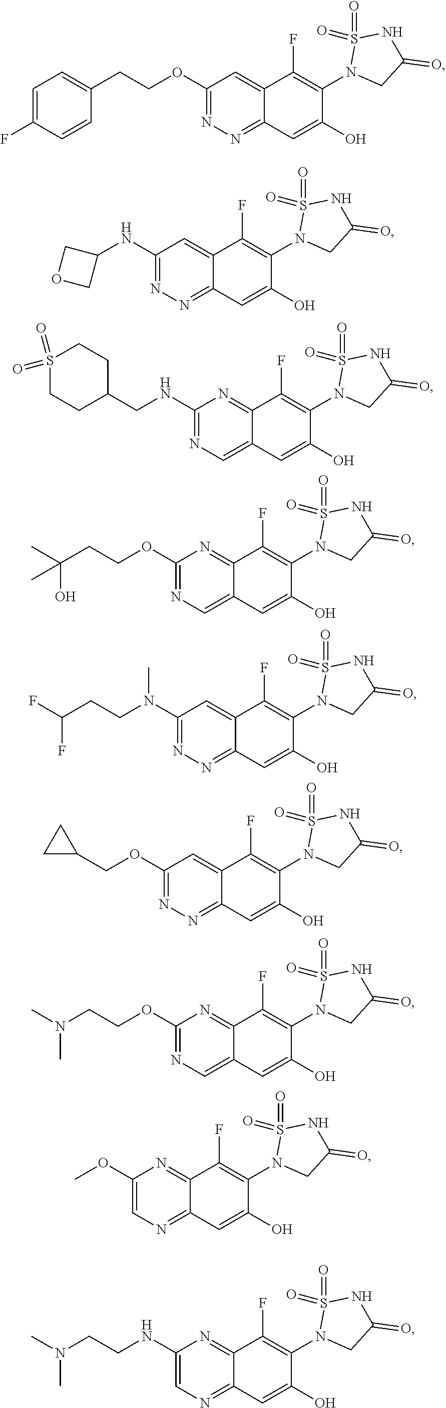 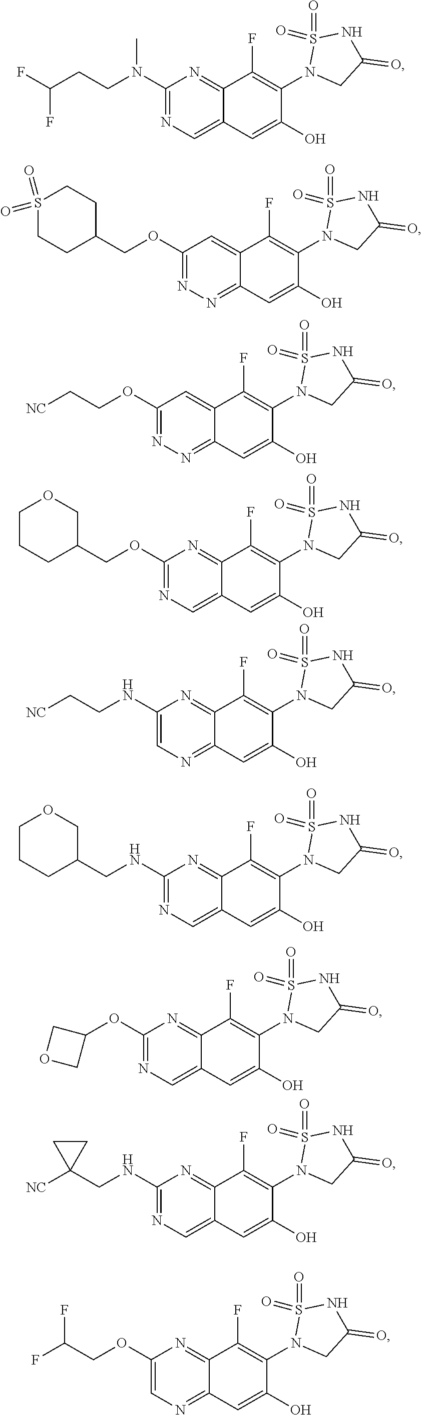 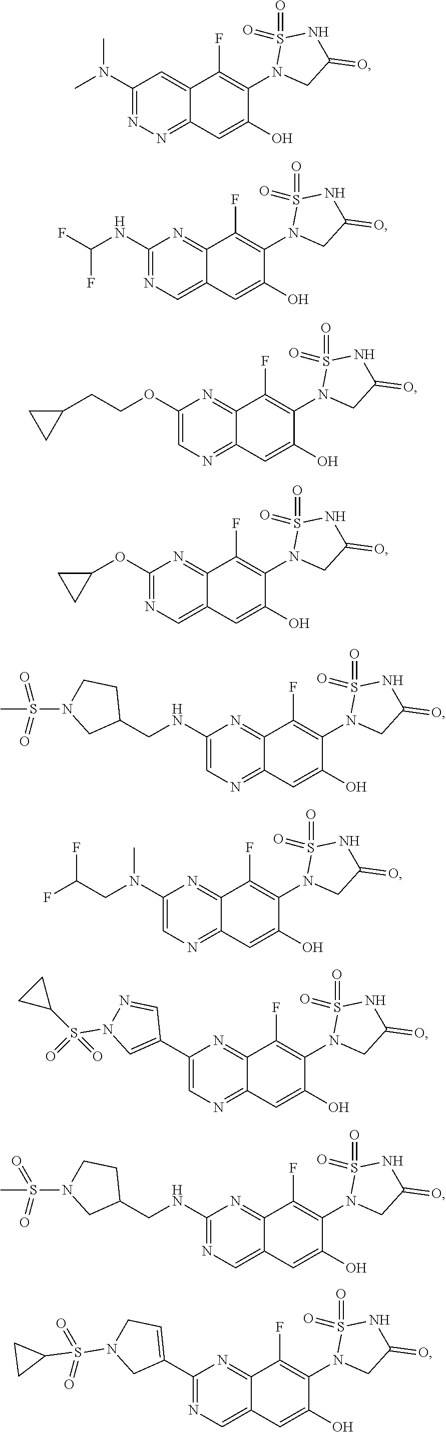 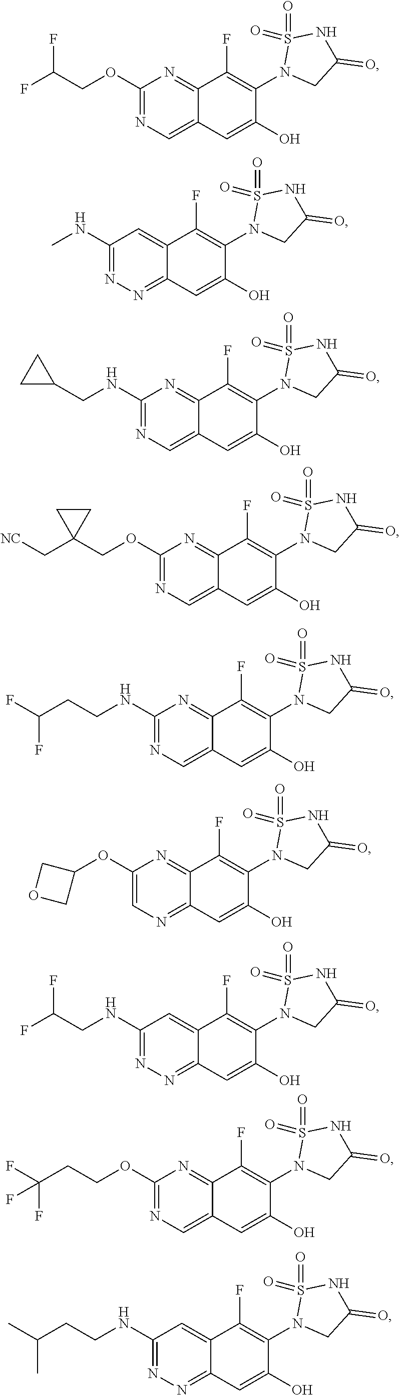 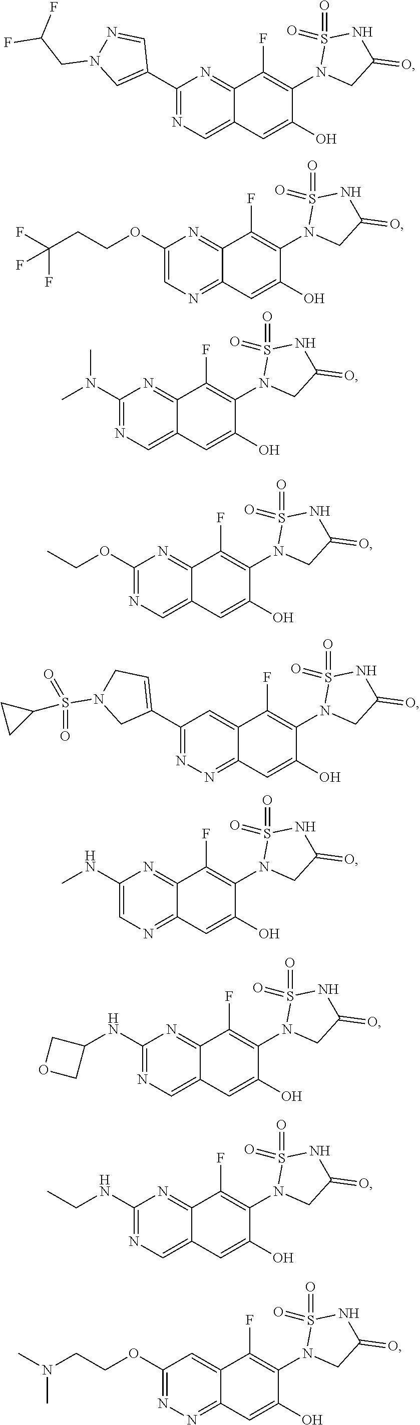 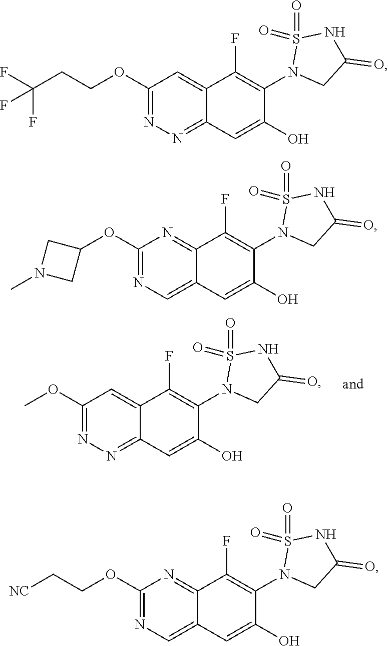
or a pharmaceutically acceptable salt or solvate thereof.

48. A pharmaceutical composition comprising a compound of claim 1, or a pharmaceutically acceptable salt or solvate thereof, and a pharmaceutically acceptable excipient.

49-76. (canceled)

77. A method of potentiating immunity of a subject in need thereof, comprising downregulating expression or activity of PTPN2 and/or PTPIB in a cell of the subject with a compound of claim 1, thereby potentiating immunity of the subject.
